SEQUENCE LISTING (129 sequences)

SEQ ID NO: 1 - protein, 401 aa, Artificial Sequence
  Synthetic Construct
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTLQRDFTAAFPRMLTTRGHGPAETQTLGSLPDINLTQISTLANELRDSRLANDLRDSGATIRIGPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 2 - protein, 492 aa, Artificial Sequence
  Synthetic Construct
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTLQRDFTAAFPRMLTTRGHGPAETQTLGSLPDINLTQISTLANELRDSRLANDLRDSGATIRIGIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 3 - protein, 487 aa, Artificial Sequence
  Synthetic Construct
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTLQRDFTAAFPRMLTTRGHGPAETQTLGSLPDINLTQISTLANELRDSRLANDLRDSGATIRIGGWNPHLLLLLLLVIVFIPAFWSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 4 - protein, 421 aa, Artificial Sequence
  Synthetic Construct
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTLQRDFTAAFPRMLTTRGHGPAETQTLGSLPDINLTQISTLANELRDSRLANDLRDSGATIRIGWMAFVAPSICIAIIMVGIFSTHYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 5 - protein, 480 aa, Artificial Sequence
  Synthetic Construct
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTLQRDFTAAFPRMLTTRGHGPAETQTLGSLPDINLTQISTLANELRDSRLANDLRDSGATIRIGAIVVPVCLAFLLTTLLGVLFCFNKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 6 - protein, 250 aa, Artificial Sequence
  Synthetic Construct
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTLQRDFTAAFPRMLTTRGHGPAETQTLGSLPDINLTQISTLANELRDSRLANDLRDSGATIRIGILVIFSGMFLVFTLAGALFLHQRRKYRSNKGESPVEPAEPCRYSCPREEEGSTIPIQEDYRKPEPACSP

SEQ ID NO: 7 - protein, 240 aa, Artificial Sequence
  Synthetic Construct
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTLQRDFTAAFPRMLTTRGHGPAETQTLGSLPDINLTQISTLANELRDSRLANDLRDSGATIRIGFWLPIGCAAFVVVCILGCILICWLTKKKYSSSVHDPNGEYMFMRAVNTAKKSRLTDVTL

SEQ ID NO: 8 - protein, 244 aa, Artificial Sequence
  Synthetic Construct
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTLQRDFTAAFPRMLTTRGHGPAETQTLGSLPDINLTQISTLANELRDSRLANDLRDSGATIRIGVAAILGLGLVLGLLGPLAILLALYLLRRDQRLPPDAHKPPGGGSFRTPIQEEQADAHSTLAKI

SEQ ID NO: 9 - protein, 648 aa, Artificial Sequence
  Synthetic Construct
LQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 10 - protein, 739 aa, Artificial Sequence
  Synthetic Construct
LQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 11 - protein, 734 aa, Artificial Sequence
  Synthetic Construct
LQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLGWNPHLLLLLLLVIVFIPAFWSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 12 - protein, 668 aa, Artificial Sequence
  Synthetic Construct
LQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLWMAFVAPSICIAIIMVGIFSTHYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 13 - protein, 727 aa, Artificial Sequence
  Synthetic Construct
LQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLAIVVPVCLAFLLTTLLGVLFCFNKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 14 - protein, 497 aa, Artificial Sequence
  Synthetic Construct
LQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLILVIFSGMFLVFTLAGALFLHQRRKYRSNKGESPVEPAEPCRYSCPREEEGSTIPIQEDYRKPEPACSP

SEQ ID NO: 15 - protein, 487 aa, Artificial Sequence
  Synthetic Construct
LQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLFWLPIGCAAFVVVCILGCILICWLTKKKYSSSVHDPNGEYMFMRAVNTAKKSRLTDVTL

SEQ ID NO: 16 - protein, 491 aa, Artificial Sequence
  Synthetic Construct
LQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLVAAILGLGLVLGLLGPLAILLALYLLRRDQRLPPDAHKPPGGGSFRTPIQEEQADAHSTLAKI

SEQ ID NO: 17 - protein, 352 aa, Artificial Sequence
  Synthetic Construct
TPEVWVQVRMEATELSSFTIRCGFLGSGSISLVTVSWGGPNGAGGTTLAVLHPERGIRQWAPARQARWETQSSISLILEGSGASSPCANTTFCCKFASFPEGSWEACGSLPPSSDPGLSAPPTPAPILRADLPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 18 - protein, 443 aa, Artificial Sequence
  Synthetic Construct
TPEVWVQVRMEATELSSFTIRCGFLGSGSISLVTVSWGGPNGAGGTTLAVLHPERGIRQWAPARQARWETQSSISLILEGSGASSPCANTTFCCKFASFPEGSWEACGSLPPSSDPGLSAPPTPAPILRADLIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 19 - protein, 438 aa, Artificial Sequence
  Synthetic Construct
TPEVWVQVRMEATELSSFTIRCGFLGSGSISLVTVSWGGPNGAGGTTLAVLHPERGIRQWAPARQARWETQSSISLILEGSGASSPCANTTFCCKFASFPEGSWEACGSLPPSSDPGLSAPPTPAPILRADLGWNPHLLLLLLLVIVFIPAFWSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 20 - protein, 372 aa, Artificial Sequence
  Synthetic Construct
TPEVWVQVRMEATELSSFTIRCGFLGSGSISLVTVSWGGPNGAGGTTLAVLHPERGIRQWAPARQARWETQSSISLILEGSGASSPCANTTFCCKFASFPEGSWEACGSLPPSSDPGLSAPPTPAPILRADLWMAFVAPSICIAIIMVGIFSTHYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 21 - protein, 431 aa, Artificial Sequence
  Synthetic Construct
TPEVWVQVRMEATELSSFTIRCGFLGSGSISLVTVSWGGPNGAGGTTLAVLHPERGIRQWAPARQARWETQSSISLILEGSGASSPCANTTFCCKFASFPEGSWEACGSLPPSSDPGLSAPPTPAPILRADLAIVVPVCLAFLLTTLLGVLFCFNKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 22 - protein, 201 aa, Artificial Sequence
  Synthetic Construct
TPEVWVQVRMEATELSSFTIRCGFLGSGSISLVTVSWGGPNGAGGTTLAVLHPERGIRQWAPARQARWETQSSISLILEGSGASSPCANTTFCCKFASFPEGSWEACGSLPPSSDPGLSAPPTPAPILRADLILVIFSGMFLVFTLAGALFLHQRRKYRSNKGESPVEPAEPCRYSCPREEEGSTIPIQEDYRKPEPACSP

SEQ ID NO: 23 - protein, 191 aa, Artificial Sequence
  Synthetic Construct
TPEVWVQVRMEATELSSFTIRCGFLGSGSISLVTVSWGGPNGAGGTTLAVLHPERGIRQWAPARQARWETQSSISLILEGSGASSPCANTTFCCKFASFPEGSWEACGSLPPSSDPGLSAPPTPAPILRADLFWLPIGCAAFVVVCILGCILICWLTKKKYSSSVHDPNGEYMFMRAVNTAKKSRLTDVTL

SEQ ID NO: 24 - protein, 195 aa, Artificial Sequence
  Synthetic Construct
TPEVWVQVRMEATELSSFTIRCGFLGSGSISLVTVSWGGPNGAGGTTLAVLHPERGIRQWAPARQARWETQSSISLILEGSGASSPCANTTFCCKFASFPEGSWEACGSLPPSSDPGLSAPPTPAPILRADLVAAILGLGLVLGLLGPLAILLALYLLRRDQRLPPDAHKPPGGGSFRTPIQEEQADAHSTLAKI

SEQ ID NO: 25 - protein, 375 aa, Artificial Sequence
  Synthetic Construct
ITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASPCSPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 26 - protein, 466 aa, Artificial Sequence
  Synthetic Construct
ITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASPCSIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 27 - protein, 461 aa, Artificial Sequence
  Synthetic Construct
ITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASPCSGWNPHLLLLLLLVIVFIPAFWSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 28 - protein, 395 aa, Artificial Sequence
  Synthetic Construct
ITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASPCSWMAFVAPSICIAIIMVGIFSTHYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 29 - protein, 454 aa, Artificial Sequence
  Synthetic Construct
ITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASPCSAIVVPVCLAFLLTTLLGVLFCFNKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 30 - protein, 224 aa, Artificial Sequence
  Synthetic Construct
ITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASPCSILVIFSGMFLVFTLAGALFLHQRRKYRSNKGESPVEPAEPCRYSCPREEEGSTIPIQEDYRKPEPACSP

SEQ ID NO: 31 - protein, 214 aa, Artificial Sequence
  Synthetic Construct
ITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASPCSFWLPIGCAAFVVVCILGCILICWLTKKKYSSSVHDPNGEYMFMRAVNTAKKSRLTDVTL

SEQ ID NO: 32 - protein, 218 aa, Artificial Sequence
  Synthetic Construct
ITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASPCSVAAILGLGLVLGLLGPLAILLALYLLRRDQRLPPDAHKPPGGGSFRTPIQEEQADAHSTLAKI

SEQ ID NO: 33 - DNA, 1206 bp, Artificial Sequence
  Synthetic Construct
tcagaagtggaatacagagcggaggtcggtcagaatgcctatctgccctgcttctacaccccagccgccccagggaacctcgtgcccgtctgctggggcaaaggagcctgtcctgtgtttgaatgtggcaacgtggtgctcaggactgatgaaagggatgtgaattattggacatccagatactggctaaatggggatttccgcaaaggagatgtgtccctgaccatagagaatgtgactctagcagacagtgggatctactgctgccggatccaaatcccaggcataatgaatgatgaaaaatttaacctgaagttggtcatcaaaccagccaaggtcacccctgcaccgactctgcagagagacttcactgcagcctttccaaggatgcttaccaccaggggacatggcccagcagagacacagacactggggagcctccctgatataaatctaacacaaatatccacattggccaatgagttacgggactctagattggccaatgacttacgggactctggagcaaccatcagaataggccctatcttactaaccatcagcattttgagttttttctctgtcgctctgttggtcatcttggcctgtgtgttatggaaaaaaaggattaagcctatcgtatggcccagtctccccgatcataagaagactctggaacatctttgtaagaaaccaagaaaaaatttaaatgtgagtttcaatcctgaaagtttcctggactgccagattcatagggtggatgacattcaagctagagatgaagtggaaggttttctgcaagatacgtttcctcagcaactagaagaatctgagaagcagaggcttggaggggatgtgcagagccccaactgcccatctgaggatgtagtcgtcactccagaaagctttggaagagattcatccctcacatgcctggctgggaatgtcagtgcatgtgacgcccctattctctcctcttccaggtccctagactgcagggagagtggcaagaatgggcctcatgtgtaccaggacctcctgcttagccttgggactacaaacagcacgctgccccctccattttctctccaatctggaatcctgacattgaacccagttgctcagggtcagcccattcttacttccctgggatcaaatcaagaagaagcatatgtcaccatgtccagcttctaccaaaaccagtga

SEQ ID NO: 34 - DNA, 1479 bp, Artificial Sequence
  Synthetic Construct
tcagaagtggaatacagagcggaggtcggtcagaatgcctatctgccctgcttctacaccccagccgccccagggaacctcgtgcccgtctgctggggcaaaggagcctgtcctgtgtttgaatgtggcaacgtggtgctcaggactgatgaaagggatgtgaattattggacatccagatactggctaaatggggatttccgcaaaggagatgtgtccctgaccatagagaatgtgactctagcagacagtgggatctactgctgccggatccaaatcccaggcataatgaatgatgaaaaatttaacctgaagttggtcatcaaaccagccaaggtcacccctgcaccgactctgcagagagacttcactgcagcctttccaaggatgcttaccaccaggggacatggcccagcagagacacagacactggggagcctccctgatataaatctaacacaaatatccacattggccaatgagttacgggactctagattggccaatgacttacgggactctggagcaaccatcagaataggcattccgtggctcggccacctcctcgtgggcctcagcggggcttttggcttcatcatcttagtgtacttgctgatcaactgcaggaacaccgggccatggctgaagaaggtcctgaagtgtaacaccccagacccctcgaagttcttttcccagctgagctcagagcatggaggagacgtccagaagtggctctcttcgcccttcccctcatcgtccttcagccctggcggcctggcacctgagatctcgccactagaagtgctggagagggacaaggtgacgcagctgctcctgcagcaggacaaggtgcctgagcccgcatccttaagcagcaaccactcgctgaccagctgcttcaccaaccagggttacttcttcttccacctcccggatgccttggagatagaggcctgccaggtgtactttacttacgacccctactcagaggaagaccctgatgagggtgtggccggggcacccacagggtcttccccccaacccctgcagcctctgtcaggggaggacgacgcctactgcaccttcccctccagggatgacctgctgctcttctcccccagtctcctcggtggccccagccccccaagcactgcccctgggggcagtggggccggtgaagagaggatgcccccttctttgcaagaaagagtccccagagactgggacccccagcccctggggcctcccaccccaggagtcccagacctggtggattttcagccaccccctgagctggtgctgcgagaggctggggaggaggtccctgacgctggccccagggagggagtcagtttcccctggtccaggcctcctgggcagggggagttcagggcccttaatgctcgcctgcccctgaacactgatgcctacttgtccctccaagaactccagggtcaggacccaactcacttggtgtag

SEQ ID NO: 35 - DNA, 1467 bp, Artificial Sequence
  Synthetic Construct
tcagaagtggaatacagagcggaggtcggtcagaatgcctatctgccctgcttctacaccccagccgccccagggaacctcgtgcccgtctgctggggcaaaggagcctgtcctgtgtttgaatgtggcaacgtggtgctcaggactgatgaaagggatgtgaattattggacatccagatactggctaaatggggatttccgcaaaggagatgtgtccctgaccatagagaatgtgactctagcagacagtgggatctactgctgccggatccaaatcccaggcataatgaatgatgaaaaatttaacctgaagttggtcatcaaaccagccaaggtcacccctgcaccgactctgcagagagacttcactgcagcctttccaaggatgcttaccaccaggggacatggcccagcagagacacagacactggggagcctccctgatataaatctaacacaaatatccacattggccaatgagttacgggactctagattggccaatgacttacgggactctggagcaaccatcagaataggcggctggaaccctcacctgctgcttctcctcctgcttgtcatagtcttcattcctgccttctggagcctgaagacccatccattgtggaggctatggaagaagatatgggccgtccccagccctgagcggttcttcatgcccctgtacaagggctgcagcggagacttcaagaaatgggtgggtgcacccttcactggctccagcctggagctgggaccctggagcccagaggtgccctccaccctggaggtgtacagctgccacccaccacggagcccggccaagaggctgcagctcacggagctacaagaaccagcagagctggtggagtctgacggtgtgcccaagcccagcttctggccgacagcccagaactcggggggctcagcttacagtgaggagagggatcggccatacggcctggtgtccattgacacagtgactgtgctagatgcagaggggccatgcacctggccctgcagctgtgaggatgacggctacccagccctggacctggatgctggcctggagcccagcccaggcctagaggacccactcttggatgcagggaccacagtcctgtcctgtggctgtgtctcagctggcagccctgggctaggagggcccctgggaagcctcctggacagactaaagccaccccttgcagatggggaggactgggctgggggactgccctggggtggccggtcacctggaggggtctcagagagtgaggcgggctcacccctggccggcctggatatggacacgtttgacagtggctttgtgggctctgactgcagcagccctgtggagtgtgacttcaccagccccggggacgaaggacccccccggagctacctccgccagtgggtggtcattcctccgccactttcgagccctggaccccaggccagctaatga

SEQ ID NO: 36 - DNA, 1266 bp, Artificial Sequence
  Synthetic Construct
tcagaagtggaatacagagcggaggtcggtcagaatgcctatctgccctgcttctacaccccagccgccccagggaacctcgtgcccgtctgctggggcaaaggagcctgtcctgtgtttgaatgtggcaacgtggtgctcaggactgatgaaagggatgtgaattattggacatccagatactggctaaatggggatttccgcaaaggagatgtgtccctgaccatagagaatgtgactctagcagacagtgggatctactgctgccggatccaaatcccaggcataatgaatgatgaaaaatttaacctgaagttggtcatcaaaccagccaaggtcacccctgcaccgactctgcagagagacttcactgcagcctttccaaggatgcttaccaccaggggacatggcccagcagagacacagacactggggagcctccctgatataaatctaacacaaatatccacattggccaatgagttacgggactctagattggccaatgacttacgggactctggagcaaccatcagaataggctggatggcgtttgtggcaccaagcatttgcattgctatcatcatggtgggcattttctcaacgcattacttccagcaaaaggtgtttgttctcctagcagccctcagacctcagtggtgtagcagagaaattccagatccagcaaatagcacttgcgctaagaaatatcccattgcagaggagaagacacagctgcccttggacaggctcctgatagactggcccacgcctgaagatcctgaaccgctggtcatcagtgaagtccttcatcaagtgaccccagttttcagacatcccccctgctccaactggccacaaagggaaaaaggaatccaaggtcatcaggcctctgagaaagacatgatgcacagtgcctcaagcccaccacctccaagagctctccaagctgagagcagacaactggtggatctgtacaaggtgctggagagcaggggctccgacccaaagccagaaaacccagcctgtccctggacggtgctcccagcaggtgaccttcccacccatgatggctacttaccctccaacatagatgacctcccctcacatgaggcacctctcgctgactctctggaagaactggagcctcagcacatctccctttctgttttcccctcaagttctcttcacccactcaccttctcctgtggtgataagctgactctggatcagttaaagatgaggtgtgactccctcatgctctga

SEQ ID NO: 37 - DNA, 1441 bp, Artificial Sequence
  Synthetic Construct
tcagaagtggaatacagagcggaggtcggtcagaatgcctatctgccctgcttctacaccccagccgccccagggaacctcgtgcccgtctgctggggcaaaggagcctgtcctgtgtttgaatgtggcaacgtggtgctcaggactgatgaaagggatgtgaattattggacatccagatactggctaaatggggatttccgcaaaggagatgtgtccctgaccatagagaatgtgactctagcagacagtgggatctactgctgccggatccaaatcccaggcataatgaatgatgaaaaatttaacctgaagttggtcatcaaaccagccaaggtcacccctgcaccgactctgcagagagacttcactgcagcctttccaaggatgcttaccaccaggggacatggcccagcagagacacagacactggggagcctccctgatataaatctaacacaaatatccacattggccaatgagttacgggactctagattggccaatgacttacgggactctggagcaaccatcagaataggcgccatagtcgtgcctgtttgcttagcattcctattgacaactcttctgggagtgctgttctgctttaataagcgagacctaattaaaaaacacatctggcctaatgttccagatccttcaaagagtcatattgcccagtggtcacctcacactcctccaaggcacaattttaattcaaaagatcaaatgtattcagatggcaatttcactgatgtaagtgttgtggaaatagaagcaaatgacaaaaagccttttccagaagatctgaaatcattggacctgttcaaaaaggaaaaaattaatactgaaggacacagcagtggtattggggggtcttcatgcatgtcatcttctaggccaagcatttctagcagtgatgaaaatgaatcttcacaaaacacttcgagcactgtccagtattctaccgtggtacacagtggctacagacaccaagttccgtcagtccaagtcttctcaagatccgagtctacccagcccttgttagattcagaggagcggccagaagatctacaattagtagatcatgtagatggcggtgatggtattttgcccaggcaacagtacttcaaacagaactgcagtcagcatgaatccagtccagatatttcacattttgaaaggtcaaagcaagtttcatcagtcaatgaggaagattttgttagacttaaacagcagatttcagatcatatttcacaatcctgtggatctgggcaaatgaaaatgtttcaggaagtttctgcagcagatgcttttggtccaggtactgagggacaagtagaaagatttgaaacagttggcatggaggctgcgactgatgaaggcatgcctaaaagttacttaccacagactgtacggcaaggcggctacatgcctcagt

SEQ ID NO: 38 - DNA, 753 bp, Artificial Sequence
  Synthetic Construct
tcagaagtggaatacagagcggaggtcggtcagaatgcctatctgccctgcttctacaccccagccgccccagggaacctcgtgcccgtctgctggggcaaaggagcctgtcctgtgtttgaatgtggcaacgtggtgctcaggactgatgaaagggatgtgaattattggacatccagatactggctaaatggggatttccgcaaaggagatgtgtccctgaccatagagaatgtgactctagcagacagtgggatctactgctgccggatccaaatcccaggcataatgaatgatgaaaaatttaacctgaagttggtcatcaaaccagccaaggtcacccctgcaccgactctgcagagagacttcactgcagcctttccaaggatgcttaccaccaggggacatggcccagcagagacacagacactggggagcctccctgatataaatctaacacaaatatccacattggccaatgagttacgggactctagattggccaatgacttacgggactctggagcaaccatcagaataggcatccttgtgatcttctctggaatgttccttgttttcaccctggccggggccctgttcctccatcaacgaaggaaatatagatcaaacaaaggagaaagtcctgtggagcctgcagagccttgtcgttacagctgccccagggaggaggagggcagcaccatccccatccaggaggattaccgaaaaccggagcctgcctgctccccctga

SEQ ID NO: 39 - DNA, 723 bp, Artificial Sequence
  Synthetic Construct
tcagaagtggaatacagagcggaggtcggtcagaatgcctatctgccctgcttctacaccccagccgccccagggaacctcgtgcccgtctgctggggcaaaggagcctgtcctgtgtttgaatgtggcaacgtggtgctcaggactgatgaaagggatgtgaattattggacatccagatactggctaaatggggatttccgcaaaggagatgtgtccctgaccatagagaatgtgactctagcagacagtgggatctactgctgccggatccaaatcccaggcataatgaatgatgaaaaatttaacctgaagttggtcatcaaaccagccaaggtcacccctgcaccgactctgcagagagacttcactgcagcctttccaaggatgcttaccaccaggggacatggcccagcagagacacagacactggggagcctccctgatataaatctaacacaaatatccacattggccaatgagttacgggactctagattggccaatgacttacgggactctggagcaaccatcagaataggcttctggttacccataggatgtgcagcctttgttgtagtctgcattttgggatgcatacttatttgttggcttacaaaaaagaagtattcatccagtgtgcacgaccctaacggtgaatacatgttcatgagagcagtgaacacagccaaaaaatctagactcacagatgtgaccctataa

SEQ ID NO: 40 - DNA, 735 bp, Artificial Sequence
  Synthetic Construct
tcagaagtggaatacagagcggaggtcggtcagaatgcctatctgccctgcttctacaccccagccgccccagggaacctcgtgcccgtctgctggggcaaaggagcctgtcctgtgtttgaatgtggcaacgtggtgctcaggactgatgaaagggatgtgaattattggacatccagatactggctaaatggggatttccgcaaaggagatgtgtccctgaccatagagaatgtgactctagcagacagtgggatctactgctgccggatccaaatcccaggcataatgaatgatgaaaaatttaacctgaagttggtcatcaaaccagccaaggtcacccctgcaccgactctgcagagagacttcactgcagcctttccaaggatgcttaccaccaggggacatggcccagcagagacacagacactggggagcctccctgatataaatctaacacaaatatccacattggccaatgagttacgggactctagattggccaatgacttacgggactctggagcaaccatcagaataggcgttgccgccatcctgggcctgggcctggtgctggggctgctgggccccctggccatcctgctggccctgtacctgctccggagggaccagaggctgccccccgatgcccacaagccccctgggggaggcagtttccggacccccatccaagaggagcaggccgacgcccactccaccctggccaagatctga

SEQ ID NO: 41 - DNA, 1947 bp, Artificial Sequence
  Synthetic Construct
ctccagccaggggctgaggtcccggtggtgtgggcccaggagggggctcctgcccagctcccctgcagccccacaatccccctccaggatctcagccttctgcgaagagcaggggtcacttggcagcatcagccagacagtggcccgcccgctgccgcccccggccatcccctggcccccggccctcacccggcggcgccctcctcctgggggcccaggccccgccgctacacggtgctgagcgtgggtcccggaggcctgcgcagcgggaggctgcccctgcagccccgcgtccagctggatgagcgcggccggcagcgcggggacttctcgctatggctgcgcccagcccggcgcgcggacgccggcgagtaccgcgccgcggtgcacctcagggaccgcgccctctcctgccgcctccgtctgcgcctgggccaggcctcgatgactgccagccccccaggatctctcagagcctccgactgggtcattttgaactgctccttcagccgccctgaccgcccagcctctgtgcattggttccggaaccggggccagggccgagtccctgtccgggagtccccccatcaccacttagcggaaagcttcctcttcctgccccaagtcagccccatggactctgggccctggggctgcatcctcacctacagagatggcttcaacgtctccatcatgtataacctcactgttctgggtctggagcccccaactcccttgacagtgtacgctggagcaggttccagggtggggctgccctgccgcctgcctgctggtgtggggacccggtctttcctcactgccaagtggactcctcctgggggaggccctgacctcctggtgactggagacaatggcgactttacccttcgactagaggatgtgagccaggcccaggctgggacctacacctgccatatccatctgcaggaacagcagctcaatgccactgtcacattggcaatcatcacagtgactcccaaatcctttgggtcacctggatccctggggaagctgctttgtgaggtgactccagtatctggacaagaacgctttgtgtggagctctctggacaccccatcccagaggagtttctcaggaccttggctggaggcacaggaggcccagctcctttcccagccttggcaatgccagctgtaccagggggagaggcttcttggagcagcagtgtacttcacagagctgtctagcccaggtgcccaacgctctgggagagccccaggtgccctcccagcaggccacctccctatcttactaaccatcagcattttgagttttttctctgtcgctctgttggtcatcttggcctgtgtgttatggaaaaaaaggattaagcctatcgtatggcccagtctccccgatcataagaagactctggaacatctttgtaagaaaccaagaaaaaatttaaatgtgagtttcaatcctgaaagtttcctggactgccagattcatagggtggatgacattcaagctagagatgaagtggaaggttttctgcaagatacgtttcctcagcaactagaagaatctgagaagcagaggcttggaggggatgtgcagagccccaactgcccatctgaggatgtagtcgtcactccagaaagctttggaagagattcatccctcacatgcctggctgggaatgtcagtgcatgtgacgcccctattctctcctcttccaggtccctagactgcagggagagtggcaagaatgggcctcatgtgtaccaggacctcctgcttagccttgggactacaaacagcacgctgccccctccattttctctccaatctggaatcctgacattgaacccagttgctcagggtcagcccattcttacttccctgggatcaaatcaagaagaagcatatgtcaccatgtccagcttctaccaaaaccagtga

SEQ ID NO: 42 - DNA, 2220 bp, Artificial Sequence
  Synthetic Construct
ctccagccaggggctgaggtcccggtggtgtgggcccaggagggggctcctgcccagctcccctgcagccccacaatccccctccaggatctcagccttctgcgaagagcaggggtcacttggcagcatcagccagacagtggcccgcccgctgccgcccccggccatcccctggcccccggccctcacccggcggcgccctcctcctgggggcccaggccccgccgctacacggtgctgagcgtgggtcccggaggcctgcgcagcgggaggctgcccctgcagccccgcgtccagctggatgagcgcggccggcagcgcggggacttctcgctatggctgcgcccagcccggcgcgcggacgccggcgagtaccgcgccgcggtgcacctcagggaccgcgccctctcctgccgcctccgtctgcgcctgggccaggcctcgatgactgccagccccccaggatctctcagagcctccgactgggtcattttgaactgctccttcagccgccctgaccgcccagcctctgtgcattggttccggaaccggggccagggccgagtccctgtccgggagtccccccatcaccacttagcggaaagcttcctcttcctgccccaagtcagccccatggactctgggccctggggctgcatcctcacctacagagatggcttcaacgtctccatcatgtataacctcactgttctgggtctggagcccccaactcccttgacagtgtacgctggagcaggttccagggtggggctgccctgccgcctgcctgctggtgtggggacccggtctttcctcactgccaagtggactcctcctgggggaggccctgacctcctggtgactggagacaatggcgactttacccttcgactagaggatgtgagccaggcccaggctgggacctacacctgccatatccatctgcaggaacagcagctcaatgccactgtcacattggcaatcatcacagtgactcccaaatcctttgggtcacctggatccctggggaagctgctttgtgaggtgactccagtatctggacaagaacgctttgtgtggagctctctggacaccccatcccagaggagtttctcaggaccttggctggaggcacaggaggcccagctcctttcccagccttggcaatgccagctgtaccagggggagaggcttcttggagcagcagtgtacttcacagagctgtctagcccaggtgcccaacgctctgggagagccccaggtgccctcccagcaggccacctcattccgtggctcggccacctcctcgtgggcctcagcggggcttttggcttcatcatcttagtgtacttgctgatcaactgcaggaacaccgggccatggctgaagaaggtcctgaagtgtaacaccccagacccctcgaagttcttttcccagctgagctcagagcatggaggagacgtccagaagtggctctcttcgcccttcccctcatcgtccttcagccctggcggcctggcacctgagatctcgccactagaagtgctggagagggacaaggtgacgcagctgctcctgcagcaggacaaggtgcctgagcccgcatccttaagcagcaaccactcgctgaccagctgcttcaccaaccagggttacttcttcttccacctcccggatgccttggagatagaggcctgccaggtgtactttacttacgacccctactcagaggaagaccctgatgagggtgtggccggggcacccacagggtcttccccccaacccctgcagcctctgtcaggggaggacgacgcctactgcaccttcccctccagggatgacctgctgctcttctcccccagtctcctcggtggccccagccccccaagcactgcccctgggggcagtggggccggtgaagagaggatgcccccttctttgcaagaaagagtccccagagactgggacccccagcccctggggcctcccaccccaggagtcccagacctggtggattttcagccaccccctgagctggtgctgcgagaggctggggaggaggtccctgacgctggccccagggagggagtcagtttcccctggtccaggcctcctgggcagggggagttcagggcccttaatgctcgcctgcccctgaacactgatgcctacttgtccctccaagaactccagggtcaggacccaactcacttggtgtag

SEQ ID NO: 43 - DNA, 2208 bp, Artificial Sequence
  Synthetic Construct
ctccagccaggggctgaggtcccggtggtgtgggcccaggagggggctcctgcccagctcccctgcagccccacaatccccctccaggatctcagccttctgcgaagagcaggggtcacttggcagcatcagccagacagtggcccgcccgctgccgcccccggccatcccctggcccccggccctcacccggcggcgccctcctcctgggggcccaggccccgccgctacacggtgctgagcgtgggtcccggaggcctgcgcagcgggaggctgcccctgcagccccgcgtccagctggatgagcgcggccggcagcgcggggacttctcgctatggctgcgcccagcccggcgcgcggacgccggcgagtaccgcgccgcggtgcacctcagggaccgcgccctctcctgccgcctccgtctgcgcctgggccaggcctcgatgactgccagccccccaggatctctcagagcctccgactgggtcattttgaactgctccttcagccgccctgaccgcccagcctctgtgcattggttccggaaccggggccagggccgagtccctgtccgggagtccccccatcaccacttagcggaaagcttcctcttcctgccccaagtcagccccatggactctgggccctggggctgcatcctcacctacagagatggcttcaacgtctccatcatgtataacctcactgttctgggtctggagcccccaactcccttgacagtgtacgctggagcaggttccagggtggggctgccctgccgcctgcctgctggtgtggggacccggtctttcctcactgccaagtggactcctcctgggggaggccctgacctcctggtgactggagacaatggcgactttacccttcgactagaggatgtgagccaggcccaggctgggacctacacctgccatatccatctgcaggaacagcagctcaatgccactgtcacattggcaatcatcacagtgactcccaaatcctttgggtcacctggatccctggggaagctgctttgtgaggtgactccagtatctggacaagaacgctttgtgtggagctctctggacaccccatcccagaggagtttctcaggaccttggctggaggcacaggaggcccagctcctttcccagccttggcaatgccagctgtaccagggggagaggcttcttggagcagcagtgtacttcacagagctgtctagcccaggtgcccaacgctctgggagagccccaggtgccctcccagcaggccacctcggctggaaccctcacctgctgcttctcctcctgcttgtcatagtcttcattcctgccttctggagcctgaagacccatccattgtggaggctatggaagaagatatgggccgtccccagccctgagcggttcttcatgcccctgtacaagggctgcagcggagacttcaagaaatgggtgggtgcacccttcactggctccagcctggagctgggaccctggagcccagaggtgccctccaccctggaggtgtacagctgccacccaccacggagcccggccaagaggctgcagctcacggagctacaagaaccagcagagctggtggagtctgacggtgtgcccaagcccagcttctggccgacagcccagaactcggggggctcagcttacagtgaggagagggatcggccatacggcctggtgtccattgacacagtgactgtgctagatgcagaggggccatgcacctggccctgcagctgtgaggatgacggctacccagccctggacctggatgctggcctggagcccagcccaggcctagaggacccactcttggatgcagggaccacagtcctgtcctgtggctgtgtctcagctggcagccctgggctaggagggcccctgggaagcctcctggacagactaaagccaccccttgcagatggggaggactgggctgggggactgccctggggtggccggtcacctggaggggtctcagagagtgaggcgggctcacccctggccggcctggatatggacacgtttgacagtggctttgtgggctctgactgcagcagccctgtggagtgtgacttcaccagccccggggacgaaggacccccccggagctacctccgccagtgggtggtcattcctccgccactttcgagccctggaccccaggccagctaatga

SEQ ID NO: 44 - DNA, 2007 bp, Artificial Sequence
  Synthetic Construct
ctccagccaggggctgaggtcccggtggtgtgggcccaggagggggctcctgcccagctcccctgcagccccacaatccccctccaggatctcagccttctgcgaagagcaggggtcacttggcagcatcagccagacagtggcccgcccgctgccgcccccggccatcccctggcccccggccctcacccggcggcgccctcctcctgggggcccaggccccgccgctacacggtgctgagcgtgggtcccggaggcctgcgcagcgggaggctgcccctgcagccccgcgtccagctggatgagcgcggccggcagcgcggggacttctcgctatggctgcgcccagcccggcgcgcggacgccggcgagtaccgcgccgcggtgcacctcagggaccgcgccctctcctgccgcctccgtctgcgcctgggccaggcctcgatgactgccagccccccaggatctctcagagcctccgactgggtcattttgaactgctccttcagccgccctgaccgcccagcctctgtgcattggttccggaaccggggccagggccgagtccctgtccgggagtccccccatcaccacttagcggaaagcttcctcttcctgccccaagtcagccccatggactctgggccctggggctgcatcctcacctacagagatggcttcaacgtctccatcatgtataacctcactgttctgggtctggagcccccaactcccttgacagtgtacgctggagcaggttccagggtggggctgccctgccgcctgcctgctggtgtggggacccggtctttcctcactgccaagtggactcctcctgggggaggccctgacctcctggtgactggagacaatggcgactttacccttcgactagaggatgtgagccaggcccaggctgggacctacacctgccatatccatctgcaggaacagcagctcaatgccactgtcacattggcaatcatcacagtgactcccaaatcctttgggtcacctggatccctggggaagctgctttgtgaggtgactccagtatctggacaagaacgctttgtgtggagctctctggacaccccatcccagaggagtttctcaggaccttggctggaggcacaggaggcccagctcctttcccagccttggcaatgccagctgtaccagggggagaggcttcttggagcagcagtgtacttcacagagctgtctagcccaggtgcccaacgctctgggagagccccaggtgccctcccagcaggccacctctggatggcgtttgtggcaccaagcatttgcattgctatcatcatggtgggcattttctcaacgcattacttccagcaaaaggtgtttgttctcctagcagccctcagacctcagtggtgtagcagagaaattccagatccagcaaatagcacttgcgctaagaaatatcccattgcagaggagaagacacagctgcccttggacaggctcctgatagactggcccacgcctgaagatcctgaaccgctggtcatcagtgaagtccttcatcaagtgaccccagttttcagacatcccccctgctccaactggccacaaagggaaaaaggaatccaaggtcatcaggcctctgagaaagacatgatgcacagtgcctcaagcccaccacctccaagagctctccaagctgagagcagacaactggtggatctgtacaaggtgctggagagcaggggctccgacccaaagccagaaaacccagcctgtccctggacggtgctcccagcaggtgaccttcccacccatgatggctacttaccctccaacatagatgacctcccctcacatgaggcacctctcgctgactctctggaagaactggagcctcagcacatctccctttctgttttcccctcaagttctcttcacccactcaccttctcctgtggtgataagctgactctggatcagttaaagatgaggtgtgactccctcatgctctga

SEQ ID NO: 45 - DNA, 2182 bp, Artificial Sequence
  Synthetic Construct
ctccagccaggggctgaggtcccggtggtgtgggcccaggagggggctcctgcccagctcccctgcagccccacaatccccctccaggatctcagccttctgcgaagagcaggggtcacttggcagcatcagccagacagtggcccgcccgctgccgcccccggccatcccctggcccccggccctcacccggcggcgccctcctcctgggggcccaggccccgccgctacacggtgctgagcgtgggtcccggaggcctgcgcagcgggaggctgcccctgcagccccgcgtccagctggatgagcgcggccggcagcgcggggacttctcgctatggctgcgcccagcccggcgcgcggacgccggcgagtaccgcgccgcggtgcacctcagggaccgcgccctctcctgccgcctccgtctgcgcctgggccaggcctcgatgactgccagccccccaggatctctcagagcctccgactgggtcattttgaactgctccttcagccgccctgaccgcccagcctctgtgcattggttccggaaccggggccagggccgagtccctgtccgggagtccccccatcaccacttagcggaaagcttcctcttcctgccccaagtcagccccatggactctgggccctggggctgcatcctcacctacagagatggcttcaacgtctccatcatgtataacctcactgttctgggtctggagcccccaactcccttgacagtgtacgctggagcaggttccagggtggggctgccctgccgcctgcctgctggtgtggggacccggtctttcctcactgccaagtggactcctcctgggggaggccctgacctcctggtgactggagacaatggcgactttacccttcgactagaggatgtgagccaggcccaggctgggacctacacctgccatatccatctgcaggaacagcagctcaatgccactgtcacattggcaatcatcacagtgactcccaaatcctttgggtcacctggatccctggggaagctgctttgtgaggtgactccagtatctggacaagaacgctttgtgtggagctctctggacaccccatcccagaggagtttctcaggaccttggctggaggcacaggaggcccagctcctttcccagccttggcaatgccagctgtaccagggggagaggcttcttggagcagcagtgtacttcacagagctgtctagcccaggtgcccaacgctctgggagagccccaggtgccctcccagcaggccacctcgccatagtcgtgcctgtttgcttagcattcctattgacaactcttctgggagtgctgttctgctttaataagcgagacctaattaaaaaacacatctggcctaatgttccagatccttcaaagagtcatattgcccagtggtcacctcacactcctccaaggcacaattttaattcaaaagatcaaatgtattcagatggcaatttcactgatgtaagtgttgtggaaatagaagcaaatgacaaaaagccttttccagaagatctgaaatcattggacctgttcaaaaaggaaaaaattaatactgaaggacacagcagtggtattggggggtcttcatgcatgtcatcttctaggccaagcatttctagcagtgatgaaaatgaatcttcacaaaacacttcgagcactgtccagtattctaccgtggtacacagtggctacagacaccaagttccgtcagtccaagtcttctcaagatccgagtctacccagcccttgttagattcagaggagcggccagaagatctacaattagtagatcatgtagatggcggtgatggtattttgcccaggcaacagtacttcaaacagaactgcagtcagcatgaatccagtccagatatttcacattttgaaaggtcaaagcaagtttcatcagtcaatgaggaagattttgttagacttaaacagcagatttcagatcatatttcacaatcctgtggatctgggcaaatgaaaatgtttcaggaagtttctgcagcagatgcttttggtccaggtactgagggacaagtagaaagatttgaaacagttggcatggaggctgcgactgatgaaggcatgcctaaaagttacttaccacagactgtacggcaaggcggctacatgcctcagt

SEQ ID NO: 46 - DNA, 1494 bp, Artificial Sequence
  Synthetic Construct
ctccagccaggggctgaggtcccggtggtgtgggcccaggagggggctcctgcccagctcccctgcagccccacaatccccctccaggatctcagccttctgcgaagagcaggggtcacttggcagcatcagccagacagtggcccgcccgctgccgcccccggccatcccctggcccccggccctcacccggcggcgccctcctcctgggggcccaggccccgccgctacacggtgctgagcgtgggtcccggaggcctgcgcagcgggaggctgcccctgcagccccgcgtccagctggatgagcgcggccggcagcgcggggacttctcgctatggctgcgcccagcccggcgcgcggacgccggcgagtaccgcgccgcggtgcacctcagggaccgcgccctctcctgccgcctccgtctgcgcctgggccaggcctcgatgactgccagccccccaggatctctcagagcctccgactgggtcattttgaactgctccttcagccgccctgaccgcccagcctctgtgcattggttccggaaccggggccagggccgagtccctgtccgggagtccccccatcaccacttagcggaaagcttcctcttcctgccccaagtcagccccatggactctgggccctggggctgcatcctcacctacagagatggcttcaacgtctccatcatgtataacctcactgttctgggtctggagcccccaactcccttgacagtgtacgctggagcaggttccagggtggggctgccctgccgcctgcctgctggtgtggggacccggtctttcctcactgccaagtggactcctcctgggggaggccctgacctcctggtgactggagacaatggcgactttacccttcgactagaggatgtgagccaggcccaggctgggacctacacctgccatatccatctgcaggaacagcagctcaatgccactgtcacattggcaatcatcacagtgactcccaaatcctttgggtcacctggatccctggggaagctgctttgtgaggtgactccagtatctggacaagaacgctttgtgtggagctctctggacaccccatcccagaggagtttctcaggaccttggctggaggcacaggaggcccagctcctttcccagccttggcaatgccagctgtaccagggggagaggcttcttggagcagcagtgtacttcacagagctgtctagcccaggtgcccaacgctctgggagagccccaggtgccctcccagcaggccacctcatccttgtgatcttctctggaatgttccttgttttcaccctggccggggccctgttcctccatcaacgaaggaaatatagatcaaacaaaggagaaagtcctgtggagcctgcagagccttgtcgttacagctgccccagggaggaggagggcagcaccatccccatccaggaggattaccgaaaaccggagcctgcctgctccccctga

SEQ ID NO: 47 - DNA, 1464 bp, Artificial Sequence
  Synthetic Construct
ctccagccaggggctgaggtcccggtggtgtgggcccaggagggggctcctgcccagctcccctgcagccccacaatccccctccaggatctcagccttctgcgaagagcaggggtcacttggcagcatcagccagacagtggcccgcccgctgccgcccccggccatcccctggcccccggccctcacccggcggcgccctcctcctgggggcccaggccccgccgctacacggtgctgagcgtgggtcccggaggcctgcgcagcgggaggctgcccctgcagccccgcgtccagctggatgagcgcggccggcagcgcggggacttctcgctatggctgcgcccagcccggcgcgcggacgccggcgagtaccgcgccgcggtgcacctcagggaccgcgccctctcctgccgcctccgtctgcgcctgggccaggcctcgatgactgccagccccccaggatctctcagagcctccgactgggtcattttgaactgctccttcagccgccctgaccgcccagcctctgtgcattggttccggaaccggggccagggccgagtccctgtccgggagtccccccatcaccacttagcggaaagcttcctcttcctgccccaagtcagccccatggactctgggccctggggctgcatcctcacctacagagatggcttcaacgtctccatcatgtataacctcactgttctgggtctggagcccccaactcccttgacagtgtacgctggagcaggttccagggtggggctgccctgccgcctgcctgctggtgtggggacccggtctttcctcactgccaagtggactcctcctgggggaggccctgacctcctggtgactggagacaatggcgactttacccttcgactagaggatgtgagccaggcccaggctgggacctacacctgccatatccatctgcaggaacagcagctcaatgccactgtcacattggcaatcatcacagtgactcccaaatcctttgggtcacctggatccctggggaagctgctttgtgaggtgactccagtatctggacaagaacgctttgtgtggagctctctggacaccccatcccagaggagtttctcaggaccttggctggaggcacaggaggcccagctcctttcccagccttggcaatgccagctgtaccagggggagaggcttcttggagcagcagtgtacttcacagagctgtctagcccaggtgcccaacgctctgggagagccccaggtgccctcccagcaggccacctcttctggttacccataggatgtgcagcctttgttgtagtctgcattttgggatgcatacttatttgttggcttacaaaaaagaagtattcatccagtgtgcacgaccctaacggtgaatacatgttcatgagagcagtgaacacagccaaaaaatctagactcacagatgtgaccctataa

SEQ ID NO: 48 - DNA, 1476 bp, Artificial Sequence
  Synthetic Construct
ctccagccaggggctgaggtcccggtggtgtgggcccaggagggggctcctgcccagctcccctgcagccccacaatccccctccaggatctcagccttctgcgaagagcaggggtcacttggcagcatcagccagacagtggcccgcccgctgccgcccccggccatcccctggcccccggccctcacccggcggcgccctcctcctgggggcccaggccccgccgctacacggtgctgagcgtgggtcccggaggcctgcgcagcgggaggctgcccctgcagccccgcgtccagctggatgagcgcggccggcagcgcggggacttctcgctatggctgcgcccagcccggcgcgcggacgccggcgagtaccgcgccgcggtgcacctcagggaccgcgccctctcctgccgcctccgtctgcgcctgggccaggcctcgatgactgccagccccccaggatctctcagagcctccgactgggtcattttgaactgctccttcagccgccctgaccgcccagcctctgtgcattggttccggaaccggggccagggccgagtccctgtccgggagtccccccatcaccacttagcggaaagcttcctcttcctgccccaagtcagccccatggactctgggccctggggctgcatcctcacctacagagatggcttcaacgtctccatcatgtataacctcactgttctgggtctggagcccccaactcccttgacagtgtacgctggagcaggttccagggtggggctgccctgccgcctgcctgctggtgtggggacccggtctttcctcactgccaagtggactcctcctgggggaggccctgacctcctggtgactggagacaatggcgactttacccttcgactagaggatgtgagccaggcccaggctgggacctacacctgccatatccatctgcaggaacagcagctcaatgccactgtcacattggcaatcatcacagtgactcccaaatcctttgggtcacctggatccctggggaagctgctttgtgaggtgactccagtatctggacaagaacgctttgtgtggagctctctggacaccccatcccagaggagtttctcaggaccttggctggaggcacaggaggcccagctcctttcccagccttggcaatgccagctgtaccagggggagaggcttcttggagcagcagtgtacttcacagagctgtctagcccaggtgcccaacgctctgggagagccccaggtgccctcccagcaggccacctcgttgccgccatcctgggcctgggcctggtgctggggctgctgggccccctggccatcctgctggccctgtacctgctccggagggaccagaggctgccccccgatgcccacaagccccctgggggaggcagtttccggacccccatccaagaggagcaggccgacgcccactccaccctggccaagatctga

SEQ ID NO: 49 - DNA, 1059 bp, Artificial Sequence
  Synthetic Construct
accccggaggtgtgggttcaagttcggatggaggccaccgagctctcgtccttcaccatccgttgtgggttcctggggtctggctccatctccctggtgactgtgagctgggggggccccaacggtgctggggggaccacgctggctgtgttgcacccagaacgtggcatccggcaatgggcccctgctcgccaggcccgctgggaaacccagagcagcatctctctcatcctggaaggctctggggccagcagcccctgcgccaacaccaccttctgctgcaagtttgcgtccttccctgagggctcctgggaggcctgtgggagcctcccgcccagctcagacccagggctctctgccccgccgactcctgcccccattctgcgggcagacctgcctatcttactaaccatcagcattttgagttttttctctgtcgctctgttggtcatcttggcctgtgtgttatggaaaaaaaggattaagcctatcgtatggcccagtctccccgatcataagaagactctggaacatctttgtaagaaaccaagaaaaaatttaaatgtgagtttcaatcctgaaagtttcctggactgccagattcatagggtggatgacattcaagctagagatgaagtggaaggttttctgcaagatacgtttcctcagcaactagaagaatctgagaagcagaggcttggaggggatgtgcagagccccaactgcccatctgaggatgtagtcgtcactccagaaagctttggaagagattcatccctcacatgcctggctgggaatgtcagtgcatgtgacgcccctattctctcctcttccaggtccctagactgcagggagagtggcaagaatgggcctcatgtgtaccaggacctcctgcttagccttgggactacaaacagcacgctgccccctccattttctctccaatctggaatcctgacattgaacccagttgctcagggtcagcccattcttacttccctgggatcaaatcaagaagaagcatatgtcaccatgtccagcttctaccaaaaccagtga

SEQ ID NO: 50 - DNA, 1332 bp, Artificial Sequence
  Synthetic Construct
accccggaggtgtgggttcaagttcggatggaggccaccgagctctcgtccttcaccatccgttgtgggttcctggggtctggctccatctccctggtgactgtgagctgggggggccccaacggtgctggggggaccacgctggctgtgttgcacccagaacgtggcatccggcaatgggcccctgctcgccaggcccgctgggaaacccagagcagcatctctctcatcctggaaggctctggggccagcagcccctgcgccaacaccaccttctgctgcaagtttgcgtccttccctgagggctcctgggaggcctgtgggagcctcccgcccagctcagacccagggctctctgccccgccgactcctgcccccattctgcgggcagacctgattccgtggctcggccacctcctcgtgggcctcagcggggcttttggcttcatcatcttagtgtacttgctgatcaactgcaggaacaccgggccatggctgaagaaggtcctgaagtgtaacaccccagacccctcgaagttcttttcccagctgagctcagagcatggaggagacgtccagaagtggctctcttcgcccttcccctcatcgtccttcagccctggcggcctggcacctgagatctcgccactagaagtgctggagagggacaaggtgacgcagctgctcctgcagcaggacaaggtgcctgagcccgcatccttaagcagcaaccactcgctgaccagctgcttcaccaaccagggttacttcttcttccacctcccggatgccttggagatagaggcctgccaggtgtactttacttacgacccctactcagaggaagaccctgatgagggtgtggccggggcacccacagggtcttccccccaacccctgcagcctctgtcaggggaggacgacgcctactgcaccttcccctccagggatgacctgctgctcttctcccccagtctcctcggtggccccagccccccaagcactgcccctgggggcagtggggccggtgaagagaggatgcccccttctttgcaagaaagagtccccagagactgggacccccagcccctggggcctcccaccccaggagtcccagacctggtggattttcagccaccccctgagctggtgctgcgagaggctggggaggaggtccctgacgctggccccagggagggagtcagtttcccctggtccaggcctcctgggcagggggagttcagggcccttaatgctcgcctgcccctgaacactgatgcctacttgtccctccaagaactccagggtcaggacccaactcacttggtgtag

SEQ ID NO: 51 - DNA, 1320 bp, Artificial Sequence
  Synthetic Construct
accccggaggtgtgggttcaagttcggatggaggccaccgagctctcgtccttcaccatccgttgtgggttcctggggtctggctccatctccctggtgactgtgagctgggggggccccaacggtgctggggggaccacgctggctgtgttgcacccagaacgtggcatccggcaatgggcccctgctcgccaggcccgctgggaaacccagagcagcatctctctcatcctggaaggctctggggccagcagcccctgcgccaacaccaccttctgctgcaagtttgcgtccttccctgagggctcctgggaggcctgtgggagcctcccgcccagctcagacccagggctctctgccccgccgactcctgcccccattctgcgggcagacctgggctggaaccctcacctgctgcttctcctcctgcttgtcatagtcttcattcctgccttctggagcctgaagacccatccattgtggaggctatggaagaagatatgggccgtccccagccctgagcggttcttcatgcccctgtacaagggctgcagcggagacttcaagaaatgggtgggtgcacccttcactggctccagcctggagctgggaccctggagcccagaggtgccctccaccctggaggtgtacagctgccacccaccacggagcccggccaagaggctgcagctcacggagctacaagaaccagcagagctggtggagtctgacggtgtgcccaagcccagcttctggccgacagcccagaactcggggggctcagcttacagtgaggagagggatcggccatacggcctggtgtccattgacacagtgactgtgctagatgcagaggggccatgcacctggccctgcagctgtgaggatgacggctacccagccctggacctggatgctggcctggagcccagcccaggcctagaggacccactcttggatgcagggaccacagtcctgtcctgtggctgtgtctcagctggcagccctgggctaggagggcccctgggaagcctcctggacagactaaagccaccccttgcagatggggaggactgggctgggggactgccctggggtggccggtcacctggaggggtctcagagagtgaggcgggctcacccctggccggcctggatatggacacgtttgacagtggctttgtgggctctgactgcagcagccctgtggagtgtgacttcaccagccccggggacgaaggacccccccggagctacctccgccagtgggtggtcattcctccgccactttcgagccctggaccccaggccagctaatga

SEQ ID NO: 52 - DNA, 1119 bp, Artificial Sequence
  Synthetic Construct
accccggaggtgtgggttcaagttcggatggaggccaccgagctctcgtccttcaccatccgttgtgggttcctggggtctggctccatctccctggtgactgtgagctgggggggccccaacggtgctggggggaccacgctggctgtgttgcacccagaacgtggcatccggcaatgggcccctgctcgccaggcccgctgggaaacccagagcagcatctctctcatcctggaaggctctggggccagcagcccctgcgccaacaccaccttctgctgcaagtttgcgtccttccctgagggctcctgggaggcctgtgggagcctcccgcccagctcagacccagggctctctgccccgccgactcctgcccccattctgcgggcagacctgtggatggcgtttgtggcaccaagcatttgcattgctatcatcatggtgggcattttctcaacgcattacttccagcaaaaggtgtttgttctcctagcagccctcagacctcagtggtgtagcagagaaattccagatccagcaaatagcacttgcgctaagaaatatcccattgcagaggagaagacacagctgcccttggacaggctcctgatagactggcccacgcctgaagatcctgaaccgctggtcatcagtgaagtccttcatcaagtgaccccagttttcagacatcccccctgctccaactggccacaaagggaaaaaggaatccaaggtcatcaggcctctgagaaagacatgatgcacagtgcctcaagcccaccacctccaagagctctccaagctgagagcagacaactggtggatctgtacaaggtgctggagagcaggggctccgacccaaagccagaaaacccagcctgtccctggacggtgctcccagcaggtgaccttcccacccatgatggctacttaccctccaacatagatgacctcccctcacatgaggcacctctcgctgactctctggaagaactggagcctcagcacatctccctttctgttttcccctcaagttctcttcacccactcaccttctcctgtggtgataagctgactctggatcagttaaagatgaggtgtgactccctcatgctctga

SEQ ID NO: 53 - DNA, 1294 bp, Artificial Sequence
  Synthetic Construct
accccggaggtgtgggttcaagttcggatggaggccaccgagctctcgtccttcaccatccgttgtgggttcctggggtctggctccatctccctggtgactgtgagctgggggggccccaacggtgctggggggaccacgctggctgtgttgcacccagaacgtggcatccggcaatgggcccctgctcgccaggcccgctgggaaacccagagcagcatctctctcatcctggaaggctctggggccagcagcccctgcgccaacaccaccttctgctgcaagtttgcgtccttccctgagggctcctgggaggcctgtgggagcctcccgcccagctcagacccagggctctctgccccgccgactcctgcccccattctgcgggcagacctggccatagtcgtgcctgtttgcttagcattcctattgacaactcttctgggagtgctgttctgctttaataagcgagacctaattaaaaaacacatctggcctaatgttccagatccttcaaagagtcatattgcccagtggtcacctcacactcctccaaggcacaattttaattcaaaagatcaaatgtattcagatggcaatttcactgatgtaagtgttgtggaaatagaagcaaatgacaaaaagccttttccagaagatctgaaatcattggacctgttcaaaaaggaaaaaattaatactgaaggacacagcagtggtattggggggtcttcatgcatgtcatcttctaggccaagcatttctagcagtgatgaaaatgaatcttcacaaaacacttcgagcactgtccagtattctaccgtggtacacagtggctacagacaccaagttccgtcagtccaagtcttctcaagatccgagtctacccagcccttgttagattcagaggagcggccagaagatctacaattagtagatcatgtagatggcggtgatggtattttgcccaggcaacagtacttcaaacagaactgcagtcagcatgaatccagtccagatatttcacattttgaaaggtcaaagcaagtttcatcagtcaatgaggaagattttgttagacttaaacagcagatttcagatcatatttcacaatcctgtggatctgggcaaatgaaaatgtttcaggaagtttctgcagcagatgcttttggtccaggtactgagggacaagtagaaagatttgaaacagttggcatggaggctgcgactgatgaaggcatgcctaaaagttacttaccacagactgtacggcaaggcggctacatgcctcagt

SEQ ID NO: 54 - DNA, 606 bp, Artificial Sequence
  Synthetic Construct
accccggaggtgtgggttcaagttcggatggaggccaccgagctctcgtccttcaccatccgttgtgggttcctggggtctggctccatctccctggtgactgtgagctgggggggccccaacggtgctggggggaccacgctggctgtgttgcacccagaacgtggcatccggcaatgggcccctgctcgccaggcccgctgggaaacccagagcagcatctctctcatcctggaaggctctggggccagcagcccctgcgccaacaccaccttctgctgcaagtttgcgtccttccctgagggctcctgggaggcctgtgggagcctcccgcccagctcagacccagggctctctgccccgccgactcctgcccccattctgcgggcagacctgatccttgtgatcttctctggaatgttccttgttttcaccctggccggggccctgttcctccatcaacgaaggaaatatagatcaaacaaaggagaaagtcctgtggagcctgcagagccttgtcgttacagctgccccagggaggaggagggcagcaccatccccatccaggaggattaccgaaaaccggagcctgcctgctccccctga

SEQ ID NO: 55 - DNA, 576 bp, Artificial Sequence
  Synthetic Construct
accccggaggtgtgggttcaagttcggatggaggccaccgagctctcgtccttcaccatccgttgtgggttcctggggtctggctccatctccctggtgactgtgagctgggggggccccaacggtgctggggggaccacgctggctgtgttgcacccagaacgtggcatccggcaatgggcccctgctcgccaggcccgctgggaaacccagagcagcatctctctcatcctggaaggctctggggccagcagcccctgcgccaacaccaccttctgctgcaagtttgcgtccttccctgagggctcctgggaggcctgtgggagcctcccgcccagctcagacccagggctctctgccccgccgactcctgcccccattctgcgggcagacctgttctggttacccataggatgtgcagcctttgttgtagtctgcattttgggatgcatacttatttgttggcttacaaaaaagaagtattcatccagtgtgcacgaccctaacggtgaatacatgttcatgagagcagtgaacacagccaaaaaatctagactcacagatgtgaccctataa

SEQ ID NO: 56 - DNA, 588 bp, Artificial Sequence
  Synthetic Construct
accccggaggtgtgggttcaagttcggatggaggccaccgagctctcgtccttcaccatccgttgtgggttcctggggtctggctccatctccctggtgactgtgagctgggggggccccaacggtgctggggggaccacgctggctgtgttgcacccagaacgtggcatccggcaatgggcccctgctcgccaggcccgctgggaaacccagagcagcatctctctcatcctggaaggctctggggccagcagcccctgcgccaacaccaccttctgctgcaagtttgcgtccttccctgagggctcctgggaggcctgtgggagcctcccgcccagctcagacccagggctctctgccccgccgactcctgcccccattctgcgggcagacctggttgccgccatcctgggcctgggcctggtgctggggctgctgggccccctggccatcctgctggccctgtacctgctccggagggaccagaggctgccccccgatgcccacaagccccctgggggaggcagtttccggacccccatccaagaggagcaggccgacgcccactccaccctggccaagatctga

SEQ ID NO: 57 - DNA, 1128 bp, Artificial Sequence
  Synthetic Construct
atcacccaacaagacctagctccccagcagagagcggccccacaacaaaagaggtccagcccctcagagggattgtgtccacctggacaccatatctcagaagacggtagagattgcatctcctgcaaatatggacaggactatagcactcactggaatgacctccttttctgcttgcgctgcaccaggtgtgattcaggtgaagtggagctaagtccgtgcaccacgaccagaaacacagtgtgtcagtgcgaagaaggcaccttccgggaagaagattctcctgagatgtgccggaagtgccgcacagggtgtcccagagggatggtcaaggtcggtgattgtacaccctggagtgacatcgaatgtgtccacaaagaatcaggtacaaagcacagtggggaagccccagctgtggaggagacggtgacctccagcccagggactcctgcctctccctgttctcctatcttactaaccatcagcattttgagttttttctctgtcgctctgttggtcatcttggcctgtgtgttatggaaaaaaaggattaagcctatcgtatggcccagtctccccgatcataagaagactctggaacatctttgtaagaaaccaagaaaaaatttaaatgtgagtttcaatcctgaaagtttcctggactgccagattcatagggtggatgacattcaagctagagatgaagtggaaggttttctgcaagatacgtttcctcagcaactagaagaatctgagaagcagaggcttggaggggatgtgcagagccccaactgcccatctgaggatgtagtcgtcactccagaaagctttggaagagattcatccctcacatgcctggctgggaatgtcagtgcatgtgacgcccctattctctcctcttccaggtccctagactgcagggagagtggcaagaatgggcctcatgtgtaccaggacctcctgcttagccttgggactacaaacagcacgctgccccctccattttctctccaatctggaatcctgacattgaacccagttgctcagggtcagcccattcttacttccctgggatcaaatcaagaagaagcatatgtcaccatgtccagcttctaccaaaaccagtga

SEQ ID NO: 58 - DNA, 1401 bp, Artificial Sequence
  Synthetic Construct
atcacccaacaagacctagctccccagcagagagcggccccacaacaaaagaggtccagcccctcagagggattgtgtccacctggacaccatatctcagaagacggtagagattgcatctcctgcaaatatggacaggactatagcactcactggaatgacctccttttctgcttgcgctgcaccaggtgtgattcaggtgaagtggagctaagtccgtgcaccacgaccagaaacacagtgtgtcagtgcgaagaaggcaccttccgggaagaagattctcctgagatgtgccggaagtgccgcacagggtgtcccagagggatggtcaaggtcggtgattgtacaccctggagtgacatcgaatgtgtccacaaagaatcaggtacaaagcacagtggggaagccccagctgtggaggagacggtgacctccagcccagggactcctgcctctccctgttctattccgtggctcggccacctcctcgtgggcctcagcggggcttttggcttcatcatcttagtgtacttgctgatcaactgcaggaacaccgggccatggctgaagaaggtcctgaagtgtaacaccccagacccctcgaagttcttttcccagctgagctcagagcatggaggagacgtccagaagtggctctcttcgcccttcccctcatcgtccttcagccctggcggcctggcacctgagatctcgccactagaagtgctggagagggacaaggtgacgcagctgctcctgcagcaggacaaggtgcctgagcccgcatccttaagcagcaaccactcgctgaccagctgcttcaccaaccagggttacttcttcttccacctcccggatgccttggagatagaggcctgccaggtgtactttacttacgacccctactcagaggaagaccctgatgagggtgtggccggggcacccacagggtcttccccccaacccctgcagcctctgtcaggggaggacgacgcctactgcaccttcccctccagggatgacctgctgctcttctcccccagtctcctcggtggccccagccccccaagcactgcccctgggggcagtggggccggtgaagagaggatgcccccttctttgcaagaaagagtccccagagactgggacccccagcccctggggcctcccaccccaggagtcccagacctggtggattttcagccaccccctgagctggtgctgcgagaggctggggaggaggtccctgacgctggccccagggagggagtcagtttcccctggtccaggcctcctgggcagggggagttcagggcccttaatgctcgcctgcccctgaacactgatgcctacttgtccctccaagaactccagggtcaggacccaactcacttggtgtag

SEQ ID NO: 59 - DNA, 1389 bp, Artificial Sequence
  Synthetic Construct
atcacccaacaagacctagctccccagcagagagcggccccacaacaaaagaggtccagcccctcagagggattgtgtccacctggacaccatatctcagaagacggtagagattgcatctcctgcaaatatggacaggactatagcactcactggaatgacctccttttctgcttgcgctgcaccaggtgtgattcaggtgaagtggagctaagtccgtgcaccacgaccagaaacacagtgtgtcagtgcgaagaaggcaccttccgggaagaagattctcctgagatgtgccggaagtgccgcacagggtgtcccagagggatggtcaaggtcggtgattgtacaccctggagtgacatcgaatgtgtccacaaagaatcaggtacaaagcacagtggggaagccccagctgtggaggagacggtgacctccagcccagggactcctgcctctccctgttctggctggaaccctcacctgctgcttctcctcctgcttgtcatagtcttcattcctgccttctggagcctgaagacccatccattgtggaggctatggaagaagatatgggccgtccccagccctgagcggttcttcatgcccctgtacaagggctgcagcggagacttcaagaaatgggtgggtgcacccttcactggctccagcctggagctgggaccctggagcccagaggtgccctccaccctggaggtgtacagctgccacccaccacggagcccggccaagaggctgcagctcacggagctacaagaaccagcagagctggtggagtctgacggtgtgcccaagcccagcttctggccgacagcccagaactcggggggctcagcttacagtgaggagagggatcggccatacggcctggtgtccattgacacagtgactgtgctagatgcagaggggccatgcacctggccctgcagctgtgaggatgacggctacccagccctggacctggatgctggcctggagcccagcccaggcctagaggacccactcttggatgcagggaccacagtcctgtcctgtggctgtgtctcagctggcagccctgggctaggagggcccctgggaagcctcctggacagactaaagccaccccttgcagatggggaggactgggctgggggactgccctggggtggccggtcacctggaggggtctcagagagtgaggcgggctcacccctggccggcctggatatggacacgtttgacagtggctttgtgggctctgactgcagcagccctgtggagtgtgacttcaccagccccggggacgaaggacccccccggagctacctccgccagtgggtggtcattcctccgccactttcgagccctggaccccaggccagctaatga

SEQ ID NO: 60 - DNA, 1188 bp, Artificial Sequence
  Synthetic Construct
atcacccaacaagacctagctccccagcagagagcggccccacaacaaaagaggtccagcccctcagagggattgtgtccacctggacaccatatctcagaagacggtagagattgcatctcctgcaaatatggacaggactatagcactcactggaatgacctccttttctgcttgcgctgcaccaggtgtgattcaggtgaagtggagctaagtccgtgcaccacgaccagaaacacagtgtgtcagtgcgaagaaggcaccttccgggaagaagattctcctgagatgtgccggaagtgccgcacagggtgtcccagagggatggtcaaggtcggtgattgtacaccctggagtgacatcgaatgtgtccacaaagaatcaggtacaaagcacagtggggaagccccagctgtggaggagacggtgacctccagcccagggactcctgcctctccctgttcttggatggcgtttgtggcaccaagcatttgcattgctatcatcatggtgggcattttctcaacgcattacttccagcaaaaggtgtttgttctcctagcagccctcagacctcagtggtgtagcagagaaattccagatccagcaaatagcacttgcgctaagaaatatcccattgcagaggagaagacacagctgcccttggacaggctcctgatagactggcccacgcctgaagatcctgaaccgctggtcatcagtgaagtccttcatcaagtgaccccagttttcagacatcccccctgctccaactggccacaaagggaaaaaggaatccaaggtcatcaggcctctgagaaagacatgatgcacagtgcctcaagcccaccacctccaagagctctccaagctgagagcagacaactggtggatctgtacaaggtgctggagagcaggggctccgacccaaagccagaaaacccagcctgtccctggacggtgctcccagcaggtgaccttcccacccatgatggctacttaccctccaacatagatgacctcccctcacatgaggcacctctcgctgactctctggaagaactggagcctcagcacatctccctttctgttttcccctcaagttctcttcacccactcaccttctcctgtggtgataagctgactctggatcagttaaagatgaggtgtgactccctcatgctctga

SEQ ID NO: 61 - DNA, 1363 bp, Artificial Sequence
  Synthetic Construct
atcacccaacaagacctagctccccagcagagagcggccccacaacaaaagaggtccagcccctcagagggattgtgtccacctggacaccatatctcagaagacggtagagattgcatctcctgcaaatatggacaggactatagcactcactggaatgacctccttttctgcttgcgctgcaccaggtgtgattcaggtgaagtggagctaagtccgtgcaccacgaccagaaacacagtgtgtcagtgcgaagaaggcaccttccgggaagaagattctcctgagatgtgccggaagtgccgcacagggtgtcccagagggatggtcaaggtcggtgattgtacaccctggagtgacatcgaatgtgtccacaaagaatcaggtacaaagcacagtggggaagccccagctgtggaggagacggtgacctccagcccagggactcctgcctctccctgttctgccatagtcgtgcctgtttgcttagcattcctattgacaactcttctgggagtgctgttctgctttaataagcgagacctaattaaaaaacacatctggcctaatgttccagatccttcaaagagtcatattgcccagtggtcacctcacactcctccaaggcacaattttaattcaaaagatcaaatgtattcagatggcaatttcactgatgtaagtgttgtggaaatagaagcaaatgacaaaaagccttttccagaagatctgaaatcattggacctgttcaaaaaggaaaaaattaatactgaaggacacagcagtggtattggggggtcttcatgcatgtcatcttctaggccaagcatttctagcagtgatgaaaatgaatcttcacaaaacacttcgagcactgtccagtattctaccgtggtacacagtggctacagacaccaagttccgtcagtccaagtcttctcaagatccgagtctacccagcccttgttagattcagaggagcggccagaagatctacaattagtagatcatgtagatggcggtgatggtattttgcccaggcaacagtacttcaaacagaactgcagtcagcatgaatccagtccagatatttcacattttgaaaggtcaaagcaagtttcatcagtcaatgaggaagattttgttagacttaaacagcagatttcagatcatatttcacaatcctgtggatctgggcaaatgaaaatgtttcaggaagtttctgcagcagatgcttttggtccaggtactgagggacaagtagaaagatttgaaacagttggcatggaggctgcgactgatgaaggcatgcctaaaagttacttaccacagactgtacggcaaggcggctacatgcctcagt

SEQ ID NO: 62 - DNA, 675 bp, Artificial Sequence
  Synthetic Construct
atcacccaacaagacctagctccccagcagagagcggccccacaacaaaagaggtccagcccctcagagggattgtgtccacctggacaccatatctcagaagacggtagagattgcatctcctgcaaatatggacaggactatagcactcactggaatgacctccttttctgcttgcgctgcaccaggtgtgattcaggtgaagtggagctaagtccgtgcaccacgaccagaaacacagtgtgtcagtgcgaagaaggcaccttccgggaagaagattctcctgagatgtgccggaagtgccgcacagggtgtcccagagggatggtcaaggtcggtgattgtacaccctggagtgacatcgaatgtgtccacaaagaatcaggtacaaagcacagtggggaagccccagctgtggaggagacggtgacctccagcccagggactcctgcctctccctgttctatccttgtgatcttctctggaatgttccttgttttcaccctggccggggccctgttcctccatcaacgaaggaaatatagatcaaacaaaggagaaagtcctgtggagcctgcagagccttgtcgttacagctgccccagggaggaggagggcagcaccatccccatccaggaggattaccgaaaaccggagcctgcctgctccccctga

SEQ ID NO: 63 - DNA, 645 bp, Artificial Sequence
  Synthetic Construct
atcacccaacaagacctagctccccagcagagagcggccccacaacaaaagaggtccagcccctcagagggattgtgtccacctggacaccatatctcagaagacggtagagattgcatctcctgcaaatatggacaggactatagcactcactggaatgacctccttttctgcttgcgctgcaccaggtgtgattcaggtgaagtggagctaagtccgtgcaccacgaccagaaacacagtgtgtcagtgcgaagaaggcaccttccgggaagaagattctcctgagatgtgccggaagtgccgcacagggtgtcccagagggatggtcaaggtcggtgattgtacaccctggagtgacatcgaatgtgtccacaaagaatcaggtacaaagcacagtggggaagccccagctgtggaggagacggtgacctccagcccagggactcctgcctctccctgttctttctggttacccataggatgtgcagcctttgttgtagtctgcattttgggatgcatacttatttgttggcttacaaaaaagaagtattcatccagtgtgcacgaccctaacggtgaatacatgttcatgagagcagtgaacacagccaaaaaatctagactcacagatgtgaccctataa

SEQ ID NO: 64 - DNA, 654 bp, Artificial Sequence
  Synthetic Construct
atcacccaacaagacctagctccccagcagagagcggccccacaacaaaagaggtccagcccctcagagggattgtgtccacctggacaccatatctcagaagacggtagagattgcatctcctgcaaatatggacaggactatagcactcactggaatgacctccttttctgcttgcgctgcaccaggtgtgattcaggtgaagtggagctaagtccgtgcaccacgaccagaaacacagtgtgtcagtgcgaagaaggcaccttccgggaagaagattctcctgagatgtgccggaagtgccgcacagggtgtcccagagggatggtcaaggtcggtgattgtacaccctggagtgacatcgaatgtgtccacaaagaatcaggtacaaagcacagtggggaagccccagctgtggaggagacggtgacctccagcccagggactcctgcctctccctgttctgttgccgccatcctgggcctgggcctggtgctggggctgctgggccccctggccatcctgctggccctgtacctgctccggagggaccagaggctgccccccgatgcccacaagccccctgggggaggcagtttccggacccccatccaagaggagcaggccgacgcccactccaccctggccaagatc

SEQ ID NO: 65 - protein, 181 aa, Artificial Sequence
  Synthetic Construct
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTLQRDFTAAFPRMLTTRGHGPAETQTLGSLPDINLTQISTLANELRDSRLANDLRDSGATIRIG

SEQ ID NO: 66 - protein, 428 aa, Artificial Sequence
  Synthetic Construct
LQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHL

SEQ ID NO: 67 - protein, 132 aa, Artificial Sequence
  Synthetic Construct
TPEVWVQVRMEATELSSFTIRCGFLGSGSISLVTVSWGGPNGAGGTTLAVLHPERGIRQWAPARQARWETQSSISLILEGSGASSPCANTTFCCKFASFPEGSWEACGSLPPSSDPGLSAPPTPAPILRADL

SEQ ID NO: 68 - protein, 155 aa, Artificial Sequence
  Synthetic Construct
ITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASPCS

SEQ ID NO: 69 - protein, 220 aa, Artificial Sequence
  Synthetic Construct
PILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 70 - protein, 311 aa, Artificial Sequence
  Synthetic Construct
IPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 71 - protein, 306 aa, Artificial Sequence
  Synthetic Construct
GWNPHLLLLLLLVIVFIPAFWSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 72 - protein, 240 aa, Artificial Sequence
  Synthetic Construct
WMAFVAPSICIAIIMVGIFSTHYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 73 - protein, 299 aa, Artificial Sequence
  Synthetic Construct
AIVVPVCLAFLLTTLLGVLFCFNKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 74 - protein, 5 aa, Artificial Sequence
  Synthetic Construct
NYGMN

SEQ ID NO: 75 - protein, 5 aa, Artificial Sequence
  Synthetic Construct
GYAMS

SEQ ID NO: 76 - protein, 6 aa, Artificial Sequence
  Synthetic Construct
  MISC_FEATURE (1)..(1): Xaa is Arg or Gly
  MISC_FEATURE (3)..(3): Xaa is Ala or Gly
  misc_feature (4)..(4): Xaa can be any naturally occurring amino acid
XYAXMS

SEQ ID NO: 77 - protein, 17 aa, Artificial Sequence
  Synthetic Construct
  MISC_FEATURE (12)..(12): Xaa is Ala or Val
  MISC_FEATURE (13)..(13): Xaa is Gly or Asp
WINTYTGEPTYXXDFKG

SEQ ID NO: 78 - protein, 17 aa, Artificial Sequence
  Synthetic Construct
TISSGGTYIYYPDSVKG

SEQ ID NO: 79 - protein, 9 aa, Artificial Sequence
  Synthetic Construct
  MISC_FEATURE (1)..(1): Xaa is Ser or Thr
  MISC_FEATURE (3)..(3): Xaa is Thr or Asp
  MISC_FEATURE (4)..(4): Xaa is Thr or Ala
  MISC_FEATURE (5)..(6): Xaa is any amino acid
  MISC_FEATURE (7)..(7): Xaa is Tyr or Phe
XGXXXXXYA

SEQ ID NO: 80 - protein, 9 aa, Artificial Sequence
  Synthetic Construct
TGTTAILNG

SEQ ID NO: 81 - protein, 9 aa, Artificial Sequence
  Synthetic Construct
SGDGYWYYA

SEQ ID NO: 82 - protein, 10 aa, Artificial Sequence
  Synthetic Construct
  MISC_FEATURE (5)..(6): Xaa is any amino acid
  MISC_FEATURE (10)..(10): Xaa is Gly or Ala
DNYGXXYDYX

SEQ ID NO: 83 - protein, 10 aa, Artificial Sequence
  Synthetic Construct
  MISC_FEATURE (6)..(6): Xaa is Val or Ile
  MISC_FEATURE (9)..(9): Xaa is Met or Ile
  MISC_FEATURE (10)..(10): Xaa is His or Tyr
SASSSXSYXX

SEQ ID NO: 84 - protein, 15 aa, Artificial Sequence
  Synthetic Construct
RASKSVSTSGYSYMH

SEQ ID NO: 85 - protein, 7 aa, Artificial Sequence
  Synthetic Construct
  MISC_FEATURE (1)..(1): Xaa is Ser or Gly
XTSNLAS

SEQ ID NO: 86 - protein, 7 aa, Artificial Sequence
  Synthetic Construct
LASNLES

SEQ ID NO: 87 - protein, 9 aa, Artificial Sequence
  Synthetic Construct
  MISC_FEATURE (5)..(5): Xaa is Ser or Asn
  MISC_FEATURE (8)..(8): Xaa is Ser or Phe
QQRSXYPXT

SEQ ID NO: 88 - protein, 9 aa, Artificial Sequence
  Synthetic Construct
QHSRELPFT

SEQ ID NO: 89 - protein, 116 aa, Artificial Sequence
  Synthetic Construct
VQLQESGGGLVQPGGSMKLSCVASGFTFSNYWMNWVRQSPEKGLEWVAEIRLKSNNYATHYAESVKGRFTISRDDSKSSVYLQMNNLRAEDTGIYYCTGVGQFAYWGQGTTVTVSS

SEQ ID NO: 90 - protein, 106 aa, Artificial Sequence
  Synthetic Construct
DIELTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKL

SEQ ID NO: 91 - protein, 106 aa, Artificial Sequence; identical to SEQ ID NO: 90
  Synthetic Construct

SEQ ID NO: 92 - protein, 121 aa, Artificial Sequence
  Synthetic Construct
GGGGSVQLQESGGGLVQPGGSMKLSCVASGFTFSNYWMNWVRQSPEKGLEWVAEIRLKSNNYATHYAESVKGRFTISRDDSKSSVYLQMNNLRAEDTGIYYCTGVGQFAYWGQGTTVTVSS

SEQ ID NO: 93 - protein, 237 aa, Artificial Sequence
  Synthetic Construct
VQLQESGGGLVQPGGSMKLSCVASGFTFSNYWMNWVRQSPEKGLEWVAEIRLKSNNYATHYAESVKGRFTISRDDSKSSVYLQMNNLRAEDTGIYYCTGVGQFAYWGQGTTVTVSSGGGGSGGGGSGGGGSDIELTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKL

SEQ ID NO: 94 - protein, 243 aa, Artificial Sequence
  Synthetic Construct
EIVLTQSPATLSLSPGERATLTCSATSSVSYIHWYQQRPGQSPRLLIYSTSNLASGIPARFSGSGSGSDYTLTISSLEPEDFAVYYCQQRSSSPFTFGSGTKVEIKGGGGSGGGGSGGGGSEVQLVESGGGLVKPGGSLRLSCAASGFTFSRYGMSWVRQAPGKRLEWVSTISGGGTYIYYPDSVKGRFTISRDNAKNTLYLQMNSLRAEDTAVYYCTRDNYGRNYDYGMDYWGQGTLVTVSS

SEQ ID NO: 95 - DNA, 1578 bp, Artificial Sequence
  Synthetic Construct
atgtgggaggctcagttcctgggcttgctgtttctgcagccgctttgggtggctccagtgaagcctctccagccaggggctgaggtcccggtggtgtgggcccaggagggggctcctgcccagctcccctgcagccccacaatccccctccaggatctcagccttctgcgaagagcaggggtcacttggcagcatcagccagacagtggcccgcccgctgccgcccccggccatcccctggcccccggccctcacccggcggcgccctcctcctgggggcccaggccccgccgctacacggtgctgagcgtgggtcccggaggcctgcgcagcgggaggctgcccctgcagccccgcgtccagctggatgagcgcggccggcagcgcggggacttctcgctatggctgcgcccagcccggcgcgcggacgccggcgagtaccgcgccgcggtgcacctcagggaccgcgccctctcctgccgcctccgtctgcgcctgggccaggcctcgatgactgccagccccccaggatctctcagagcctccgactgggtcattttgaactgctccttcagccgccctgaccgcccagcctctgtgcattggttccggaaccggggccagggccgagtccctgtccgggagtccccccatcaccacttagcggaaagcttcctcttcctgccccaagtcagccccatggactctgggccctggggctgcatcctcacctacagagatggcttcaacgtctccatcatgtataacctcactgttctgggtctggagcccccaactcccttgacagtgtacgctggagcaggttccagggtggggctgccctgccgcctgcctgctggtgtggggacccggtctttcctcactgccaagtggactcctcctgggggaggccctgacctcctggtgactggagacaatggcgactttacccttcgactagaggatgtgagccaggcccaggctgggacctacacctgccatatccatctgcaggaacagcagctcaatgccactgtcacattggcaatcatcacagtgactcccaaatcctttgggtcacctggatccctggggaagctgctttgtgaggtgactccagtatctggacaagaacgctttgtgtggagctctctggacaccccatcccagaggagtttctcaggaccttggctggaggcacaggaggcccagctcctttcccagccttggcaatgccagctgtaccagggggagaggcttcttggagcagcagtgtacttcacagagctgtctagcccaggtgcccaacgctctgggagagccccaggtgccctcccagcaggccacctcctgctgtttctcacccttggtgtcctttctctgctccttttggtgactggagcctttggctttcacctttggagaagacagtggcgaccaagacgattttctgccttagagcaagggattcaccctccgcaggctcagagcaagatagaggagctggagcaagaaccggagccggagccggagccggaaccggagcccgagcccgagcccgagccggagcagctctga

SEQ ID NO: 96 - DNA, 981 bp, Artificial Sequence
  Synthetic Construct
atgagaacagaggcacaggtgccggccctgcagcccccagaacctggactggagggggccatggggcaccggaccctggtcctgccctgggtgctgctgaccttgtgtgtcactgcggggaccccggaggtgtgggttcaagttcggatggaggccaccgagctctcgtccttcaccatccgttgtgggttcctggggtctggctccatctccctggtgactgtgagctgggggggccccaacggtgctggggggaccacgctggctgtgttgcacccagaacgtggcatccggcaatgggcccctgctcgccaggcccgctgggaaacccagagcagcatctctctcatcctggaaggctctggggccagcagcccctgcgccaacaccaccttctgctgcaagtttgcgtccttccctgagggctcctgggaggcctgtgggagcctcccgcccagctcagacccagggctctctgccccgccgactcctgcccccattctgcgggcagacctggccgggatcttgggggtctcaggagtcctcctctttggctgtgtctacctccttcatctgctgcgccgacataagcaccgccctgcccctaggctccagccgtcccgcaccagcccccaggcaccgagagcacgagcatgggcaccaagccaggcctcccaggctgctcttcacgtcccttatgccactatcaacaccagctgccgcccagctactttggacacagctcacccccatggggggccgtcctggtgggcgtcactccccacccacgctgcacaccggccccagggccctgccgcctgggcctccacacccatccctgcacgtggcagctttgtctctgttgagaatggactctacgctcaggcaggggagaggcctcctcacactggtcccggcctcactcttttccctgaccctcgggggcccagggccatggaaggacccttaggagttcgatga

SEQ ID NO: 97 - DNA, 735 bp, Artificial Sequence
  Synthetic Construct
atgcgctggtgtctcctcctgatctgggcccaggggctgaggcaggctcccctcgcctcaggaatgatgacaggcacaatagaaacaacggggaacatttctgcagagaaaggtggctctatcatcttacaatgtcacctctcctccaccacggcacaagtgacccaggtcaactgggagcagcaggaccagcttctggccatttgtaatgctgacttggggtggcacatctccccatccttcaaggatcgagtggccccaggtcccggcctgggccttaccctccagtcgctgaccgtgaacgatacaggggagtacttctgcatctatcacacctaccctgatggggcgtacactgggagaatcttcctggaggtcctagaaagctcagtggctgagcacggtgccaggttccagattccattgcttggagccatggccgcgacgctggtggtcatctgcacagcagtcatcgtggtggtcgcgttgactagaaagaagaaagccctcagaatccattctgtggaaggtgacctcaggagaaaatcagctggacaggaggaatggagccccagtgctccctcacccccaggaagctgtgtccaggcagaagctgcacctgctgggctctgtggagagcagcggggagaggactgtgccgagctgcatgactacttcaatgtcctgagttacagaagcctgggtaactgcagcttcttcacagagactggttag

SEQ ID NO: 98 - DNA, 906 bp, Artificial Sequence
  Synthetic Construct
atgttttcacatcttccctttgactgtgtcctgctgctgctgctgctactacttacaaggtcctcagaagtggaatacagagcggaggtcggtcagaatgcctatctgccctgcttctacaccccagccgccccagggaacctcgtgcccgtctgctggggcaaaggagcctgtcctgtgtttgaatgtggcaacgtggtgctcaggactgatgaaagggatgtgaattattggacatccagatactggctaaatggggatttccgcaaaggagatgtgtccctgaccatagagaatgtgattctagcagacagtgggatctactgctgccggatccaaatcccaggcataatgaatgatgaaaaatttaacctgaagttggtcatcaaaccagccaaggtcacccctgcaccgactctgcagagagacttcactgcagcctttccaaggatgcttaccaccaggggacatggcccagcagagacacagacactggggagcctccctgatataaatctaacacaaatatccacattggccaatgagttacgggactctagattggccaatgacttacgggactctggagcaaccatcagaataggcatctacatcggagcagggatctgtgctgggctggctctggctcttatcttcggcgctttaattttcaaatggtattctcatagcaaagagaagatacagaatttaagcctcatctctttggccaacctccctccctcaggattggcaaatgcagtagcagagggaattcgctcagaagaaaacatctataccattgaagagaacgtatatgaagtggaggagcccaatgagtattattgctatgtcagcagcaggcagcaaccctcacaacctttgggttgtcgctttgcaatgccatag

SEQ ID NO: 99 - DNA, 1323 bp, Artificial Sequence
  Synthetic Construct
atggaacaacggggacagaacgccccggccgcttcgggggcccggaaaaggcacggcccaggacccagggaggcgcggggagccaggcctgggccccgggtccccaagacccttgtgctcgttgtcgccgcggtcctgctgttggtctcagctgagtctgctctgatcacccaacaagacctagctccccagcagagagcggccccacaacaaaagaggtccagcccctcagagggattgtgtccacctggacaccatatctcagaagacggtagagattgcatctcctgcaaatatggacaggactatagcactcactggaatgacctccttttctgcttgcgctgcaccaggtgtgattcaggtgaagtggagctaagtccgtgcaccacgaccagaaacacagtgtgtcagtgcgaagaaggcaccttccgggaagaagattctcctgagatgtgccggaagtgccgcacagggtgtcccagagggatggtcaaggtcggtgattgtacaccctggagtgacatcgaatgtgtccacaaagaatcaggtacaaagcacagtggggaagccccagctgtggaggagacggtgacctccagcccagggactcctgcctctccctgttctctctcaggcatcatcataggagtcacagttgcagccgtagtcttgattgtggctgtgtttgtttgcaagtctttactgtggaagaaagtccttccttacctgaaaggcatctgctcaggtggtggtggggaccctgagcgtgtggacagaagctcacaacgacctggggctgaggacaatgtcctcaatgagatcgtgagtatcttgcagcccacccaggtccctgagcaggaaatggaagtccaggagccagcagagccaacaggtgtcaacatgttgtcccccggggagtcagagcatctgctggaaccggcagaagctgaaaggtctcagaggaggaggctgctggttccagcaaatgaaggtgatcccactgagactctgagacagtgcttcgatgactttgcagacttggtgccctttgactcctgggagccgctcatgaggaagttgggcctcatggacaatgagataaaggtggctaaagctgaggcagcgggccacagggacaccttgtacacgatgctgataaagtgggtcaacaaaaccgggcgagatgcctctgtccacaccctgctggatgccttggagacgctgggagagagacttgccaagcagaagattgaggaccacttgttgagctctggaaagttcatgtatctagaaggtaatgcagactctgccatgtcctaa

SEQ ID NO: 100 - protein, 22 aa, Artificial Sequence
  Synthetic Construct
GSGATNFSLLKQAGDVEENPGP

SEQ ID NO: 101 - DNA, 66 bp, Artificial Sequence
  Synthetic Construct
ggcagcggcgccaccaacttcagcctgctgaagcaggccggcgacgtggaggagaaccccggcccc

SEQ ID NO: 102 - protein, 21 aa, Artificial Sequence
  Synthetic Construct
MALPVTALLLPLALLLHAARP

SEQ ID NO: 103 - DNA, 63 bp, Artificial Sequence
  Synthetic Construct
atggccttaccagtgaccgccttgctcctgccgctggccttgctgctccacgccgccaggccg

SEQ ID NO: 104 - protein, 444 aa, Artificial Sequence
  Synthetic Construct
GSGATNFSLLKQAGDVEENPGPMALPVTALLLPLALLLHAARPSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTLQRDFTAAFPRMLTTRGHGPAETQTLGSLPDINLTQISTLANELRDSRLANDLRDSGATIRIGPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 105 - DNA, 1332 bp, Artificial Sequence
  Synthetic Construct
ggcagcggcgccaccaacttcagcctgctgaagcaggccggcgacgtggaggagaaccccggccccatggccttaccagtgaccgccttgctcctgccgctggccttgctgctccacgccgccaggccgtcagaagtggaatacagagcggaggtcggtcagaatgcctatctgccctgcttctacaccccagccgccccagggaacctcgtgcccgtctgctggggcaaaggagcctgtcctgtgtttgaatgtggcaacgtggtgctcaggactgatgaaagggatgtgaattattggacatccagatactggctaaatggggatttccgcaaaggagatgtgtccctgaccatagagaatgtgactctagcagacagtgggatctactgctgccggatccaaatcccaggcataatgaatgatgaaaaatttaacctgaagttggtcatcaaaccagccaaggtcacccctgcaccgactctgcagagagacttcactgcagcctttccaaggatgcttaccaccaggggacatggcccagcagagacacagacactggggagcctccctgatataaatctaacacaaatatccacattggccaatgagttacgggactctagattggccaatgacttacgggactctggagcaaccatcagaataggccctatcttactaaccatcagcattttgagttttttctctgtcgctctgttggtcatcttggcctgtgtgttatggaaaaaaaggattaagcctatcgtatggcccagtctccccgatcataagaagactctggaacatctttgtaagaaaccaagaaaaaatttaaatgtgagtttcaatcctgaaagtttcctggactgccagattcatagggtggatgacattcaagctagagatgaagtggaaggttttctgcaagatacgtttcctcagcaactagaagaatctgagaagcagaggcttggaggggatgtgcagagccccaactgcccatctgaggatgtagtcgtcactccagaaagctttggaagagattcatccctcacatgcctggctgggaatgtcagtgcatgtgacgcccctattctctcctcttccaggtccctagactgcagggagagtggcaagaatgggcctcatgtgtaccaggacctcctgcttagccttgggactacaaacagcacgctgccccctccattttctctccaatctggaatcctgacattgaacccagttgctcagggtcagcccattcttacttccctgggatcaaatcaagaagaagcatatgtcaccatgtccagcttctaccaaaaccag

SEQ ID NO: 106 - protein, 534 aa, Artificial Sequence
  Synthetic Construct
GSGATNFSLLKQAGDVEENPGPMALPVTALLLPLALLLHAARPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLVAAILGLGLVLGLLGPLAILLALYLLRRDQRLPPDAHKPPGGGSFRTPIQEEQADAHSTLAKI

SEQ ID NO: 107 - DNA, 1602 bp, Artificial Sequence
  Synthetic Construct
ggcagcggcgccaccaacttcagcctgctgaagcaggccggcgacgtggaggagaaccccggccccatggccttaccagtgaccgccttgctcctgccgctggccttgctgctccacgccgccaggccgctccagccaggggctgaggtcccggtggtgtgggcccaggagggggctcctgcccagctcccctgcagccccacaatccccctccaggatctcagccttctgcgaagagcaggggtcacttggcagcatcagccagacagtggcccgcccgctgccgcccccggccatcccctggcccccggccctcacccggcggcgccctcctcctgggggcccaggccccgccgctacacggtgctgagcgtgggtcccggaggcctgcgcagcgggaggctgcccctgcagccccgcgtccagctggatgagcgcggccggcagcgcggggacttctcgctatggctgcgcccagcccggcgcgcggacgccggcgagtaccgcgccgcggtgcacctcagggaccgcgccctctcctgccgcctccgtctgcgcctgggccaggcctcgatgactgccagccccccaggatctctcagagcctccgactgggtcattttgaactgctccttcagccgccctgaccgcccagcctctgtgcattggttccggaaccggggccagggccgagtccctgtccgggagtccccccatcaccacttagcggaaagcttcctcttcctgccccaagtcagccccatggactctgggccctggggctgcatcctcacctacagagatggcttcaacgtctccatcatgtataacctcactgttctgggtctggagcccccaactcccttgacagtgtacgctggagcaggttccagggtggggctgccctgccgcctgcctgctggtgtggggacccggtctttcctcactgccaagtggactcctcctgggggaggccctgacctcctggtgactggagacaatggcgactttacccttcgactagaggatgtgagccaggcccaggctgggacctacacctgccatatccatctgcaggaacagcagctcaatgccactgtcacattggcaatcatcacagtgactcccaaatcctttgggtcacctggatccctggggaagctgctttgtgaggtgactccagtatctggacaagaacgctttgtgtggagctctctggacaccccatcccagaggagtttctcaggaccttggctggaggcacaggaggcccagctcctttcccagccttggcaatgccagctgtaccagggggagaggcttcttggagcagcagtgtacttcacagagctgtctagcccaggtgcccaacgctctgggagagccccaggtgccctcccagcaggccacctcgttgccgccatcctgggcctgggcctggtgctggggctgctgggccccctggccatcctgctggccctgtacctgctccggagggaccagaggctgccccccgatgcccacaagccccctgggggaggcagtttccggacccccatccaagaggagcaggccgacgcccactccaccctggccaagatc

SEQ ID NO: 108 - protein, 486 aa, Artificial Sequence
  Synthetic Construct
GSGATNFSLLKQAGDVEENPGPMALPVTALLLPLALLLHAARPTPEVWVQVRMEATELSSFTIRCGFLGSGSISLVTVSWGGPNGAGGTTLAVLHPERGIRQWAPARQARWETQSSISLILEGSGASSPCANTTFCCKFASFPEGSWEACGSLPPSSDPGLSAPPTPAPILRADLIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 109 - DNA, 1458 bp, Artificial Sequence
  Synthetic Construct
ggcagcggcgccaccaacttcagcctgctgaagcaggccggcgacgtggaggagaaccccggccccatggccttaccagtgaccgccttgctcctgccgctggccttgctgctccacgccgccaggccgaccccggaggtgtgggttcaagttcggatggaggccaccgagctctcgtccttcaccatccgttgtgggttcctggggtctggctccatctccctggtgactgtgagctgggggggccccaacggtgctggggggaccacgctggctgtgttgcacccagaacgtggcatccggcaatgggcccctgctcgccaggcccgctgggaaacccagagcagcatctctctcatcctggaaggctctggggccagcagcccctgcgccaacaccaccttctgctgcaagtttgcgtccttccctgagggctcctgggaggcctgtgggagcctcccgcccagctcagacccagggctctctgccccgccgactcctgcccccattctgcgggcagacctgattccgtggctcggccacctcctcgtgggcctcagcggggcttttggcttcatcatcttagtgtacttgctgatcaactgcaggaacaccgggccatggctgaagaaggtcctgaagtgtaacaccccagacccctcgaagttcttttcccagctgagctcagagcatggaggagacgtccagaagtggctctcttcgcccttcccctcatcgtccttcagccctggcggcctggcacctgagatctcgccactagaagtgctggagagggacaaggtgacgcagctgctcctgcagcaggacaaggtgcctgagcccgcatccttaagcagcaaccactcgctgaccagctgcttcaccaaccagggttacttcttcttccacctcccggatgccttggagatagaggcctgccaggtgtactttacttacgacccctactcagaggaagaccctgatgagggtgtggccggggcacccacagggtcttccccccaacccctgcagcctctgtcaggggaggacgacgcctactgcaccttcccctccagggatgacctgctgctcttctcccccagtctcctcggtggccccagccccccaagcactgcccctgggggcagtggggccggtgaagagaggatgcccccttctttgcaagaaagagtccccagagactgggacccccagcccctggggcctcccaccccaggagtcccagacctggtggattttcagccaccccctgagctggtgctgcgagaggctggggaggaggtccctgacgctggccccagggagggagtcagtttcccctggtccaggcctcctgggcagggggagttcagggcccttaatgctcgcctgcccctgaacactgatgcctacttgtccctccaagaactccagggtcaggacccaactcacttggtg

SEQ ID NO: 110 - protein, 10 aa, Artificial Sequence
  Synthetic Construct
GFTFSNYWMN

SEQ ID NO: 111 - protein, 14 aa, Artificial Sequence
  Synthetic Construct
RLKSNNYATHYAES

SEQ ID NO: 112 - protein, 6 aa, Artificial Sequence
  Synthetic Construct
VGQFAY

SEQ ID NO: 113 - protein, 12 aa, Artificial Sequence
  Synthetic Construct
STGAVTTSNYAN

SEQ ID NO: 114 - protein, 7 aa, Artificial Sequence
  Synthetic Construct
GTNNRAP

SEQ ID NO: 115 - protein, 9 aa, Artificial Sequence
  Synthetic Construct
ALWYSNHWV

SEQ ID NO: 116 - protein, 218 aa, Artificial Sequence
  Synthetic Construct
QVQLQESGGGLVQPGGSMKLSCVASGFTFSNYWMNWVRQSPEKGLEWVAEIRLKSNNYATHYAESVKGRFTISRDDSKSSVYLQMNNLRAEDTGIYYCTGVGQFAYWGQGTTVTVSSAKTTPPTVYPLAPGSNAASQSMVTLGCLVKGYFPEPVTVTWNSGSLASGVHTFPAVLQSDLYTLSSSVTVPSSTWPSETVTCNVAHPASSTKVDAKIVPRD

SEQ ID NO: 117 - protein, 215 aa, Artificial Sequence
  Synthetic Construct
DIVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGSEKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 118 - protein, 9 aa, Artificial Sequence
  Synthetic Construct
YTFTDHAIH

SEQ ID NO: 119 - protein, 14 aa, Artificial Sequence
  Synthetic Construct
WIGHFSPGNTDIKY

SEQ ID NO: 120 - protein, 8 aa, Artificial Sequence
  Synthetic Construct
KTSTFFFD

SEQ ID NO: 121 - protein, 13 aa, Artificial Sequence
  Synthetic Construct
SQSLLNSGDQKNY

SEQ ID NO: 122 - protein, 10 aa, Artificial Sequence
  Synthetic Construct
KLLIFWASTR

SEQ ID NO: 123 - protein, 7 aa, Artificial Sequence
  Synthetic Construct
CQNDYSY

SEQ ID NO: 124 - protein, 115 aa, Artificial Sequence
  Synthetic Construct
VQLQQSDAELVKPGSSVKISCKASGYTFTDHAIHWVKQKPEQGLEWIGHFSPGNTDIKYNDKFKGKATLTVDRSSSTAYMQLNSLTSEDSAVYFCKTSTFFFDYWGQGTTLTVSS

SEQ ID NO: 125 - protein, 113 aa, Artificial Sequence
  Synthetic Construct
ELVMTQSPSSLTVTAGEKVTMICKSSQSLLNSGDQKNYLTWYQQKPGQPPKLLIFWASTRESGVPDRFTGSGSGTDFTLTISSVQAEDLAVYYCQNDYSYPLTFGAGTKLELK

SEQ ID NO: 126 - protein, 244 aa, Artificial Sequence
  Synthetic Construct
QVQLQQSDAELVKPGSSVKISCKASGYTFTDHAIHWVKQKPEQGLEWIGHFSPGNTDIKYNDKFKGKATLTVDRSSSTAYMQLNSLTSEDSAVYFCKTSTFFFDYWGQGTTLTVSSGGGGSGGGGSGGGGSELVMTQSPSSLTVTAGEKVTMICKSSQSLLNSGDQKNYLTWYQQKPGQPPKLLIFWASTRESGVPDRFTGSGSGTDFTLTISSVQAEDLAVYYCQNDYSYPLTFGAGTKLELK

SEQ ID NO: 127 - protein, 524 aa, Artificial Sequence
  Synthetic Construct
MLRLLLALNLFPSIQVTGQVQLQQSDAELVKPGSSVKISCKASGYTFTDHAIHWVKQKPEQGLEWIGHFSPGNTDIKYNDKFKGKATLTVDRSSSTAYMQLNSLTSEDSAVYFCKTSTFFFDYWGQGTTLTVSSGGGGSGGGGSGGGGSELVMTQSPSSLTVTAGEKVTMICKSSQSLLNSGDQKNYLTWYQQKPGQPPKLLIFWASTRESGVPDRFTGSGSGTDFTLTISSVQAEDLAVYYCQNDYSYPLTFGAGTKLELKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 128 - protein, 768 aa, Artificial Sequence
  Synthetic Construct
MLRLLLALNLFPSIQVTGQVQLQQSDAELVKPGSSVKISCKASGYTFTDHAIHWVKQKPEQGLEWIGHFSPGNTDIKYNDKFKGKATLTVDRSSSTAYMQLNSLTSEDSAVYFCKTSTFFFDYWGQGTTLTVSSGGGGSGGGGSGGGGSELVMTQSPSSLTVTAGEKVTMICKSSQSLLNSGDQKNYLTWYQQKPGQPPKLLIFWASTRESGVPDRFTGSGSGTDFTLTISSVQAEDLAVYYCQNDYSYPLTFGAGTKLELKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGSGMVSKGEELFTGVVPILVELDGDVNGHKFSVRGEGEGDATNGKLTLKFICTSGKLPVPWPTLVTTLSYGVQCFARYPDHMKQHDFFKSAMPEGYVQERTISFKDDGSYRTRAEVKFEGDTLVNRIELKGIDFKEDGNILGHKLEYNMNVWDAYITADKQKNGIKANFKIEHNVEDGGVQLADAYQQNTPIGDGSVLLPDNHYLSFQSKLFKDPNEQRDHMVLLEFVTAAGITPGMDELYK

SEQ ID NO: 129 - protein, 1010 aa, Artificial Sequence
  Synthetic Construct
MLRLLLALNLFPSIQVTGQVQLQQSDAELVKPGSSVKISCKASGYTFTDHAIHWVKQKPEQGLEWIGHFSPGNTDIKYNDKFKGKATLTVDRSSSTAYMQLNSLTSEDSAVYFCKTSTFFFDYWGQGTTLTVSSGGGGSGGGGSGGGGSELVMTQSPSSLTVTAGEKVTMICKSSQSLLNSGDQKNYLTWYQQKPGQPPKLLIFWASTRESGVPDRFTGSGSGTDFTLTISSVQAEDLAVYYCQNDYSYPLTFGAGTKLELKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPTPEVWVQVRMEATELSSFTIRCGFLGSGSISLVTVSWGGPNGAGGTTLAVLHPERGIRQWAPARQARWETQSSISLILEGSGASSPCANTTFCCKFASFPEGSWEACGSLPPSSDPGLSAPPTPAPILRADLIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

(19) United States
(12) Patent Application Publication
(10) Pub. No.: US 2024/0269281 A1
(43) Pub. Date: Aug. 15, 2024

(54) CAR-T CELL THERAPY FOR TRIPLE NEGATIVE BREAST CANCER

(71) Applicant: H. LEE MOFFITT CANCER CENTER AND RESEARCH INSTITUTE, INC., Tampa, FL (US)

(72) Inventors: Patrick HWU, Tampa, FL (US); Narges TAFRESHI, Tampa, FL (US); Soraya Zorro MANRIQUE, Tampa, FL (US); Reginald ATKINS, Apollo Beach, FL (US); Yunfei WANG, Houston, TX (US)

(21) Appl. No.: 18/565,805

(22) PCT Filed: Jun. 22, 2022
(86) PCT No.: PCT/US2022/073079

§ 371 Date: Nov. 30, 2023

Related U.S. Application Data
(60) Provisional application No. 63/213,960, filed on Jun. 23, 2021.
(60) Provisional application No. 63/214,433, filed on Jun. 24, 2021.
(60) Provisional application No. 63/332,060, filed on Apr. 18, 2022.

(51) Int. Cl. A61K 39/00; C07K 14/54; C07K 14/705; C07K 14/715; C07K 16/30; C12N 5/0783

(52) U.S. Cl. CPC A61K 39/4611; A61K 39/4631; A61K 39/46447; C07K 14/5443; C07K 14/70503; C07K 14/70521; C07K 14/70578; C07K 14/7155; C07K 16/3092; C12N 5/0636; A61K 2239/15; A61K 2239/49; C07K 2319/02; C07K 2319/03; C12N 2510/00

(57) ABSTRACT

As disclosed herein, high expression of the inhibitory ligands Galectin-3 (LAG-3 ligand), Galectin-9 (Tim-3 ligand), HMGB1 (Tim-3 ligand), CD112 (CD112R ligand) and TNFSF10 (DR5 ligand) in the microenvironment of triple negative breast cancer cells is inhibitory for T-cells. Therefore, disclosed herein is a chimeric receptor comprising an extracellular domain of TIM-3, LAG-3, CD112R or DR5 and transmembrane and intracellular domain of a pro-inflammatory interleukin and/or a co-stimulatory domain. Also disclosed is an immune effector cell engineered to express a chimeric antigen receptor (CAR) polypeptide and the disclosed chimeric receptor. Also disclosed is a method of providing an anti-cancer immunity in a subject with a MUC1-expressing cancer, the method comprising administering to the subject an effective amount of the disclosed immune effector cell, thereby providing an anti-tumor immunity in the subject.

### CROSS-REFERENCE TO RELATED APPLICATIONS

This application claims benefit of U.S. Provisional Application No. 63/213,960, filed Jun. 23, 2021, U.S. Provisional Application No. 63/214,433, filed Jun. 24, 2021, and U.S. Provisional Application No. 63/332,060, filed Apr. 18, 2022, which are hereby incorporated herein by reference in their entireties.

### SEQUENCE LISTING

This application contains a sequence listing filed in electronic form as an ASCII.txt file entitled “320803_2830_Sequence_Listing_ST25” created on Jun. 20, 2022, having 268,053 bytes. The content of the sequence listing is incorporated herein in its entirety.

### BACKGROUND

In women worldwide, breast cancer has the highest occurrence among all malignancies (Damasceno, M., Curr Opin Oncol, 2011. 23 Suppl: S3-9). About 75% of breast cancers are negative for the human epidermal growth factor receptor 2 (HER2). A subgroup of these patients are also negative for hormone receptors, and are thus triple-negative (ER, PR and HER2). In addition to the triple negative ductal carcinomas, the rare and aggressive metaplastic breast cancers are also commonly triple negative, and are generally refractory to chemotherapy and radiation; this rare but particularly aggressive histology is observed in less than 5% of all breast cancer cases (Leyrer, C. M., et al., Breast Cancer Res Treat, 2017; El Zein, D., et al., Clin Breast Cancer, 2017. 17(5):382-391). The triple negative subtype, regardless of histology, is associated with increased risk of local-regional and distant recurrence compared to hormone-responsive or Her2neu-overexpressing subtypes (El Zein, D., et al., Clin Breast Cancer, 2017. 17(5):382-391; Giuliano, A. E., et al., CA: A Cancer Journal for Clinicians, 2017. 67(4):290-303). Most triple negative breast tumors are resistant, or eventually become resistant, to existing cytotoxic chemotherapy; this is evidenced by the relatively low rate of complete tumor eradication among triple negative breast cancer patients receiving preoperative (neoadjuvant) chemotherapy (Andre, F. et al., Ann Oncol, 2012. 23 Suppl 6:vi46-51; Geyer, F. C., et al., Am J Pathol, 2017; Carey, L. A., Breast, 2017; Chen, V. E., et al., Adv Radiat Oncol, 2017. 2(2):105-109). In fact, women receiving neoadjuvant chemotherapy with evidence of residual tumor at the time of surgery are considered at significantly higher risk for relapse than women with a pathologic complete response (pCR) to preoperative chemotherapy, and recent data suggests that additional toxic systemic therapy may confer a survival benefit (Carey, L. A., Breast, 2017; Chen, V. E., et al., Adv Radiat Oncol, 2017. 2(2): 105-109; Masuda, N., et al., N Engl J Med, 2017. 376(22):2147-2159). In the case of metaplastic breast cancers, tumors are observed to progress even on systemic therapy or fail to demonstrate any response to therapy at the time of surgery (Leyrer, C. M., et al., Breast Cancer Res Treat, 2017; El Zein, D., et al., Clin Breast Cancer, 2017. 17(5):382-391). These aggressive treatment lines involve months of toxic preoperative therapy, and increasingly, the burden of additional post-surgical systemic therapy with additive toxicities and side effects. Thus, the development of novel therapeutic approaches for therapy-resistant triple negative breast cancer (TR-TNBC) is a significant unmet medical need, as there are limited effective therapies, even for patients with de novo disease.

### SUMMARY

As disclosed herein, high expression of the inhibitory ligands Galectin-3 (LAG-3 ligand), Galectin-9 (Tim-3 ligand), HMGB1 (Tim-3 ligand), CD112 (CD112R ligand) and TNFSF10 (DR5 ligand) in the microenvironment of triple negative breast cancer cells is inhibitory for T-cells. This means that if CAR T cells are not changed, they will not be able to work properly in this inhibitory tumor microenvironment. Therefore, three strategies were developed to manipulate the CAR-T cells.

The first strategy involved fusing the extracellular part of the inhibitory receptors (Tim-3, LAG3, CD112R and DR5) to transmembrane and intracellular part of the pro-inflammatory interleukins (IL7Rα, βcommon part of IL2 and IL15, IL21R, IL12Rb2, IL6ST) or co-stimulatory domains (CD27, ICOS, OX40).

The second strategy involves knocking down one or more of the inhibitory receptors (FIG. 1B), including LAG-3, CD112R, TIGIT, TIM3 and DR5 using shRNA in the same vector.

The third strategy would be combinational therapy using VEGFA or VEGFR antagonist antibodies, since the concentration of VEGFA is very high in TNBC microenvironment based on the TCGA RNA seq data.

Therefore, disclosed herein is a chimeric receptor comprising an extracellular domain of TIM-3, LAG-3, CD112R or DR5 and an intracellular domain of a pro-inflammatory interleukin and/or a co-stimulatory domain. In some embodiments, the pro-inflammatory interleukin is selected from the group consisting of IL7Rα, IL2Rβ, IL15, IL21R, IL12Rβ2 and IL6ST. In some embodiments, the co-stimulatory domain is selected from the group consisting of CD27, ICOS, OX40.

Also disclosed is an immune effector cell engineered to express a chimeric antigen receptor (CAR) polypeptide and the disclosed chimeric receptor. In some embodiments, the immune effector cell is selected from the group consisting of an αβT cell, γδT cell, a Natural Killer (NK) cells, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, a regulatory T cell, or any combination thereof.

A CAR is generally made up of three domains: an ectodomain, a transmembrane domain, and an endodomain. The ectodomain comprises an antigen-binding region and is responsible for antigen recognition. It also optionally contains a signal peptide (SP) so that the CAR can be glycosylated and anchored in the cell membrane of the immune effector cell. The transmembrane domain (TD), is as its name suggests, connects the ectodomain to the endodomain and resides within the cell membrane when expressed by a cell. The endodomain is the business end of the CAR that transmits an activation signal to the immune effector cell after antigen recognition. For example, the endodomain can contain an intracellular signaling domain (ISD) and optionally a co-stimulatory signaling region (CSR).

In some embodiments, the tumor antigen is any tumor antigen that is not expressed on healthy tissue. In some embodiments, the tumor antigen is MUC-1.

Also disclosed herein is an immune effector cell engineered to express a chimeric receptor disclosed herein, a chimeric antigen receptor (CAR) polypeptide, and engineered to silence expression of inhibitory receptors selected from the group consisting of TIM-3, LAG-3, CD112R, TIGIT and DR5.

In some embodiments, the immune effector cell is engineered to express a chimeric antigen receptor (CAR) polypeptide and engineered to silence expression of inhibitory receptors selected from the group consisting of TIM-3, LAG-3, CD112R, TIGIT and DR5.

In some embodiments, the disclosed chimeric receptor, CAR polypeptide, and/or shRNA are expressed in the immune effector cell using a retroviral vector. For example, in some embodiments, the vector is a pMXs (Cell Biolabs, Inc.) retroviral vector based on Moloney murine leukemia virus (MMLV). In some embodiments, the vector is a pDON-AI, pMEI-5 or pDON-5 (Takara Bio Inc.) retroviral vector contain only LTR and packaging signal (Ψ sequence) of MoMLV genome, but not gag, pol, and env coding sequence.

Also disclosed is a method of providing an anti-cancer immunity in a subject, the method comprising administering to the subject an effective amount of the disclosed immune effector cell, thereby providing an anti-tumor immunity in the subject. In some embodiments, the method further involves administering to the subject a checkpoint inhibitor, such as an anti-PD-1 antibody, anti-PD-L1 antibody, anti-CTLA-4 antibody, or a combination thereof. In some embodiments, the cancer is a triple negative breast cancer (TNBC).

In some embodiments, the disclosed method further involves administering to the subject a vascular endothelial growth factor-A (VEGF-A) or vascular endothelial growth factor receptor (VEGFR) antagonist antibody. VEGF inhibitors utilized in oncologic care include bevacizumab, sorafenib, sunitinib, nilotinib, pazopanib, and dasatinib.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A to 1B show engineering of CAR T cells to combat the inhibitory TNBC microenvironment by making chimeric receptor (FIG. 1A), knocking down one or more of the TIM-3, LAG-3, CD112R, TIGIT and DR5 (FIG. 1B), or combinational therapy using VEGFA or VEGFR antagonist antibodies (FIGS. 1A and 1B).

FIGS. 2A to 2J show quantification of the targets expression after multiplex immune fluorescent (mIF) staining on protein level on TNBC samples and normal tissues. FIGS. 2A to 2J show B7H4 (FIG. 2A), LAMP2 (FIG. 2B), TROP2 (FIG. 2C), Cadherin-3 (FIG. 2D), MUC1 (FIG. 2E), FR alpha (FIG. 2F), CDCP1 (FIG. 2G), SLC39A (FIG. 2H), EPHB3 (FIG. 2I), and GABRP (FIG. 2J) expression in TNBC and normal tissues. The multi-layer TIFF images from mIF were loaded onto HALO (Indica Labs) for segmentation and quantitative digital image analyses. For each marker, a positivity threshold within the nucleus or cytoplasm was determined per marker based on published staining patterns and intensity for that specific antibody.

FIG. 3A shows expression of inhibitory proteins in TNBC tumors. FIG. 3B shows immune cells population in TNBC. mIF was used for staining and the same method as FIG. 1 was used for quantification.

FIG. 4 illustrates a disclosed approach for designing the CAR-T constructs for Muc-1 and B7H4 with different features (i.e. CRISPR knock-out of inhibitory receptors, adding chimeric switch receptors to CAR) and combination with VEGFR/VEGFA antibodies. Chimeric receptors=switch receptors.

FIG. 5 shows expression of CD112 in different cancers. TGCT: Testicular Germ Cell Tumors, LUSC: Lung squamous cell carcinoma, SKCM: Skin Cutaneous Melanoma, KIRC: Kidney renal clear cell carcinoma, KICH: Kidney Chromophobe, THCA: Thyroid carcinoma, LUAD: Lung adenocarcinoma, ACC: Adrenocortical carcinoma, KIRP: Kidney renal papillary cell carcinoma, CESC: Cervical squamous cell carcinoma, and endocervical adenocarcinoma, ESCA: Esophageal carcinoma, UCS: Uterine CarcinosarcomaBLCA: Bladder Urothelial Carcinoma, PRAD: Prostate adenocarcinoma, LIHC: Liver hepatocellular carcinoma, LGG: Brain Lower Grade Glioma, COAD: Colon adenocarcinoma, UCEC: Uterine Corpus Endometrial Carcinoma, READ: Rectum adenocarcinoma, BRCA: Breast invasive carcinoma, DLBC: Lymphoid Neoplasm Diffuse Large B-cell Lymphoma, GBM: Glioblastoma multiforme, THYM: Thymoma, and PAAD: Pancreatic adenocarcinoma.

FIG. 6 illustrates a disclosed approach that involves adding switch chimeric receptor to CAR construct. There was a focus on the extracellular part of the CD112R with transmembrane and intracellular domain of IL2R2β. The CD8a was used for the signaling peptide of the chimeric switch receptor. The switch receptor is used to eliminate the negative signal (e.g. from CD112/CD112R) and converting to positive signal. Signal 3 provides survival, proliferation and persistence. The IL2R2β was used because IL2 is the most potent of the ILs for effector T-cells. The switch receptor has the potential of replacing with systemic IL-2 Infusion, decreasing cytokine release syndrome.

FIG. 7 illustrates an embodiment of a MUC1 CAR construct. 5E5 scfv was used to target Tn antigen of Muc-1. We called this construct Muc1-BB28z.

FIG. 8 illustrates an embodiment of a MUC1 CAR and CD112-IL2Rβ chimeric receptor construct. 5E5 scfv was used to target Tn antigen of Muc-1. The extracellular domain of CD112R was fused with intracellular domain of IL2Rβ. We called this construct Muc1-BB28z+CD112R^{ED}-IL2Rβ^{CSD}.

FIGS. 9A and 9B show expression of MUC1 and CD112 antigens on BT-549 (FIG. 9A) and MDA-mb-231 (FIG. 9B) cell lines using immunofluorescent staining and flow staining.

FIGS. 10A to 10D show real time Cytotoxicity Assay (RTCA) on BT549 cells which are high antigen (Muc-1) expressing cells using two different ratio of 1:1 and 1:5 on different healthy donors (HD).

FIGS. 11A and 11B show real time Cytotoxicity Assay (RTCA) on MDA-mb-231 cells which are low antigen (Muc-1) expressing cells using two different ratio of 1:1 and 1:5 on different healthy donors (HD).

FIG. 12 shows real time Cytotoxicity Assay (RTCA) on Jurkat Tn-cells which don't have the antigen (Muc-1) using ratio of 1:1 and 1:5 on different healthy donors (HD).

FIGS. 13A and 13B show quantification of pSTAT5 staining of T cells (UT is un-transduced cells) using flow; 15 min and 2 h after stimulation with target cells (BT-549 cells) in two different Healthy Donors (HDs).

FIGS. 14A to 14C show quantification of yellow tracer. The T cells were labeled with Yellow tracer and stimulated with target cells. The yellow tracer was quantified 72 h after stimulation.

FIGS. 15A to 15C show quantification of GRANB, IFNγ and TNF-α production in T-cells unstimulated and stimulated with target cells in HD3.

FIGS. 16A to 16C show quantification of GRANB, IFNγ and TNF-α production in T-cells unstimulated and stimulated with target cells in HD26.

FIGS. 17A and 17B show CD112R expression using flow staining in two different HD.

FIGS. 18A and 18B show CD4/CD8 ratio in two different HD.

FIGS. 19A and 19B show percentage of different T cells subtypes in CD4 and CD8 cells in HD1.

FIGS. 20A and 20B show percentage of different T cells subtypes in CD4 and CD8 cells in HD2.

FIGS. 21A and 21B show expression of exhaustion markers in CD4 and CD8 cells in HD1.

FIGS. 22A and 22B show expression of exhaustion markers in CD4 and CD8 cells in HD2.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

The term “amino acid sequence” refers to a list of abbreviations, letters, characters or words representing amino acid residues. The amino acid abbreviations used herein are conventional one letter codes for the amino acids and are expressed as follows: A, alanine; B, asparagine or aspartic acid; C, cysteine; D aspartic acid; E, glutamate, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine; Z, glutamine or glutamic acid.

The term “antibody” refers to an immunoglobulin, derivatives thereof which maintain specific binding ability, and proteins having a binding domain which is homologous or largely homologous to an immunoglobulin binding domain. These proteins may be derived from natural sources, or partly or wholly synthetically produced. An antibody may be monoclonal or polyclonal. The antibody may be a member of any immunoglobulin class from any species, including any of the human classes: IgG, IgM, IgA, IgD, and IgE. In exemplary embodiments, antibodies used with the methods and compositions described herein are derivatives of the IgG class. In addition to intact immunoglobulin molecules, also included in the term “antibodies” are fragments or polymers of those immunoglobulin molecules, and human or humanized versions of immunoglobulin molecules that selectively bind the target antigen.

The term “antibody fragment” refers to any derivative of an antibody which is less than full-length. In exemplary embodiments, the antibody fragment retains at least a significant portion of the full-length antibody's specific binding ability. Examples of antibody fragments include, but are not limited to, Fab, Fab′, F(ab′)2, scFv, Fv, dsFv diabody, Fc, and Fd fragments. The antibody fragment may be produced by any means. For instance, the antibody fragment may be enzymatically or chemically produced by fragmentation of an intact antibody, it may be recombinantly produced from a gene encoding the partial antibody sequence, or it may be wholly or partially synthetically produced. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment will typically comprise at least about 50 amino acids and more typically will comprise at least about 200 amino acids.

The term “antigen binding site” refers to a region of an antibody that specifically binds an epitope on an antigen.

The term “aptamer” refers to oligonucleic acid or peptide molecules that bind to a specific target molecule. These molecules are generally selected from a random sequence pool. The selected aptamers are capable of adapting unique tertiary structures and recognizing target molecules with high affinity and specificity. A “nucleic acid aptamer” is a DNA or RNA oligonucleic acid that binds to a target molecule via its conformation, and thereby inhibits or suppresses functions of such molecule. A nucleic acid aptamer may be constituted by DNA, RNA, or a combination thereof. A “peptide aptamer” is a combinatorial protein molecule with a variable peptide sequence inserted within a constant scaffold protein. Identification of peptide aptamers is typically performed under stringent yeast dihybrid conditions, which enhances the probability for the selected peptide aptamers to be stably expressed and correctly folded in an intracellular context.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “chimeric molecule” refers to a single molecule created by joining two or more molecules that exist separately in their native state. The single, chimeric molecule has the desired functionality of all of its constituent molecules. One type of chimeric molecules is a fusion protein.

The term “engineered antibody” refers to a recombinant molecule that comprises at least an antibody fragment comprising an antigen binding site derived from the variable domain of the heavy chain and/or light chain of an antibody and may optionally comprise the entire or part of the variable and/or constant domains of an antibody from any of the Ig classes (for example IgA, IgD, IgE, IgG, IgM and IgY).

The term “epitope” refers to the region of an antigen to which an antibody binds preferentially and specifically. A monoclonal antibody binds preferentially to a single specific epitope of a molecule that can be molecularly defined. In the present invention, multiple epitopes can be recognized by a multispecific antibody.

The term “fusion protein” refers to a polypeptide formed by the joining of two or more polypeptides through a peptide bond formed between the amino terminus of one polypeptide and the carboxyl terminus of another polypeptide. The fusion protein can be formed by the chemical coupling of the constituent polypeptides or it can be expressed as a single polypeptide from nucleic acid sequence encoding the single contiguous fusion protein. A single chain fusion protein is a fusion protein having a single contiguous polypeptide backbone. Fusion proteins can be prepared using conventional techniques in molecular biology to join the two genes in frame into a single nucleic acid, and then expressing the nucleic acid in an appropriate host cell under conditions in which the fusion protein is produced.

The term “Fab fragment” refers to a fragment of an antibody comprising an antigen-binding site generated by cleavage of the antibody with the enzyme papain, which cuts at the hinge region N-terminally to the inter-H-chain disulfide bond and generates two Fab fragments from one antibody molecule.

The term “F(ab′)2 fragment” refers to a fragment of an antibody containing two antigen-binding sites, generated by cleavage of the antibody molecule with the enzyme pepsin which cuts at the hinge region C-terminally to the inter-H-chain disulfide bond.

The term “Fc fragment” refers to the fragment of an antibody comprising the constant domain of its heavy chain.

The term “Fv fragment” refers to the fragment of an antibody comprising the variable domains of its heavy chain and light chain.

“Gene construct” refers to a nucleic acid, such as a vector, plasmid, viral genome or the like which includes a “coding sequence” for a polypeptide or which is otherwise transcribable to a biologically active RNA (e.g., antisense, decoy, ribozyme, etc), may be transfected into cells, e.g. in certain embodiments mammalian cells, and may cause expression of the coding sequence in cells transfected with the construct. The gene construct may include one or more regulatory elements operably linked to the coding sequence, as well as intronic sequences, polyadenylation sites, origins of replication, marker genes, etc.

The term “identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting. For example, polypeptides having at least 70%, 85%, 90%, 95%, 98% or 99% identity to specific polypeptides described herein and preferably exhibiting substantially the same functions, as well as polynucleotide encoding such polypeptides, are contemplated. Unless otherwise indicated a similarity score will be based on use of BLOSUM62. When BLASTP is used, the percent similarity is based on the BLASTP positives score and the percent sequence identity is based on the BLASTP identities score. BLASTP “Identities” shows the number and fraction of total residues in the high scoring sequence pairs which are identical; and BLASTP “Positives” shows the number and fraction of residues for which the alignment scores have positive values and which are similar to each other. Amino acid sequences having these degrees of identity or similarity or any intermediate degree of identity of similarity to the amino acid sequences disclosed herein are contemplated and encompassed by this disclosure. The polynucleotide sequences of similar polypeptides are deduced using the genetic code and may be obtained by conventional means, in particular by reverse translating its amino acid sequence using the genetic code.

The term “linker” is art-recognized and refers to a molecule or group of molecules connecting two compounds, such as two polypeptides. The linker may be comprised of a single linking molecule or may comprise a linking molecule and a spacer molecule, intended to separate the linking molecule and a compound by a specific distance.

The term “multivalent antibody” refers to an antibody or engineered antibody comprising more than one antigen recognition site. For example, a “bivalent” antibody has two antigen recognition sites, whereas a “tetravalent” antibody has four antigen recognition sites. The terms “monospecific”, “bispecific”, “trispecific”, “tetraspecific”, etc. refer to the number of different antigen recognition site specificities (as opposed to the number of antigen recognition sites) present in a multivalent antibody. For example, a “monospecific” antibody's antigen recognition sites all bind the same epitope. A “bispecific” antibody has at least one antigen recognition site that binds a first epitope and at least one antigen recognition site that binds a second epitope that is different from the first epitope. A “multivalent monospecific” antibody has multiple antigen recognition sites that all bind the same epitope. A “multivalent bispecific” antibody has multiple antigen recognition sites, some number of which bind a first epitope and some number of which bind a second epitope that is different from the first epitope.

The term “nucleic acid” refers to a natural or synthetic molecule comprising a single nucleotide or two or more nucleotides linked by a phosphate group at the 3′ position of one nucleotide to the 5′ end of another nucleotide. The nucleic acid is not limited by length, and thus the nucleic acid can include deoxyribonucleic acid (DNA) or ribonucleic acid (RNA).

The term “operably linked to” refers to the functional relationship of a nucleic acid with another nucleic acid sequence. Promoters, enhancers, transcriptional and translational stop sites, and other signal sequences are examples of nucleic acid sequences operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA.

The terms “peptide,” “protein,” and “polypeptide” are used interchangeably to refer to a natural or synthetic molecule comprising two or more amino acids linked by the carboxyl group of one amino acid to the alpha amino group of another.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The terms “polypeptide fragment” or “fragment”, when used in reference to a particular polypeptide, refers to a polypeptide in which amino acid residues are deleted as compared to the reference polypeptide itself, but where the remaining amino acid sequence is usually identical to that of the reference polypeptide. Such deletions may occur at the amino-terminus or carboxy-terminus of the reference polypeptide, or alternatively both. Fragments typically are at least about 5, 6, 8 or 10 amino acids long, at least about 14 amino acids long, at least about 20, 30, 40 or 50 amino acids long, at least about 75 amino acids long, or at least about 100, 150, 200, 300, 500 or more amino acids long. A fragment can retain one or more of the biological activities of the reference polypeptide. In various embodiments, a fragment may comprise an enzymatic activity and/or an interaction site of the reference polypeptide. In another embodiment, a fragment may have immunogenic properties.

The term “protein domain” refers to a portion of a protein, portions of a protein, or an entire protein showing structural integrity; this determination may be based on amino acid composition of a portion of a protein, portions of a protein, or the entire protein.

The term “single chain variable fragment or scFv” refers to an Fv fragment in which the heavy chain domain and the light chain domain are linked. One or more scFv fragments may be linked to other antibody fragments (such as the constant domain of a heavy chain or a light chain) to form antibody constructs having one or more antigen recognition sites.

A “spacer” as used herein refers to a peptide that joins the proteins comprising a fusion protein. Generally a spacer has no specific biological activity other than to join the proteins or to preserve some minimum distance or other spatial relationship between them. However, the constituent amino acids of a spacer may be selected to influence some property of the molecule such as the folding, net charge, or hydrophobicity of the molecule.

The term “specifically binds”, as used herein, when referring to a polypeptide (including antibodies) or receptor, refers to a binding reaction which is determinative of the presence of the protein or polypeptide or receptor in a heterogeneous population of proteins and other biologics. Thus, under designated conditions (e.g. immunoassay conditions in the case of an antibody), a specified ligand or antibody “specifically binds” to its particular “target” (e.g. an antibody specifically binds to an endothelial antigen) when it does not bind in a significant amount to other proteins present in the sample or to other proteins to which the ligand or antibody may come in contact in an organism. Generally, a first molecule that “specifically binds” a second molecule has an affinity constant (Ka) greater than about 10⁵ M⁻¹ (e.g., 10⁶ M⁻¹, 10⁷ M⁻¹, 10⁸ M⁻¹, 10⁹ M⁻¹, 10¹⁰ M⁻¹, 10¹¹ M⁻¹, and 10¹² M⁻¹ or more) with that second molecule.

The term “specifically deliver” as used herein refers to the preferential association of a molecule with a cell or tissue bearing a particular target molecule or marker and not to cells or tissues lacking that target molecule. It is, of course, recognized that a certain degree of non-specific interaction may occur between a molecule and a non-target cell or tissue. Nevertheless, specific delivery, may be distinguished as mediated through specific recognition of the target molecule. Typically specific delivery results in a much stronger association between the delivered molecule and cells bearing the target molecule than between the delivered molecule and cells lacking the target molecule.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The terms “transformation” and “transfection” mean the introduction of a nucleic acid, e.g., an expression vector, into a recipient cell including introduction of a nucleic acid to the chromosomal DNA of said cell.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “variant” refers to an amino acid or peptide sequence having conservative amino acid substitutions, non-conservative amino acid substitutions (i.e. a degenerate variant), substitutions within the wobble position of each codon (i.e. DNA and RNA) encoding an amino acid, amino acids added to the C-terminus of a peptide, or a peptide having 60%, 70%, 80%, 90%, 95%, 96%, 97%, 98%, 99% sequence identity to a reference sequence.

The term “vector” refers to a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element).

### Switch Receptor

Disclosed herein is a chimeric (fusion) switch receptor having an extracellular domain of TIM-3, LAG-3, CD112R or DR5 and an intracellular domain of a pro-inflammatory interleukin and/or a co-stimulatory domain. For example, in some embodiments, the pro-inflammatory interleukin is selected from the group consisting of IL7Rα, IL2, IL15, IL21R, IL12Rb2 and IL6ST. In some embodiments, the co-stimulatory domain is selected from the group consisting of CD27, ICOS, OX40.

In some embodiments, the extracellular domain of TIM-3 has the amino acid sequence:

(SEQ ID NO: 65)
SEVEYRAEVGQNAYLPCFYTPAAPGNLVPVCWGKGACPVFECGNVVLRT
DERDVNYWTSRYWLNGDFRKGDVSLTIENVTLADSGIYCCRIQIPGIMN
DEKFNLKLVIKPAKVTPAPTLQRDFTAAFPRMLTTRGHGPAETQTLGSL
PDINLTQISTLANELRDSRLANDLRDSGATIRIG.

In some embodiments, the extracellular domain of LAG-3 has the amino acid sequence:

(SEQ ID NO: 66)
LQPGAEVPVVWAQEGAPAQLPCSPTIPLQDLSLLRRAGVTWQHQPDSGP
PAAAPGHPLAPGPHPAAPSSWGPRPRRYTVLSVGPGGLRSGRLPLQPRV
QLDERGRQRGDFSLWLRPARRADAGEYRAAVHLRDRALSCRLRLRLGQA
SMTASPPGSLRASDWVILNCSFSRPDRPASVHWFRNRGQGRVPVRESPH
HHLAESFLFLPQVSPMDSGPWGCILTYRDGFNVSIMYNLTVLGLEPPTP
LTVYAGAGSRVGLPCRLPAGVGTRSFLTAKWTPPGGGPDLLVTGDNGDF
TLRLEDVSQAQAGTYTCHIHLQEQQLNATVTLAIITVTPKSFGSPGSLG
KLLCEVTPVSGQERFVWSSLDTPSQRSFSGPWLEAQEAQLLSQPWQCQL
YQGERLLGAAVYFTELSSPGAQRSGRAPGALPAGHL.

In some embodiments, the extracellular domain of CD112R has the amino acid sequence:

(SEQ ID NO: 67)
TPEVWVQVRMEATELSSFTIRCGFLGSGSISLVTVSWGGPNGAGGTTLA
VLHPERGIRQWAPARQARWETQSSISLILEGSGASSPCANTTFCCKFAS
FPEGSWEACGSLPPSSDPGLSAPPTPAPILRADL.

In some embodiments, the extracellular domain of DR5 has the amino acid sequence:

(SEQ ID NO: 68)
ITQQDLAPQQRAAPQQKRSSPSEGLCPPGHHISEDGRDCISCKYGQDYS
THWNDLLFCLRCTRCDSGEVELSPCTTTRNTVCQCEEGTFREEDSPEMC
RKCRTGCPRGMVKVGDCTPWSDIECVHKESGTKHSGEVPAVEETVTSSP
GTPASPCS.

In some embodiments, the transmembrane and intracellular domain of IL7Rα has the amino acid sequence:

(SEQ ID NO: 69)
PILLTISILSFFSVALLVILACVLWKKRIKPIVWPSLPDHKKTLEHLCK
KPRKNLNVSFNPESFLDCQIHRVDDIQARDEVEGFLQDTFPQQLEESEK
QRLGGDVQSPNCPSEDVVITPESFGRDSSLTCLAGNVSACDAPILSSSR
SLDCRESGKNGPHVYQDLLLSLGTTNSTLPPPFSLQSGILTLNPVAQGQ
PILTSLGSNQEEAYVTMSSFYQNQ.

In some embodiments, the transmembrane and intracellular domain of the βcommon part of IL2R (IL2Rβ) and IL15 (IL2/IL15) has the amino acid sequence:

(SEQ ID NO: 70)
IPWLGHLLVGLSGAFGFIILVYLLINCRNTGPWLKKVLKCNTPDPSKFF
SQLSSEHGGDVQKWLSSPFPSSSFSPGGLAPEISPLEVLERDKVTQLLL
QQDKVPEPASLSSNHSLTSCFTNQGYFFFHLPDALEIEACQVYFTYDPY
SEEDPDEGVAGAPTGSSPQPLQPLSGEDDAYCTFPSRDDLLLFSPSLLG
GPSPPSTAPGGSGAGEERMPPSLQERVPRDWDPQPLGPPTPGVPDLVDF
QPPPELVLREAGEEVPDAGPREGVSFPWSRPPGQGEFRALNARLPLNTD
AYLSLQELQGQDPTHLV.

In some embodiments, the transmembrane and intracellular domain of IL21R has the amino acid sequence:

(SEQ ID NO: 71)
GWNPHLLLLLLLVIVFIPAFWSLKTHPLWRLWKKIWAVPSPERFFMPLY
KGCSGDFKKWVGAPFTGSSLELGPWSPEVPSTLEVYSCHPPRSPAKRLQ
LTELQEPAELVESDGVPKPSFWPTAQNSGGSAYSEERDRPYGLVSIDTV
TVLDAEGPCTWPCSCEDDGYPALDLDAGLEPSPGLEDPLLDAGTTVLSC
GCVSAGSPGLGGPLGSLLDRLKPPLADGEDWAGGLPWGGRSPGGVSESE
AGSPLAGLDMDTFDSGFVGSDCSSPVECDFTSPGDEGPPRSYLRQWVVI
PPPLSSPGPQAS.

In some embodiments, the transmembrane and intracellular domain of IL12Rb2 has the amino acid sequence:

(SEQ ID NO: 72)
WMAFVAPSICIAIIMVGIFSTHYFQQKVFVLLAALRPQWCSREIPDPAN
STCAKKYPIAEEKTQLPLDRLLIDWPTPEDPEPLVISEVLHQVTPVFRH
PPCSNWPQREKGIQGHQASEKDMMHSASSPPPPRALQAESRQLVDLYKV
LESRGSDPKPENPACPWTVLPAGDLPTHDGYLPSNIDDLPSHEAPLADS
LEELEPQHISLSVFPSSSLHPLTFSCGDKLTLDQLKMRCDSLML.

In some embodiments, the transmembrane and intracellular domain of IL6ST has the amino acid sequence:

(SEQ ID NO: 73)
AIVVPVCLAFLLTTLLGVLFCFNKRDLIKKHIWPNVPDPSKSHIAQWSP
HTPPRHNFNSKDQMYSDGNFTDVSVVEIEANDKKPFPEDLKSLDLFKKE
KINTEGHSSGIGGSSCMSSSRPSISSSDENESSQNTSSTVQYSTWHSGY
RHQVPSVQVFSRSESTQPLLDSEERPEDLQLVDHVDGGDGILPRQQYFK
QNCSQHESSPDISHFERSKQVSSVNEEDFVRLKQQISDHISQSCGSGQM
KMFQEVSAADAFGPGTEGQVERFETVGMEAATDEGMPKSYLPQTVRQGG
YMPQ.

Therefore, in some embodiments, the chimeric receptor is a Tim3-IL7Rα fusion and has the amino acid sequence:

(SEQ ID NO: 1)
SEVEYRAEVGQNAYLPCFYTPAAPGNLVPVCWGKGACPVFECGNVVLRT
DERDVNYWTSRYWLNGDFRKGDVSLTIENVTLADSGIYCCRIQIPGIMN
DEKFNLKLVIKPAKVTPAPTLQRDFTAAFPRMLTTRGHGPAETQTLGSL
PDINLTQISTLANELRDSRLANDLRDSGATIRIGPILLTISILSFFSVA
LLVILACVLWKKRIKPIVWPSLPDHKKTLEHLCKKPRKNLNVSFNPESF
LDCQIHRVDDIQARDEVEGFLQDTFPQQLEESEKQRLGGDVQSPNCPSE
DVVITPESFGRDSSLTCLAGNVSACDAPILSSSRSLDCRESGKNGPHVY
QDLLLSLGTTNSTLPPPFSLQSGILTLNPVAQGQPILTSLGSNQEEAYV
TMSSFYQNQ

Therefore, in some embodiments, the chimeric receptor is encoded by the nucleic acid sequence:

(SEQ ID NO: 33)
TCAGAAGTGGAATACAGAGCGGAGGTCGGTCAGAATGCCTATCTGCCCT
GCTTCTACACCCCAGCCGCCCCAGGGAACCTCGTGCCCGTCTGCTGGGG
CAAAGGAGCCTGTCCTGTGTTTGAATGTGGCAACGTGGTGCTCAGGACT
GATGAAAGGGATGTGAATTATTGGACATCCAGATACTGGCTAAATGGGG
ATTTCCGCAAAGGAGATGTGTCCCTGACCATAGAGAATGTGACTCTAGC
AGACAGTGGGATCTACTGCTGCCGGATCCAAATCCCAGGCATAATGAAT
GATGAAAAATTTAACCTGAAGTTGGTCATCAAACCAGCCAAGGTCACCC
CTGCACCGACTCTGCAGAGAGACTTCACTGCAGCCTTTCCAAGGATGCT
TACCACCAGGGGACATGGCCCAGCAGAGACACAGACACTGGGGAGCCTC
CCTGATATAAATCTAACACAAATATCCACATTGGCCAATGAGTTACGGG
ACTCTAGATTGGCCAATGACTTACGGGACTCTGGAGCAACCATCAGAAT
AGGCCCTATCTTACTAACCATCAGCATTTTGAGTTTTTTCTCTGTCGCT
CTGTTGGTCATCTTGGCCTGTGTGTTATGGAAAAAAAGGATTAAGCCTA
TCGTATGGCCCAGTCTCCCCGATCATAAGAAGACTCTGGAACATCTTTG
TAAGAAACCAAGAAAAAATTTAAATGTGAGTTTCAATCCTGAAAGTTTC
CTGGACTGCCAGATTCATAGGGTGGATGACATTCAAGCTAGAGATGAAG
TGGAAGGTTTTCTGCAAGATACGTTTCCTCAGCAACTAGAAGAATCTGA
GAAGCAGAGGCTTGGAGGGGATGTGCAGAGCCCCAACTGCCCATCTGAG
GATGTAGTCGTCACTCCAGAAAGCTTTGGAAGAGATTCATCCCTCACAT
GCCTGGCTGGGAATGTCAGTGCATGTGACGCCCCTATTCTCTCCTCTTC
CAGGTCCCTAGACTGCAGGGAGAGTGGCAAGAATGGGCCTCATGTGTAC
CAGGACCTCCTGCTTAGCCTTGGGACTACAAACAGCACGCTGCCCCCTC
CATTTTCTCTCCAATCTGGAATCCTGACATTGAACCCAGTTGCTCAGGG
TCAGCCCATTCTTACTTCCCTGGGATCAAATCAAGAAGAAGCATATGTC
ACCATGTCCAGCTTCTACCAAAACCAGTGA.

Therefore, in some embodiments, the chimeric receptor is a Tim3/IL2Rβ fusion and has the amino acid sequence:

(SEQ ID NO: 2)
SEVEYRAEVGQNAYLPCFYTPAAPGNLVPVCWGKGACPVFECGNVVLRT
DERDVNYWTSRYWLNGDFRKGDVSLTIENVTLADSGIYCCRIQIPGIMN
DEKFNLKLVIKPAKVTPAPTLQRDFTAAFPRMLTTRGHGPAETQTLGSL
PDINLTQISTLANELRDSRLANDLRDSGATIRIGIPWLGHLLVGLSGAF
GFIILVYLLINCRNTGPWLKKVLKCNTPDPSKFFSQLSSEHGGDVQKWL
SSPFPSSSFSPGGLAPEISPLEVLERDKVTQLLLQQDKVPEPASLSSNH
SLTSCFTNQGYFFFHLPDALEIEACQVYFTYDPYSEEDPDEGVAGAPTG
SSPQPLQPLSGEDDAYCTFPSRDDLLLFSPSLLGGPSPPSTAPGGSGAG
EERMPPSLQERVPRDWDPQPLGPPTPGVPDLVDFQPPPELVLREAGEEV
PDAGPREGVSFPWSRPPGQGEFRALNARLPLNTDAYLSLQELQGQDPTH
LV.

Therefore, in some embodiments, the chimeric receptor is encoded by the

(SEQ ID NO: 34)
TCAGAAGTGGAATACAGAGCGGAGGTCGGTCAGAATGCCTATCTGCCCT
GCTTCTACACCCCAGCCGCCCCAGGGAACCTCGTGCCCGTCTGCTGGGG
CAAAGGAGCCTGTCCTGTGTTTGAATGTGGCAACGTGGTGCTCAGGACT
GATGAAAGGGATGTGAATTATTGGACATCCAGATACTGGCTAAATGGGG
ATTTCCGCAAAGGAGATGTGTCCCTGACCATAGAGAATGTGACTCTAGC
AGACAGTGGGATCTACTGCTGCCGGATCCAAATCCCAGGCATAATGAAT
GATGAAAAATTTAACCTGAAGTTGGTCATCAAACCAGCCAAGGTCACCC
CTGCACCGACTCTGCAGAGAGACTTCACTGCAGCCTTTCCAAGGATGCT
TACCACCAGGGGACATGGCCCAGCAGAGACACAGACACTGGGGAGCCTC
CCTGATATAAATCTAACACAAATATCCACATTGGCCAATGAGTTACGGG
ACTCTAGATTGGCCAATGACTTACGGGACTCTGGAGCAACCATCAGAAT
AGGCATTCCGTGGCTCGGCCACCTCCTCGTGGGCCTCAGCGGGGCTTTT
GGCTTCATCATCTTAGTGTACTTGCTGATCAACTGCAGGAACACCGGGC
CATGGCTGAAGAAGGTCCTGAAGTGTAACACCCCAGACCCCTCGAAGTT
CTTTTCCCAGCTGAGCTCAGAGCATGGAGGAGACGTCCAGAAGTGGCTC
TCTTCGCCCTTCCCCTCATCGTCCTTCAGCCCTGGCGGCCTGGCACCTG
AGATCTCGCCACTAGAAGTGCTGGAGAGGGACAAGGTGACGCAGCTGCT
CCTGCAGCAGGACAAGGTGCCTGAGCCCGCATCCTTAAGCAGCAACCAC
TCGCTGACCAGCTGCTTCACCAACCAGGGTTACTTCTTCTTCCACCTCC
CGGATGCCTTGGAGATAGAGGCCTGCCAGGTGTACTTTACTTACGACCC
CTACTCAGAGGAAGACCCTGATGAGGGTGTGGCCGGGGCACCCACAGGG
TCTTCCCCCCAACCCCTGCAGCCTCTGTCAGGGGAGGACGACGCCTACT
GCACCTTCCCCTCCAGGGATGACCTGCTGCTCTTCTCCCCCAGTCTCCT
CGGTGGCCCCAGCCCCCCAAGCACTGCCCCTGGGGGCAGTGGGGCCGGT
GAAGAGAGGATGCCCCCTTCTTTGCAAGAAAGAGTCCCCAGAGACTGGG
ACCCCCAGCCCCTGGGGCCTCCCACCCCAGGAGTCCCAGACCTGGTGGA
TTTTCAGCCACCCCCTGAGCTGGTGCTGCGAGAGGCTGGGGAGGAGGTC
CCTGACGCTGGCCCCAGGGAGGGAGTCAGTTTCCCCTGGTCCAGGCCTC
CTGGGCAGGGGGAGTTCAGGGCCCTTAATGCTCGCCTGCCCCTGAACAC
TGATGCCTACTTGTCCCTCCAAGAACTCCAGGGTCAGGACCCAACTCAC
TTGGTGTAG.

Therefore, in some embodiments, the chimeric receptor is a Tim3-IL21R fusion and has the amino acid sequence:

(SEQ ID NO: 3)
SEVEYRAEVGQNAYLPCFYTPAAPGNLVPVCWGKGACPVFECGNVVLRT
DERDVNYWTSRYWLNGDFRKGDVSLTIENVTLADSGIYCCRIQIPGIMN
DEKFNLKLVIKPAKVTPAPTLQRDFTAAFPRMLTTRGHGPAETQTLGSL
PDINLTQISTLANELRDSRLANDLRDSGATIRIGGWNPHLLLLLLLVIV
FIPAFWSLKTHPLWRLWKKIWAVPSPERFFMPLYKGCSGDFKKWVGAPF
TGSSLELGPWSPEVPSTLEVYSCHPPRSPAKRLQLTELQEPAELVESDG
VPKPSFWPTAQNSGGSAYSEERDRPYGLVSIDTVTVLDAEGPCTWPCSC
EDDGYPALDLDAGLEPSPGLEDPLLDAGTTVLSCGCVSAGSPGLGGPLG
SLLDRLKPPLADGEDWAGGLPWGGRSPGGVSESEAGSPLAGLDMDTFDS
GFVGSDCSSPVECDFTSPGDEGPPRSYLRQWVVIPPPLSSPGPQAS.

Therefore, in some embodiments, the chimeric receptor is encoded by the

(SEQ ID NO: 35)
TCAGAAGTGGAATACAGAGCGGAGGTCGGTCAGAATGCCTATCTGCCCT
GCTTCTACACCCCAGCCGCCCCAGGGAACCTCGTGCCCGTCTGCTGGGG
CAAAGGAGCCTGTCCTGTGTTTGAATGTGGCAACGTGGTGCTCAGGACT
GATGAAAGGGATGTGAATTATTGGACATCCAGATACTGGCTAAATGGGG
ATTTCCGCAAAGGAGATGTGTCCCTGACCATAGAGAATGTGACTCTAGC
AGACAGTGGGATCTACTGCTGCCGGATCCAAATCCCAGGCATAATGAAT
GATGAAAAATTTAACCTGAAGTTGGTCATCAAACCAGCCAAGGTCACCC
CTGCACCGACTCTGCAGAGAGACTTCACTGCAGCCTTTCCAAGGATGCT
TACCACCAGGGGACATGGCCCAGCAGAGACACAGACACTGGGGAGCCTC
CCTGATATAAATCTAACACAAATATCCACATTGGCCAATGAGTTACGGG
ACTCTAGATTGGCCAATGACTTACGGGACTCTGGAGCAACCATCAGAAT
AGGCGGCTGGAACCCTCACCTGCTGCTTCTCCTCCTGCTTGTCATAGTC
TTCATTCCTGCCTTCTGGAGCCTGAAGACCCATCCATTGTGGAGGCTAT
GGAAGAAGATATGGGCCGTCCCCAGCCCTGAGCGGTTCTTCATGCCCCT
GTACAAGGGCTGCAGCGGAGACTTCAAGAAATGGGTGGGTGCACCCTTC
ACTGGCTCCAGCCTGGAGCTGGGACCCTGGAGCCCAGAGGTGCCCTCCA
CCCTGGAGGTGTACAGCTGCCACCCACCACGGAGCCCGGCCAAGAGGCT
GCAGCTCACGGAGCTACAAGAACCAGCAGAGCTGGTGGAGTCTGACGGT
GTGCCCAAGCCCAGCTTCTGGCCGACAGCCCAGAACTCGGGGGGCTCAG
CTTACAGTGAGGAGAGGGATCGGCCATACGGCCTGGTGTCCATTGACAC
AGTGACTGTGCTAGATGCAGAGGGGCCATGCACCTGGCCCTGCAGCTGT
GAGGATGACGGCTACCCAGCCCTGGACCTGGATGCTGGCCTGGAGCCCA
GCCCAGGCCTAGAGGACCCACTCTTGGATGCAGGGACCACAGTCCTGTC
CTGTGGCTGTGTCTCAGCTGGCAGCCCTGGGCTAGGAGGGCCCCTGGGA
AGCCTCCTGGACAGACTAAAGCCACCCCTTGCAGATGGGGAGGACTGGG
CTGGGGGACTGCCCTGGGGTGGCCGGTCACCTGGAGGGGTCTCAGAGAG
TGAGGCGGGCTCACCCCTGGCCGGCCTGGATATGGACACGTTTGACAGT
GGCTTTGTGGGCTCTGACTGCAGCAGCCCTGTGGAGTGTGACTTCACCA
GCCCCGGGGACGAAGGACCCCCCCGGAGCTACCTCCGCCAGTGGGTGGT
CATTCCTCCGCCACTTTCGAGCCCTGGACCCCAGGCCAGCTAATGA.

Therefore, in some embodiments, the chimeric receptor is a Tim3-IL12Rb2 fusion and has the amino acid sequence:

(SEQ ID NO: 4)
SEVEYRAEVGQNAYLPCFYTPAAPGNLVPVCWGKGACPVFECGNVVLRT
DERDVNYWTSRYWLNGDFRKGDVSLTIENVTLADSGIYCCRIQIPGIMN
DEKFNLKLVIKPAKVTPAPTLQRDFTAAFPRMLTTRGHGPAETQTLGSL
PDINLTQISTLANELRDSRLANDLRDSGATIRIGWMAFVAPSICIAIIM
VGIFSTHYFQQKVFVLLAALRPQWCSREIPDPANSTCAKKYPIAEEKTQ
LPLDRLLIDWPTPEDPEPLVISEVLHQVTPVFRHPPCSNWPQREKGIQG
HQASEKDMMHSASSPPPPRALQAESRQLVDLYKVLESRGSDPKPENPAC
PWTVLPAGDLPTHDGYLPSNIDDLPSHEAPLADSLEELEPQHISLSVFP
SSSLHPLTFSCGDKLTLDQLKMRCDSLML.

Therefore, in some embodiments, the chimeric receptor is encoded by the

(SEQ ID NO: 36)
TCAGAAGTGGAATACAGAGCGGAGGTCGGTCAGAATGCCTATCTGCCCT
GCTTCTACACCCCAGCCGCCCCAGGGAACCTCGTGCCCGTCTGCTGGGG
CAAAGGAGCCTGTCCTGTGTTTGAATGTGGCAACGTGGTGCTCAGGACT
GATGAAAGGGATGTGAATTATTGGACATCCAGATACTGGCTAAATGGGG
ATTTCCGCAAAGGAGATGTGTCCCTGACCATAGAGAATGTGACTCTAGC
AGACAGTGGGATCTACTGCTGCCGGATCCAAATCCCAGGCATAATGAAT
GATGAAAAATTTAACCTGAAGTTGGTCATCAAACCAGCCAAGGTCACCC
CTGCACCGACTCTGCAGAGAGACTTCACTGCAGCCTTTCCAAGGATGCT
TACCACCAGGGGACATGGCCCAGCAGAGACACAGACACTGGGGAGCCTC
CCTGATATAAATCTAACACAAATATCCACATTGGCCAATGAGTTACGGG
ACTCTAGATTGGCCAATGACTTACGGGACTCTGGAGCAACCATCAGAAT
AGGCTGGATGGCGTTTGTGGCACCAAGCATTTGCATTGCTATCATCATG
GTGGGCATTTTCTCAACGCATTACTTCCAGCAAAAGGTGTTTGTTCTCC
TAGCAGCCCTCAGACCTCAGTGGTGTAGCAGAGAAATTCCAGATCCAGC
AAATAGCACTTGCGCTAAGAAATATCCCATTGCAGAGGAGAAGACACAG
CTGCCCTTGGACAGGCTCCTGATAGACTGGCCCACGCCTGAAGATCCTG
AACCGCTGGTCATCAGTGAAGTCCTTCATCAAGTGACCCCAGTTTTCAG
ACATCCCCCCTGCTCCAACTGGCCACAAAGGGAAAAAGGAATCCAAGGT
CATCAGGCCTCTGAGAAAGACATGATGCACAGTGCCTCAAGCCCACCAC
CTCCAAGAGCTCTCCAAGCTGAGAGCAGACAACTGGTGGATCTGTACAA
GGTGCTGGAGAGCAGGGGCTCCGACCCAAAGCCAGAAAACCCAGCCTGT
CCCTGGACGGTGCTCCCAGCAGGTGACCTTCCCACCCATGATGGCTACT
TACCCTCCAACATAGATGACCTCCCCTCACATGAGGCACCTCTCGCTGA
CTCTCTGGAAGAACTGGAGCCTCAGCACATCTCCCTTTCTGTTTTCCCC
TCAAGTTCTCTTCACCCACTCACCTTCTCCTGTGGTGATAAGCTGACTC
TGGATCAGTTAAAGATGAGGTGTGACTCCCTCATGCTCTGA.

Therefore, in some embodiments, the chimeric receptor is a Tim3-IL6ST fusion and has the amino acid sequence:

(SEQ ID NO: 5)
SEVEYRAEVGQNAYLPCFYTPAAPGNLVPVCWGKGACPVFECGNVVLRT
DERDVNYWTSRYWLNGDFRKGDVSLTIENVTLADSGIYCCRIQIPGIMN
DEKFNLKLVIKPAKVTPAPTLQRDFTAAFPRMLTTRGHGPAETQTLGSL
PDINLTQISTLANELRDSRLANDLRDSGATIRIGAIVVPVCLAFLLTTL
LGVLFCFNKRDLIKKHIWPNVPDPSKSHIAQWSPHTPPRHNFNSKDQMY
SDGNFTDVSVVEIEANDKKPFPEDLKSLDLFKKEKINTEGHSSGIGGSS
CMSSSRPSISSSDENESSQNTSSTVQYSTVVHSGYRHQVPSVQVFSRSE
STQPLLDSEERPEDLQLVDHVDGGDGILPRQQYFKQNCSQHESSPDISH
FERSKQVSSVNEEDFVRLKQQISDHISQSCGSGQMKMFQEVSAADAFGP
GTEGQVERFETVGMEAATDEGMPKSYLPQTVRQGGYMPQ.

Therefore, in some embodiments, the chimeric receptor is encoded by the

(SEQ ID NO: 37)
TCAGAAGTGGAATACAGAGCGGAGGTCGGTCAGAATGCCTATCTGCCCT
GCTTCTACACCCCAGCCGCCCCAGGGAACCTCGTGCCCGTCTGCTGGGG
CAAAGGAGCCTGTCCTGTGTTTGAATGTGGCAACGTGGTGCTCAGGACT
GATGAAAGGGATGTGAATTATTGGACATCCAGATACTGGCTAAATGGGG
ATTTCCGCAAAGGAGATGTGTCCCTGACCATAGAGAATGTGACTCTAGC
AGACAGTGGGATCTACTGCTGCCGGATCCAAATCCCAGGCATAATGAAT
GATGAAAAATTTAACCTGAAGTTGGTCATCAAACCAGCCAAGGTCACCC
CTGCACCGACTCTGCAGAGAGACTTCACTGCAGCCTTTCCAAGGATGCT
TACCACCAGGGGACATGGCCCAGCAGAGACACAGACACTGGGGAGCCTC
CCTGATATAAATCTAACACAAATATCCACATTGGCCAATGAGTTACGGG
ACTCTAGATTGGCCAATGACTTACGGGACTCTGGAGCAACCATCAGAAT
AGGCGCCATAGTCGTGCCTGTTTGCTTAGCATTCCTATTGACAACTCTT
CTGGGAGTGCTGTTCTGCTTTAATAAGCGAGACCTAATTAAAAAACACA
TCTGGCCTAATGTTCCAGATCCTTCAAAGAGTCATATTGCCCAGTGGTC
ACCTCACACTCCTCCAAGGCACAATTTTAATTCAAAAGATCAAATGTAT
TCAGATGGCAATTTCACTGATGTAAGTGTTGTGGAAATAGAAGCAAATG
ACAAAAAGCCTTTTCCAGAAGATCTGAAATCATTGGACCTGTTCAAAAA
GGAAAAAATTAATACTGAAGGACACAGCAGTGGTATTGGGGGGTCTTCA
TGCATGTCATCTTCTAGGCCAAGCATTTCTAGCAGTGATGAAAATGAAT
CTTCACAAAACACTTCGAGCACTGTCCAGTATTCTACCGTGGTACACAG
TGGCTACAGACACCAAGTTCCGTCAGTCCAAGTCTTCTCAAGATCCGAG
TCTACCCAGCCCTTGTTAGATTCAGAGGAGCGGCCAGAAGATCTACAAT
TAGTAGATCATGTAGATGGCGGTGATGGTATTTTGCCCAGGCAACAGTA
CTTCAAACAGAACTGCAGTCAGCATGAATCCAGTCCAGATATTTCACAT
TTTGAAAGGTCAAAGCAAGTTTCATCAGTCAATGAGGAAGATTTTGTTA
GACTTAAACAGCAGATTTCAGATCATATTTCACAATCCTGTGGATCTGG
GCAAATGAAAATGTTTCAGGAAGTTTCTGCAGCAGATGCTTTTGGTCCA
GGTACTGAGGGACAAGTAGAAAGATTTGAAACAGTTGGCATGGAGGCTG
CGACTGATGAAGGCATGCCTAAAAGTTACTTACCACAGACTGTACGGCA
AGGCGGCTACATGCCTCAGT.

Therefore, in some embodiments, the chimeric receptor is a Tim3-CD27 fusion and has the amino acid sequence:

(SEQ ID NO: 6)
SEVEYRAEVGQNAYLPCFYTPAAPGNLVPVCWGKGACPVFECGNVVLRT
DERDVNYWTSRYWLNGDFRKGDVSLTIENVTLADSGIYCCRIQIPGIMN
DEKFNLKLVIKPAKVTPAPTLQRDFTAAFPRMLTTRGHGPAETQTLGSL
PDINLTQISTLANELRDSRLANDLRDSGATIRIGILVIFSGMFLVFTLA
GALFLHQRRKYRSNKGESPVEPAEPCRYSCPREEEGSTIPIQEDYRKPE
PACSP.

Therefore, in some embodiments, the chimeric receptor is encoded by the

(SEQ ID NO: 38)
TCAGAAGTGGAATACAGAGCGGAGGTCGGTCAGAATGCCTATCTGCCCT
GCTTCTACACCCCAGCCGCCCCAGGGAACCTCGTGCCCGTCTGCTGGGG
CAAAGGAGCCTGTCCTGTGTTTGAATGTGGCAACGTGGTGCTCAGGACT
GATGAAAGGGATGTGAATTATTGGACATCCAGATACTGGCTAAATGGGG
ATTTCCGCAAAGGAGATGTGTCCCTGACCATAGAGAATGTGACTCTAGC
AGACAGTGGGATCTACTGCTGCCGGATCCAAATCCCAGGCATAATGAAT
GATGAAAAATTTAACCTGAAGTTGGTCATCAAACCAGCCAAGGTCACCC
CTGCACCGACTCTGCAGAGAGACTTCACTGCAGCCTTTCCAAGGATGCT
TACCACCAGGGGACATGGCCCAGCAGAGACACAGACACTGGGGAGCCTC
CCTGATATAAATCTAACACAAATATCCACATTGGCCAATGAGTTACGGG
ACTCTAGATTGGCCAATGACTTACGGGACTCTGGAGCAACCATCAGAAT
AGGCATCCTTGTGATCTTCTCTGGAATGTTCCTTGTTTTCACCCTGGCC
GGGGCCCTGTTCCTCCATCAACGAAGGAAATATAGATCAAACAAAGGAG
AAAGTCCTGTGGAGCCTGCAGAGCCTTGTCGTTACAGCTGCCCCAGGGA
GGAGGAGGGCAGCACCATCCCCATCCAGGAGGATTACCGAAAACCGGAG
CCTGCCTGCTCCCCCTGA.

Therefore, in some embodiments, the chimeric receptor is a Tim3-ICOS fusion and has the amino acid sequence:

(SEQ ID NO: 7)
SEVEYRAEVGQNAYLPCFYTPAAPGNLVPVCWGKGACPVFECGNVVLRT
DERDVNYWTSRYWLNGDFRKGDVSLTIENVTLADSGIYCCRIQIPGIMN
DEKFNLKLVIKPAKVTPAPTLQRDFTAAFPRMLTTRGHGPAETQTLGSL
PDINLTQISTLANELRDSRLANDLRDSGATIRIGFWLPIGCAAFVVVCI
LGCILICWLTKKKYSSSVHDPNGEYMFMRAVNTAKKSRLTDVTL.

Therefore, in some embodiments, the chimeric receptor is encoded by the nucleic acid sequence:

(SEQ ID NO: 39)
TCAGAAGTGGAATACAGAGCGGAGGTCGGTCAGAATGCCTATCTGCCCT
GCTTCTACACCCCAGCCGCCCCAGGGAACCTCGTGCCCGTCTGCTGGGG
CAAAGGAGCCTGTCCTGTGTTTGAATGTGGCAACGTGGTGCTCAGGACT
GATGAAAGGGATGTGAATTATTGGACATCCAGATACTGGCTAAATGGGG
ATTTCCGCAAAGGAGATGTGTCCCTGACCATAGAGAATGTGACTCTAGC
AGACAGTGGGATCTACTGCTGCCGGATCCAAATCCCAGGCATAATGAAT
GATGAAAAATTTAACCTGAAGTTGGTCATCAAACCAGCCAAGGTCACCC
CTGCACCGACTCTGCAGAGAGACTTCACTGCAGCCTTTCCAAGGATGCT
TACCACCAGGGGACATGGCCCAGCAGAGACACAGACACTGGGGAGCCTC
CCTGATATAAATCTAACACAAATATCCACATTGGCCAATGAGTTACGGG
ACTCTAGATTGGCCAATGACTTACGGGACTCTGGAGCAACCATCAGAAT
AGGCTTCTGGTTACCCATAGGATGTGCAGCCTTTGTTGTAGTCTGCATT
TTGGGATGCATACTTATTTGTTGGCTTACAAAAAAGAAGTATTCATCCA
GTGTGCACGACCCTAACGGTGAATACATGTTCATGAGAGCAGTGAACAC
AGCCAAAAAATCTAGACTCACAGATGTGACCCTATAA.

Therefore, in some embodiments, the chimeric receptor is a Tim3-OX40 fusion and has the amino acid sequence:

(SEQ ID NO: 8)
SEVEYRAEVGQNAYLPCFYTPAAPGNLVPVCWGKGACPVFECGNVVLRT
DERDVNYWTSRYWLNGDFRKGDVSLTIENVTLADSGIYCCRIQIPGIMN
DEKFNLKLVIKPAKVTPAPTLQRDFTAAFPRMLTTRGHGPAETQTLGSL
PDINLTQISTLANELRDSRLANDLRDSGATIRIGVAAILGLGLVLGLLG
PLAILLALYLLRRDQRLPPDAHKPPGGGSFRTPIQEEQADAHSTLAKI.

Therefore, in some embodiments, the chimeric receptor is encoded by the

(SEQ ID NO: 40)
TCAGAAGTGGAATACAGAGCGGAGGTCGGTCAGAATGCCTATCTGCCCT
GCTTCTACACCCCAGCCGCCCCAGGGAACCTCGTGCCCGTCTGCTGGGG
CAAAGGAGCCTGTCCTGTGTTTGAATGTGGCAACGTGGTGCTCAGGACT
GATGAAAGGGATGTGAATTATTGGACATCCAGATACTGGCTAAATGGGG
ATTTCCGCAAAGGAGATGTGTCCCTGACCATAGAGAATGTGACTCTAGC
AGACAGTGGGATCTACTGCTGCCGGATCCAAATCCCAGGCATAATGAAT
GATGAAAAATTTAACCTGAAGTTGGTCATCAAACCAGCCAAGGTCACCC
CTGCACCGACTCTGCAGAGAGACTTCACTGCAGCCTTTCCAAGGATGCT
TACCACCAGGGGACATGGCCCAGCAGAGACACAGACACTGGGGAGCCTC
CCTGATATAAATCTAACACAAATATCCACATTGGCCAATGAGTTACGGG
ACTCTAGATTGGCCAATGACTTACGGGACTCTGGAGCAACCATCAGAAT
AGGCGTTGCCGCCATCCTGGGCCTGGGCCTGGTGCTGGGGCTGCTGGGC
CCCCTGGCCATCCTGCTGGCCCTGTACCTGCTCCGGAGGGACCAGAGGC
TGCCCCCCGATGCCCACAAGCCCCCTGGGGGAGGCAGTTTCCGGACCCC
CATCCAAGAGGAGCAGGCCGACGCCCACTCCACCCTGGCCAAGATCTG
A.

Therefore, in some embodiments, the chimeric receptor is a LAG3-IL7Rα fusion and has the amino acid sequence:

(SEQ ID NO: 9)
LQPGAEVPVVWAQEGAPAQLPCSPTIPLQDLSLLRRAGVTWQHQPDSGP
PAAAPGHPLAPGPHPAAPSSWGPRPRRYTVLSVGPGGLRSGRLPLQPRV
QLDERGRQRGDFSLWLRPARRADAGEYRAAVHLRDRALSCRLRLRLGQA
SMTASPPGSLRASDWVILNCSFSRPDRPASVHWFRNRGQGRVPVRESPH
HHLAESFLFLPQVSPMDSGPWGCILTYRDGFNVSIMYNLTVLGLEPPTP
LTVYAGAGSRVGLPCRLPAGVGTRSFLTAKWTPPGGGPDLLVTGDNGDF
TLRLEDVSQAQAGTYTCHIHLQEQQLNATVTLAIITVTPKSFGSPGSLG
KLLCEVTPVSGQERFVWSSLDTPSQRSFSGPWLEAQEAQLLSQPWQCQL
YQGERLLGAAVYFTELSSPGAQRSGRAPGALPAGHLPILLTISILSFFS
VALLVILACVLWKKRIKPIVWPSLPDHKKTLEHLCKKPRKNLNVSFNPE
SFLDCQIHRVDDIQARDEVEGFLQDTFPQQLEESEKQRLGGDVQSPNCP
SEDVVITPESFGRDSSLTCLAGNVSACDAPILSSSRSLDCRESGKNGPH
VYQDLLLSLGTTNSTLPPPFSLQSGILTLNPVAQGQPILTSLGSNQEEA
YVTMSSFYQNQ.

Therefore, in some embodiments, the chimeric receptor is encoded by the

(SEQ ID NO: 41)
CTCCAGCCAGGGGCTGAGGTCCCGGTGGTGTGGGCCCAGGAGGGGGCTC
CTGCCCAGCTCCCCTGCAGCCCCACAATCCCCCTCCAGGATCTCAGCCT
TCTGCGAAGAGCAGGGGTCACTTGGCAGCATCAGCCAGACAGTGGCCCG
CCCGCTGCCGCCCCCGGCCATCCCCTGGCCCCCGGCCCTCACCCGGCGG
CGCCCTCCTCCTGGGGGCCCAGGCCCCGCCGCTACACGGTGCTGAGCGT
GGGTCCCGGAGGCCTGCGCAGCGGGAGGCTGCCCCTGCAGCCCCGCGTC
CAGCTGGATGAGCGCGGCCGGCAGCGCGGGGACTTCTCGCTATGGCTGC
GCCCAGCCCGGCGCGCGGACGCCGGCGAGTACCGCGCCGCGGTGCACCT
CAGGGACCGCGCCCTCTCCTGCCGCCTCCGTCTGCGCCTGGGCCAGGCC
TCGATGACTGCCAGCCCCCCAGGATCTCTCAGAGCCTCCGACTGGGTCA
TTTTGAACTGCTCCTTCAGCCGCCCTGACCGCCCAGCCTCTGTGCATTG
GTTCCGGAACCGGGGCCAGGGCCGAGTCCCTGTCCGGGAGTCCCCCCAT
CACCACTTAGCGGAAAGCTTCCTCTTCCTGCCCCAAGTCAGCCCCATGG
ACTCTGGGCCCTGGGGCTGCATCCTCACCTACAGAGATGGCTTCAACGT
CTCCATCATGTATAACCTCACTGTTCTGGGTCTGGAGCCCCCAACTCCC
TTGACAGTGTACGCTGGAGCAGGTTCCAGGGTGGGGCTGCCCTGCCGCC
TGCCTGCTGGTGTGGGGACCCGGTCTTTCCTCACTGCCAAGTGGACTCC
TCCTGGGGGAGGCCCTGACCTCCTGGTGACTGGAGACAATGGCGACTTT
ACCCTTCGACTAGAGGATGTGAGCCAGGCCCAGGCTGGGACCTACACCT
GCCATATCCATCTGCAGGAACAGCAGCTCAATGCCACTGTCACATTGGC
AATCATCACAGTGACTCCCAAATCCTTTGGGTCACCTGGATCCCTGGGG
AAGCTGCTTTGTGAGGTGACTCCAGTATCTGGACAAGAACGCTTTGTGT
GGAGCTCTCTGGACACCCCATCCCAGAGGAGTTTCTCAGGACCTTGGCT
GGAGGCACAGGAGGCCCAGCTCCTTTCCCAGCCTTGGCAATGCCAGCTG
TACCAGGGGGAGAGGCTTCTTGGAGCAGCAGTGTACTTCACAGAGCTGT
CTAGCCCAGGTGCCCAACGCTCTGGGAGAGCCCCAGGTGCCCTCCCAGC
AGGCCACCTCCCTATCTTACTAACCATCAGCATTTTGAGTTTTTTCTCT
GTCGCTCTGTTGGTCATCTTGGCCTGTGTGTTATGGAAAAAAAGGATTA
AGCCTATCGTATGGCCCAGTCTCCCCGATCATAAGAAGACTCTGGAACA
TCTTTGTAAGAAACCAAGAAAAAATTTAAATGTGAGTTTCAATCCTGAA
AGTTTCCTGGACTGCCAGATTCATAGGGTGGATGACATTCAAGCTAGAG
ATGAAGTGGAAGGTTTTCTGCAAGATACGTTTCCTCAGCAACTAGAAGA
ATCTGAGAAGCAGAGGCTTGGAGGGGATGTGCAGAGCCCCAACTGCCCA
TCTGAGGATGTAGTCGTCACTCCAGAAAGCTTTGGAAGAGATTCATCCC
TCACATGCCTGGCTGGGAATGTCAGTGCATGTGACGCCCCTATTCTCTC
CTCTTCCAGGTCCCTAGACTGCAGGGAGAGTGGCAAGAATGGGCCTCAT
GTGTACCAGGACCTCCTGCTTAGCCTTGGGACTACAAACAGCACGCTGC
CCCCTCCATTTTCTCTCCAATCTGGAATCCTGACATTGAACCCAGTTGC
TCAGGGTCAGCCCATTCTTACTTCCCTGGGATCAAATCAAGAAGAAGCA
TATGTCACCATGTCCAGCTTCTACCAAAACCAGTGA.

Therefore, in some embodiments, the chimeric receptor is a LAG3-β/IL2, 15 fusion and has the amino acid sequence:

(SEQ ID NO: 10)
LQPGAEVPVVWAQEGAPAQLPCSPTIPLQDLSLLRRAGVTWQHQPDSGP
PAAAPGHPLAPGPHPAAPSSWGPRPRRYTVLSVGPGGLRSGRLPLQPRV
QLDERGRQRGDFSLWLRPARRADAGEYRAAVHLRDRALSCRLRLRLGQA
SMTASPPGSLRASDWVILNCSFSRPDRPASVHWFRNRGQGRVPVRESPH
HHLAESFLFLPQVSPMDSGPWGCILTYRDGFNVSIMYNLTVLGLEPPTP
LTVYAGAGSRVGLPCRLPAGVGTRSFLTAKWTPPGGGPDLLVTGDNGDF
TLRLEDVSQAQAGTYTCHIHLQEQQLNATVTLAIITVTPKSFGSPGSLG
KLLCEVTPVSGQERFVWSSLDTPSQRSFSGPWLEAQEAQLLSQPWQCQL
YQGERLLGAAVYFTELSSPGAQRSGRAPGALPAGHLIPWLGHLLVGLSG
AFGFIILVYLLINCRNTGPWLKKVLKCNTPDPSKFFSQLSSEHGGDVQK
WLSSPFPSSSFSPGGLAPEISPLEVLERDKVTQLLLQQDKVPEPASLSS
NHSLTSCFTNQGYFFFHLPDALEIEACQVYFTYDPYSEEDPDEGVAGAP
TGSSPQPLQPLSGEDDAYCTFPSRDDLLLFSPSLLGGPSPPSTAPGGSG
AGEERMPPSLQERVPRDWDPQPLGPPTPGVPDLVDFQPPPELVLREAGE
EVPDAGPREGVSFPWSRPPGQGEFRALNARLPLNTDAYLSLQELQGQDP
THLV.

Therefore, in some embodiments, the chimeric receptor is encoded by the

(SEQ ID NO: 42)
CTCCAGCCAGGGGCTGAGGTCCCGGTGGTGTGGGCCCAGGAGGGGGCTC
CTGCCCAGCTCCCCTGCAGCCCCACAATCCCCCTCCAGGATCTCAGCCT
TCTGCGAAGAGCAGGGGTCACTTGGCAGCATCAGCCAGACAGTGGCCCG
CCCGCTGCCGCCCCCGGCCATCCCCTGGCCCCCGGCCCTCACCCGGCGG
CGCCCTCCTCCTGGGGGCCCAGGCCCCGCCGCTACACGGTGCTGAGCGT
GGGTCCCGGAGGCCTGCGCAGCGGGAGGCTGCCCCTGCAGCCCCGCGTC
CAGCTGGATGAGCGCGGCCGGCAGCGCGGGGACTTCTCGCTATGGCTGC
GCCCAGCCCGGCGCGCGGACGCCGGCGAGTACCGCGCCGCGGTGCACCT
CAGGGACCGCGCCCTCTCCTGCCGCCTCCGTCTGCGCCTGGGCCAGGCC
TCGATGACTGCCAGCCCCCCAGGATCTCTCAGAGCCTCCGACTGGGTCA
TTTTGAACTGCTCCTTCAGCCGCCCTGACCGCCCAGCCTCTGTGCATTG
GTTCCGGAACCGGGGCCAGGGCCGAGTCCCTGTCCGGGAGTCCCCCCAT
CACCACTTAGCGGAAAGCTTCCTCTTCCTGCCCCAAGTCAGCCCCATGG
ACTCTGGGCCCTGGGGCTGCATCCTCACCTACAGAGATGGCTTCAACGT
CTCCATCATGTATAACCTCACTGTTCTGGGTCTGGAGCCCCCAACTCCC
TTGACAGTGTACGCTGGAGCAGGTTCCAGGGTGGGGCTGCCCTGCCGCC
TGCCTGCTGGTGTGGGGACCCGGTCTTTCCTCACTGCCAAGTGGACTCC
TCCTGGGGGAGGCCCTGACCTCCTGGTGACTGGAGACAATGGCGACTTT
ACCCTTCGACTAGAGGATGTGAGCCAGGCCCAGGCTGGGACCTACACCT
GCCATATCCATCTGCAGGAACAGCAGCTCAATGCCACTGTCACATTGGC
AATCATCACAGTGACTCCCAAATCCTTTGGGTCACCTGGATCCCTGGGG
AAGCTGCTTTGTGAGGTGACTCCAGTATCTGGACAAGAACGCTTTGTGT
GGAGCTCTCTGGACACCCCATCCCAGAGGAGTTTCTCAGGACCTTGGCT
GGAGGCACAGGAGGCCCAGCTCCTTTCCCAGCCTTGGCAATGCCAGCTG
TACCAGGGGGAGAGGCTTCTTGGAGCAGCAGTGTACTTCACAGAGCTGT
CTAGCCCAGGTGCCCAACGCTCTGGGAGAGCCCCAGGTGCCCTCCCAGC
AGGCCACCTCATTCCGTGGCTCGGCCACCTCCTCGTGGGCCTCAGCGGG
GCTTTTGGCTTCATCATCTTAGTGTACTTGCTGATCAACTGCAGGAACA
CCGGGCCATGGCTGAAGAAGGTCCTGAAGTGTAACACCCCAGACCCCTC
GAAGTTCTTTTCCCAGCTGAGCTCAGAGCATGGAGGAGACGTCCAGAAG
TGGCTCTCTTCGCCCTTCCCCTCATCGTCCTTCAGCCCTGGCGGCCTGG
CACCTGAGATCTCGCCACTAGAAGTGCTGGAGAGGGACAAGGTGACGCA
GCTGCTCCTGCAGCAGGACAAGGTGCCTGAGCCCGCATCCTTAAGCAGC
AACCACTCGCTGACCAGCTGCTTCACCAACCAGGGTTACTTCTTCTTCC
ACCTCCCGGATGCCTTGGAGATAGAGGCCTGCCAGGTGTACTTTACTTA
CGACCCCTACTCAGAGGAAGACCCTGATGAGGGTGTGGCCGGGGCACCC
ACAGGGTCTTCCCCCCAACCCCTGCAGCCTCTGTCAGGGGAGGACGACG
CCTACTGCACCTTCCCCTCCAGGGATGACCTGCTGCTCTTCTCCCCCAG
TCTCCTCGGTGGCCCCAGCCCCCCAAGCACTGCCCCTGGGGGCAGTGGG
GCCGGTGAAGAGAGGATGCCCCCTTCTTTGCAAGAAAGAGTCCCCAGAG
ACTGGGACCCCCAGCCCCTGGGGCCTCCCACCCCAGGAGTCCCAGACCT
GGTGGATTTTCAGCCACCCCCTGAGCTGGTGCTGCGAGAGGCTGGGGAG
GAGGTCCCTGACGCTGGCCCCAGGGAGGGAGTCAGTTTCCCCTGGTCCA
GGCCTCCTGGGCAGGGGGAGTTCAGGGCCCTTAATGCTCGCCTGCCCCT
GAACACTGATGCCTACTTGTCCCTCCAAGAACTCCAGGGTCAGGACCCA
ACTCACTTGGTGTAG.

Therefore, in some embodiments, the chimeric receptor is a LAG3-IL21R fusion and has the amino acid sequence:

(SEQ ID NO: 11)
LQPGAEVPVVWAQEGAPAQLPCSPTIPLQDLSLLRRAGVTWQHQPDSGP
PAAAPGHPLAPGPHPAAPSSWGPRPRRYTVLSVGPGGLRSGRLPLQPRV
QLDERGRQRGDFSLWLRPARRADAGEYRAAVHLRDRALSCRLRLRLGQA
SMTASPPGSLRASDWVILNCSFSRPDRPASVHWFRNRGQGRVPVRESPH
HHLAESFLFLPQVSPMDSGPWGCILTYRDGFNVSIMYNLTVLGLEPPTP
LTVYAGAGSRVGLPCRLPAGVGTRSFLTAKWTPPGGGPDLLVTGDNGDF
TLRLEDVSQAQAGTYTCHIHLQEQQLNATVTLAIITVTPKSFGSPGSLG
KLLCEVTPVSGQERFVWSSLDTPSQRSFSGPWLEAQEAQLLSQPWQCQL
YQGERLLGAAVYFTELSSPGAQRSGRAPGALPAGHLGWNPHLLLLLLLV
IVFIPAFWSLKTHPLWRLWKKIWAVPSPERFFMPLYKGCSGDFKKWVGA
PFTGSSLELGPWSPEVPSTLEVYSCHPPRSPAKRLQLTELQEPAELVES
DGVPKPSFWPTAQNSGGSAYSEERDRPYGLVSIDTVTVLDAEGPCTWPC
SCEDDGYPALDLDAGLEPSPGLEDPLLDAGTTVLSCGCVSAGSPGLGGP
LGSLLDRLKPPLADGEDWAGGLPWGGRSPGGVSESEAGSPLAGLDMDTF
DSGFVGSDCSSPVECDFTSPGDEGPPRSYLRQWVVIPPPLSSPGPQAS.

Therefore, in some embodiments, the chimeric receptor is encoded by the

(SEQ ID NO: 43)
CTCCAGCCAGGGGCTGAGGTCCCGGTGGTGTGGGCCCAGGAGGGGGCTC
CTGCCCAGCTCCCCTGCAGCCCCACAATCCCCCTCCAGGATCTCAGCCT
TCTGCGAAGAGCAGGGGTCACTTGGCAGCATCAGCCAGACAGTGGCCCG
CCCGCTGCCGCCCCCGGCCATCCCCTGGCCCCCGGCCCTCACCCGGCGG
CGCCCTCCTCCTGGGGGCCCAGGCCCCGCCGCTACACGGTGCTGAGCGT
GGGTCCCGGAGGCCTGCGCAGCGGGAGGCTGCCCCTGCAGCCCCGCGTC
CAGCTGGATGAGCGCGGCCGGCAGCGCGGGGACTTCTCGCTATGGCTGC
GCCCAGCCCGGCGCGCGGACGCCGGCGAGTACCGCGCCGCGGTGCACCT
CAGGGACCGCGCCCTCTCCTGCCGCCTCCGTCTGCGCCTGGGCCAGGCC
TCGATGACTGCCAGCCCCCCAGGATCTCTCAGAGCCTCCGACTGGGTCA
TTTTGAACTGCTCCTTCAGCCGCCCTGACCGCCCAGCCTCTGTGCATTG
GTTCCGGAACCGGGGCCAGGGCCGAGTCCCTGTCCGGGAGTCCCCCCAT
CACCACTTAGCGGAAAGCTTCCTCTTCCTGCCCCAAGTCAGCCCCATGG
ACTCTGGGCCCTGGGGCTGCATCCTCACCTACAGAGATGGCTTCAACGT
CTCCATCATGTATAACCTCACTGTTCTGGGTCTGGAGCCCCCAACTCCC
TTGACAGTGTACGCTGGAGCAGGTTCCAGGGTGGGGCTGCCCTGCCGCC
TGCCTGCTGGTGTGGGGACCCGGTCTTTCCTCACTGCCAAGTGGACTCC
TCCTGGGGGAGGCCCTGACCTCCTGGTGACTGGAGACAATGGCGACTTT
ACCCTTCGACTAGAGGATGTGAGCCAGGCCCAGGCTGGGACCTACACCT
GCCATATCCATCTGCAGGAACAGCAGCTCAATGCCACTGTCACATTGGC
AATCATCACAGTGACTCCCAAATCCTTTGGGTCACCTGGATCCCTGGGG
AAGCTGCTTTGTGAGGTGACTCCAGTATCTGGACAAGAACGCTTTGTGT
GGAGCTCTCTGGACACCCCATCCCAGAGGAGTTTCTCAGGACCTTGGCT
GGAGGCACAGGAGGCCCAGCTCCTTTCCCAGCCTTGGCAATGCCAGCTG
TACCAGGGGGAGAGGCTTCTTGGAGCAGCAGTGTACTTCACAGAGCTGT
CTAGCCCAGGTGCCCAACGCTCTGGGAGAGCCCCAGGTGCCCTCCCAGC
AGGCCACCTCGGCTGGAACCCTCACCTGCTGCTTCTCCTCCTGCTTGTC
ATAGTCTTCATTCCTGCCTTCTGGAGCCTGAAGACCCATCCATTGTGGA
GGCTATGGAAGAAGATATGGGCCGTCCCCAGCCCTGAGCGGTTCTTCAT
GCCCCTGTACAAGGGCTGCAGCGGAGACTTCAAGAAATGGGTGGGTGCA
CCCTTCACTGGCTCCAGCCTGGAGCTGGGACCCTGGAGCCCAGAGGTGC
CCTCCACCCTGGAGGTGTACAGCTGCCACCCACCACGGAGCCCGGCCAA
GAGGCTGCAGCTCACGGAGCTACAAGAACCAGCAGAGCTGGTGGAGTCT
GACGGTGTGCCCAAGCCCAGCTTCTGGCCGACAGCCCAGAACTCGGGGG
GCTCAGCTTACAGTGAGGAGAGGGATCGGCCATACGGCCTGGTGTCCAT
TGACACAGTGACTGTGCTAGATGCAGAGGGGCCATGCACCTGGCCCTGC
AGCTGTGAGGATGACGGCTACCCAGCCCTGGACCTGGATGCTGGCCTGG
AGCCCAGCCCAGGCCTAGAGGACCCACTCTTGGATGCAGGGACCACAGT
CCTGTCCTGTGGCTGTGTCTCAGCTGGCAGCCCTGGGCTAGGAGGGCCC
CTGGGAAGCCTCCTGGACAGACTAAAGCCACCCCTTGCAGATGGGGAGG
ACTGGGCTGGGGGACTGCCCTGGGGTGGCCGGTCACCTGGAGGGGTCTC
AGAGAGTGAGGCGGGCTCACCCCTGGCCGGCCTGGATATGGACACGTTT
GACAGTGGCTTTGTGGGCTCTGACTGCAGCAGCCCTGTGGAGTGTGACT
TCACCAGCCCCGGGGACGAAGGACCCCCCCGGAGCTACCTCCGCCAGTG
GGTGGTCATTCCTCCGCCACTTTCGAGCCCTGGACCCCAGGCCAGCTAA
TGA.

Therefore, in some embodiments, the chimeric receptor is a LAG3-IL12Rb2 fusion and has the amino acid sequence:

(SEQ ID NO: 12)
LQPGAEVPVVWAQEGAPAQLPCSPTIPLQDLSLLRRAGVTWQHQPDSGP
PAAAPGHPLAPGPHPAAPSSWGPRPRRYTVLSVGPGGLRSGRLPLQPRV
QLDERGRQRGDFSLWLRPARRADAGEYRAAVHLRDRALSCRLRLRLGQA
SMTASPPGSLRASDWVILNCSFSRPDRPASVHWFRNRGQGRVPVRESPH
HHLAESFLFLPQVSPMDSGPWGCILTYRDGFNVSIMYNLTVLGLEPPTP
LTVYAGAGSRVGLPCRLPAGVGTRSFLTAKWTPPGGGPDLLVTGDNGDF
TLRLEDVSQAQAGTYTCHIHLQEQQLNATVTLAIITVTPKSFGSPGSLG
KLLCEVTPVSGQERFVWSSLDTPSQRSFSGPWLEAQEAQLLSQPWQCQL
YQGERLLGAAVYFTELSSPGAQRSGRAPGALPAGHLWMAFVAPSICIAI
IMVGIFSTHYFQQKVFVLLAALRPQWCSREIPDPANSTCAKKYPIAEEK
TQLPLDRLLIDWPTPEDPEPLVISEVLHQVTPVFRHPPCSNWPQREKGI
QGHQASEKDMMHSASSPPPPRALQAESRQLVDLYKVLESRGSDPKPENP
ACPWTVLPAGDLPTHDGYLPSNIDDLPSHEAPLADSLEELEPQHISLSV
FPSSSLHPLTFSCGDKLTLDQLKMRCDSLML.

Therefore, in some embodiments, the chimeric receptor is encoded by the

(SEQ ID NO: 44)
CTCCAGCCAGGGGCTGAGGTCCCGGTGGTGTGGGCCCAGGAGGGGGCTC
CTGCCCAGCTCCCCTGCAGCCCCACAATCCCCCTCCAGGATCTCAGCCT
TCTGCGAAGAGCAGGGGTCACTTGGCAGCATCAGCCAGACAGTGGCCCG
CCCGCTGCCGCCCCCGGCCATCCCCTGGCCCCCGGCCCTCACCCGGCGG
CGCCCTCCTCCTGGGGGCCCAGGCCCCGCCGCTACACGGTGCTGAGCGT
GGGTCCCGGAGGCCTGCGCAGCGGGAGGCTGCCCCTGCAGCCCCGCGTC
CAGCTGGATGAGCGCGGCCGGCAGCGCGGGGACTTCTCGCTATGGCTGC
GCCCAGCCCGGCGCGCGGACGCCGGCGAGTACCGCGCCGCGGTGCACCT
CAGGGACCGCGCCCTCTCCTGCCGCCTCCGTCTGCGCCTGGGCCAGGCC
TCGATGACTGCCAGCCCCCCAGGATCTCTCAGAGCCTCCGACTGGGTCA
TTTTGAACTGCTCCTTCAGCCGCCCTGACCGCCCAGCCTCTGTGCATTG
GTTCCGGAACCGGGGCCAGGGCCGAGTCCCTGTCCGGGAGTCCCCCCAT
CACCACTTAGCGGAAAGCTTCCTCTTCCTGCCCCAAGTCAGCCCCATGG
ACTCTGGGCCCTGGGGCTGCATCCTCACCTACAGAGATGGCTTCAACGT
CTCCATCATGTATAACCTCACTGTTCTGGGTCTGGAGCCCCCAACTCCC
TTGACAGTGTACGCTGGAGCAGGTTCCAGGGTGGGGCTGCCCTGCCGCC
TGCCTGCTGGTGTGGGGACCCGGTCTTTCCTCACTGCCAAGTGGACTCC
TCCTGGGGGAGGCCCTGACCTCCTGGTGACTGGAGACAATGGCGACTTT
ACCCTTCGACTAGAGGATGTGAGCCAGGCCCAGGCTGGGACCTACACCT
GCCATATCCATCTGCAGGAACAGCAGCTCAATGCCACTGTCACATTGGC
AATCATCACAGTGACTCCCAAATCCTTTGGGTCACCTGGATCCCTGGGG
AAGCTGCTTTGTGAGGTGACTCCAGTATCTGGACAAGAACGCTTTGTGT
GGAGCTCTCTGGACACCCCATCCCAGAGGAGTTTCTCAGGACCTTGGCT
GGAGGCACAGGAGGCCCAGCTCCTTTCCCAGCCTTGGCAATGCCAGCTG
TACCAGGGGGAGAGGCTTCTTGGAGCAGCAGTGTACTTCACAGAGCTGT
CTAGCCCAGGTGCCCAACGCTCTGGGAGAGCCCCAGGTGCCCTCCCAGC
AGGCCACCTCTGGATGGCGTTTGTGGCACCAAGCATTTGCATTGCTATC
ATCATGGTGGGCATTTTCTCAACGCATTACTTCCAGCAAAAGGTGTTTG
TTCTCCTAGCAGCCCTCAGACCTCAGTGGTGTAGCAGAGAAATTCCAGA
TCCAGCAAATAGCACTTGCGCTAAGAAATATCCCATTGCAGAGGAGAAG
ACACAGCTGCCCTTGGACAGGCTCCTGATAGACTGGCCCACGCCTGAAG
ATCCTGAACCGCTGGTCATCAGTGAAGTCCTTCATCAAGTGACCCCAGT
TTTCAGACATCCCCCCTGCTCCAACTGGCCACAAAGGGAAAAAGGAATC
CAAGGTCATCAGGCCTCTGAGAAAGACATGATGCACAGTGCCTCAAGCC
CACCACCTCCAAGAGCTCTCCAAGCTGAGAGCAGACAACTGGTGGATCT
GTACAAGGTGCTGGAGAGCAGGGGCTCCGACCCAAAGCCAGAAAACCCA
GCCTGTCCCTGGACGGTGCTCCCAGCAGGTGACCTTCCCACCCATGATG
GCTACTTACCCTCCAACATAGATGACCTCCCCTCACATGAGGCACCTCT
CGCTGACTCTCTGGAAGAACTGGAGCCTCAGCACATCTCCCTTTCTGTT
TTCCCCTCAAGTTCTCTTCACCCACTCACCTTCTCCTGTGGTGATAAGC
TGACTCTGGATCAGTTAAAGATGAGGTGTGACTCCCTCATGCTCTGA.

Therefore, in some embodiments, the chimeric receptor is a LAG3-IL6ST fusion and has the amino acid sequence:

(SEQ ID NO: 13)
LQPGAEVPVVWAQEGAPAQLPCSPTIPLQDLSLLRRAGVTWQHQPDSGP
PAAAPGHPLAPGPHPAAPSSWGPRPRRYTVLSVGPGGLRSGRLPLQPRV
QLDERGRQRGDFSLWLRPARRADAGEYRAAVHLRDRALSCRLRLRLGQA
SMTASPPGSLRASDWVILNCSFSRPDRPASVHWFRNRGQGRVPVRESPH
HHLAESFLFLPQVSPMDSGPWGCILTYRDGFNVSIMYNLTVLGLEPPTP
LTVYAGAGSRVGLPCRLPAGVGTRSFLTAKWTPPGGGPDLLVTGDNGDF
TLRLEDVSQAQAGTYTCHIHLQEQQLNATVTLAIITVTPKSFGSPGSLG
KLLCEVTPVSGQERFVWSSLDTPSQRSFSGPWLEAQEAQLLSQPWQCQL
YQGERLLGAAVYFTELSSPGAQRSGRAPGALPAGHLAIVVPVCLAFLLT
TLLGVLFCFNKRDLIKKHIWPNVPDPSKSHIAQWSPHTPPRHNFNSKDQ
MYSDGNFTDVSVVEIEANDKKPFPEDLKSLDLFKKEKINTEGHSSGIGG
SSCMSSSRPSISSSDENESSQNTSSTVQYSTVVHSGYRHQVPSVQVFSR
SESTQPLLDSEERPEDLQLVDHVDGGDGILPRQQYFKQNCSQHESSPDI
SHFERSKQVSSVNEEDFVRLKQQISDHISQSCGSGQMKMFQEVSAADAF
GPGTEGQVERFETVGMEAATDEGMPKSYLPQTVRQGGYMPQ.

Therefore, in some embodiments, the chimeric receptor is encoded by the

(SEQ ID NO: 45)
CTCCAGCCAGGGGCTGAGGTCCCGGTGGTGTGGGCCCAGGAGGGGGCTC
CTGCCCAGCTCCCCTGCAGCCCCACAATCCCCCTCCAGGATCTCAGCCT
TCTGCGAAGAGCAGGGGTCACTTGGCAGCATCAGCCAGACAGTGGCCCG
CCCGCTGCCGCCCCCGGCCATCCCCTGGCCCCCGGCCCTCACCCGGCGG
CGCCCTCCTCCTGGGGGCCCAGGCCCCGCCGCTACACGGTGCTGAGCGT
GGGTCCCGGAGGCCTGCGCAGCGGGAGGCTGCCCCTGCAGCCCCGCGTC
CAGCTGGATGAGCGCGGCCGGCAGCGCGGGGACTTCTCGCTATGGCTGC
GCCCAGCCCGGCGCGCGGACGCCGGCGAGTACCGCGCCGCGGTGCACCT
CAGGGACCGCGCCCTCTCCTGCCGCCTCCGTCTGCGCCTGGGCCAGGCC
TCGATGACTGCCAGCCCCCCAGGATCTCTCAGAGCCTCCGACTGGGTCA
TTTTGAACTGCTCCTTCAGCCGCCCTGACCGCCCAGCCTCTGTGCATTG
GTTCCGGAACCGGGGCCAGGGCCGAGTCCCTGTCCGGGAGTCCCCCCAT
CACCACTTAGCGGAAAGCTTCCTCTTCCTGCCCCAAGTCAGCCCCATGG
ACTCTGGGCCCTGGGGCTGCATCCTCACCTACAGAGATGGCTTCAACGT
CTCCATCATGTATAACCTCACTGTTCTGGGTCTGGAGCCCCCAACTCCC
TTGACAGTGTACGCTGGAGCAGGTTCCAGGGTGGGGCTGCCCTGCCGCC
TGCCTGCTGGTGTGGGGACCCGGTCTTTCCTCACTGCCAAGTGGACTCC
TCCTGGGGGAGGCCCTGACCTCCTGGTGACTGGAGACAATGGCGACTTT
ACCCTTCGACTAGAGGATGTGAGCCAGGCCCAGGCTGGGACCTACACCT
GCCATATCCATCTGCAGGAACAGCAGCTCAATGCCACTGTCACATTGGC
AATCATCACAGTGACTCCCAAATCCTTTGGGTCACCTGGATCCCTGGGG
AAGCTGCTTTGTGAGGTGACTCCAGTATCTGGACAAGAACGCTTTGTGT
GGAGCTCTCTGGACACCCCATCCCAGAGGAGTTTCTCAGGACCTTGGCT
GGAGGCACAGGAGGCCCAGCTCCTTTCCCAGCCTTGGCAATGCCAGCTG
TACCAGGGGGAGAGGCTTCTTGGAGCAGCAGTGTACTTCACAGAGCTGT
CTAGCCCAGGTGCCCAACGCTCTGGGAGAGCCCCAGGTGCCCTCCCAGC
AGGCCACCTCGCCATAGTCGTGCCTGTTTGCTTAGCATTCCTATTGACA
ACTCTTCTGGGAGTGCTGTTCTGCTTTAATAAGCGAGACCTAATTAAAA
AACACATCTGGCCTAATGTTCCAGATCCTTCAAAGAGTCATATTGCCCA
GTGGTCACCTCACACTCCTCCAAGGCACAATTTTAATTCAAAAGATCAA
ATGTATTCAGATGGCAATTTCACTGATGTAAGTGTTGTGGAAATAGAAG
CAAATGACAAAAAGCCTTTTCCAGAAGATCTGAAATCATTGGACCTGTT
CAAAAAGGAAAAAATTAATACTGAAGGACACAGCAGTGGTATTGGGGGG
TCTTCATGCATGTCATCTTCTAGGCCAAGCATTTCTAGCAGTGATGAAA
ATGAATCTTCACAAAACACTTCGAGCACTGTCCAGTATTCTACCGTGGT
ACACAGTGGCTACAGACACCAAGTTCCGTCAGTCCAAGTCTTCTCAAGA
TCCGAGTCTACCCAGCCCTTGTTAGATTCAGAGGAGCGGCCAGAAGATC
TACAATTAGTAGATCATGTAGATGGCGGTGATGGTATTTTGCCCAGGCA
ACAGTACTTCAAACAGAACTGCAGTCAGCATGAATCCAGTCCAGATATT
TCACATTTTGAAAGGTCAAAGCAAGTTTCATCAGTCAATGAGGAAGATT
TTGTTAGACTTAAACAGCAGATTTCAGATCATATTTCACAATCCTGTGG
ATCTGGGCAAATGAAAATGTTTCAGGAAGTTTCTGCAGCAGATGCTTTT
GGTCCAGGTACTGAGGGACAAGTAGAAAGATTTGAAACAGTTGGCATGG
AGGCTGCGACTGATGAAGGCATGCCTAAAAGTTACTTACCACAGACTGT
ACGGCAAGGCGGCTACATGCCTCAGT.

Therefore, in some embodiments, the chimeric receptor is a LAG3-CD27 fusion and has the amino acid sequence:

(SEQ ID NO: 14)
LQPGAEVPVVWAQEGAPAQLPCSPTIPLQDLSLLRRAGVTWQHQPDSGP
PAAAPGHPLAPGPHPAAPSSWGPRPRRYTVLSVGPGGLRSGRLPLQPRV
QLDERGRQRGDFSLWLRPARRADAGEYRAAVHLRDRALSCRLRLRLGQA
SMTASPPGSLRASDWVILNCSFSRPDRPASVHWFRNRGQGRVPVRESPH
HHLAESFLFLPQVSPMDSGPWGCILTYRDGFNVSIMYNLTVLGLEPPTP
LTVYAGAGSRVGLPCRLPAGVGTRSFLTAKWTPPGGGPDLLVTGDNGDF
TLRLEDVSQAQAGTYTCHIHLQEQQLNATVTLAIITVTPKSFGSPGSLG
KLLCEVTPVSGQERFVWSSLDTPSQRSFSGPWLEAQEAQLLSQPWQCQL
YQGERLLGAAVYFTELSSPGAQRSGRAPGALPAGHLILVIFSGMFLVFT
LAGALFLHQRRKYRSNKGESPVEPAEPCRYSCPREEEGSTIPIQEDYRK
PEPACSP.

Therefore, in some embodiments, the chimeric receptor is encoded by the

(SEQ ID NO: 46)
CTCCAGCCAGGGGCTGAGGTCCCGGTGGTGTGGGCCCAGGAGGGGGCTCC
TGCCCAGCTCCCCTGCAGCCCCACAATCCCCCTCCAGGATCTCAGCCTTC
TGCGAAGAGCAGGGGTCACTTGGCAGCATCAGCCAGACAGTGGCCCGCCC
GCTGCCGCCCCCGGCCATCCCCTGGCCCCCGGCCCTCACCCGGCGGCGCC
CTCCTCCTGGGGGCCCAGGCCCCGCCGCTACACGGTGCTGAGCGTGGGTC
CCGGAGGCCTGCGCAGCGGGAGGCTGCCCCTGCAGCCCCGCGTCCAGCTG
GATGAGCGCGGCCGGCAGCGCGGGGACTTCTCGCTATGGCTGCGCCCAGC
CCGGCGCGCGGACGCCGGCGAGTACCGCGCCGCGGTGCACCTCAGGGACC
GCGCCCTCTCCTGCCGCCTCCGTCTGCGCCTGGGCCAGGCCTCGATGACT
GCCAGCCCCCCAGGATCTCTCAGAGCCTCCGACTGGGTCATTTTGAACTG
CTCCTTCAGCCGCCCTGACCGCCCAGCCTCTGTGCATTGGTTCCGGAACC
GGGGCCAGGGCCGAGTCCCTGTCCGGGAGTCCCCCCATCACCACTTAGCG
GAAAGCTTCCTCTTCCTGCCCCAAGTCAGCCCCATGGACTCTGGGCCCTG
GGGCTGCATCCTCACCTACAGAGATGGCTTCAACGTCTCCATCATGTATA
ACCTCACTGTTCTGGGTCTGGAGCCCCCAACTCCCTTGACAGTGTACGCT
GGAGCAGGTTCCAGGGTGGGGCTGCCCTGCCGCCTGCCTGCTGGTGTGGG
GACCCGGTCTTTCCTCACTGCCAAGTGGACTCCTCCTGGGGGAGGCCCTG
ACCTCCTGGTGACTGGAGACAATGGCGACTTTACCCTTCGACTAGAGGAT
GTGAGCCAGGCCCAGGCTGGGACCTACACCTGCCATATCCATCTGCAGGA
ACAGCAGCTCAATGCCACTGTCACATTGGCAATCATCACAGTGACTCCCA
AATCCTTTGGGTCACCTGGATCCCTGGGGAAGCTGCTTTGTGAGGTGACT
CCAGTATCTGGACAAGAACGCTTTGTGTGGAGCTCTCTGGACACCCCATC
CCAGAGGAGTTTCTCAGGACCTTGGCTGGAGGCACAGGAGGCCCAGCTCC
TTTCCCAGCCTTGGCAATGCCAGCTGTACCAGGGGGAGAGGCTTCTTGGA
GCAGCAGTGTACTTCACAGAGCTGTCTAGCCCAGGTGCCCAACGCTCTGG
GAGAGCCCCAGGTGCCCTCCCAGCAGGCCACCTCATCCTTGTGATCTTCT
CTGGAATGTTCCTTGTTTTCACCCTGGCCGGGGCCCTGTTCCTCCATCAA
CGAAGGAAATATAGATCAAACAAAGGAGAAAGTCCTGTGGAGCCTGCAGA
GCCTTGTCGTTACAGCTGCCCCAGGGAGGAGGAGGGCAGCACCATCCCCA
TCCAGGAGGATTACCGAAAACCGGAGCCTGCCTGCTCCCCCTGA.

Therefore, in some embodiments, the chimeric receptor is a LAG3-ICOS fusion and has the amino acid sequence:

(SEQ ID NO: 15)
LQPGAEVPVVWAQEGAPAQLPCSPTIPLQDLSLLRRAGVTWQHQPDSGPP
AAAPGHPLAPGPHPAAPSSWGPRPRRYTVLSVGPGGLRSGRLPLQPRVQL
DERGRQRGDFSLWLRPARRADAGEYRAAVHLRDRALSCRLRLRLGQASMT
ASPPGSLRASDWVILNCSFSRPDRPASVHWFRNRGQGRVPVRESPHHHLA
ESFLFLPQVSPMDSGPWGCILTYRDGFNVSIMYNLTVLGLEPPTPLTVYA
GAGSRVGLPCRLPAGVGTRSFLTAKWTPPGGGPDLLVTGDNGDFTLRLED
VSQAQAGTYTCHIHLQEQQLNATVTLAIITVTPKSFGSPGSLGKLLCEVT
PVSGQERFVWSSLDTPSQRSFSGPWLEAQEAQLLSQPWQCQLYQGERLLG
AAVYFTELSSPGAQRSGRAPGALPAGHLFWLPIGCAAFVVVCILGCILIC
WLTKKKYSSSVHDPNGEYMFMRAVNTAKKSRLTDVTL.

Therefore, in some embodiments, the chimeric receptor is encoded by the nucleic acid sequence:

(SEQ ID NO: 47)
CTCCAGCCAGGGGCTGAGGTCCCGGTGGTGTGGGCCCAGGAGGGGGCTCC
TGCCCAGCTCCCCTGCAGCCCCACAATCCCCCTCCAGGATCTCAGCCTTC
TGCGAAGAGCAGGGGTCACTTGGCAGCATCAGCCAGACAGTGGCCCGCCC
GCTGCCGCCCCCGGCCATCCCCTGGCCCCCGGCCCTCACCCGGCGGCGCC
CTCCTCCTGGGGGCCCAGGCCCCGCCGCTACACGGTGCTGAGCGTGGGTC
CCGGAGGCCTGCGCAGCGGGAGGCTGCCCCTGCAGCCCCGCGTCCAGCTG
GATGAGCGCGGCCGGCAGCGCGGGGACTTCTCGCTATGGCTGCGCCCAGC
CCGGCGCGCGGACGCCGGCGAGTACCGCGCCGCGGTGCACCTCAGGGACC
GCGCCCTCTCCTGCCGCCTCCGTCTGCGCCTGGGCCAGGCCTCGATGACT
GCCAGCCCCCCAGGATCTCTCAGAGCCTCCGACTGGGTCATTTTGAACTG
CTCCTTCAGCCGCCCTGACCGCCCAGCCTCTGTGCATTGGTTCCGGAACC
GGGGCCAGGGCCGAGTCCCTGTCCGGGAGTCCCCCCATCACCACTTAGCG
GAAAGCTTCCTCTTCCTGCCCCAAGTCAGCCCCATGGACTCTGGGCCCTG
GGGCTGCATCCTCACCTACAGAGATGGCTTCAACGTCTCCATCATGTATA
ACCTCACTGTTCTGGGTCTGGAGCCCCCAACTCCCTTGACAGTGTACGCT
GGAGCAGGTTCCAGGGTGGGGCTGCCCTGCCGCCTGCCTGCTGGTGTGGG
GACCCGGTCTTTCCTCACTGCCAAGTGGACTCCTCCTGGGGGAGGCCCTG
ACCTCCTGGTGACTGGAGACAATGGCGACTTTACCCTTCGACTAGAGGAT
GTGAGCCAGGCCCAGGCTGGGACCTACACCTGCCATATCCATCTGCAGGA
ACAGCAGCTCAATGCCACTGTCACATTGGCAATCATCACAGTGACTCCCA
AATCCTTTGGGTCACCTGGATCCCTGGGGAAGCTGCTTTGTGAGGTGACT
CCAGTATCTGGACAAGAACGCTTTGTGTGGAGCTCTCTGGACACCCCATC
CCAGAGGAGTTTCTCAGGACCTTGGCTGGAGGCACAGGAGGCCCAGCTCC
TTTCCCAGCCTTGGCAATGCCAGCTGTACCAGGGGGAGAGGCTTCTTGGA
GCAGCAGTGTACTTCACAGAGCTGTCTAGCCCAGGTGCCCAACGCTCTGG
GAGAGCCCCAGGTGCCCTCCCAGCAGGCCACCTCTTCTGGTTACCCATAG
GATGTGCAGCCTTTGTTGTAGTCTGCATTTTGGGATGCATACTTATTTGT
TGGCTTACAAAAAAGAAGTATTCATCCAGTGTGCACGACCCTAACGGTGA
ATACATGTTCATGAGAGCAGTGAACACAGCCAAAAAATCTAGACTCACAG
ATGTGACCCTATAA.

Therefore, in some embodiments, the chimeric receptor is a LAG3-OX40 fusion and has the amino acid sequence:

(SEQ ID NO: 16)
LQPGAEVPVVWAQEGAPAQLPCSPTIPLQDLSLLRRAGVTWQHQPDSGPP
AAAPGHPLAPGPHPAAPSSWGPRPRRYTVLSVGPGGLRSGRLPLQPRVQL
DERGRQRGDFSLWLRPARRADAGEYRAAVHLRDRALSCRLRLRLGQASMT
ASPPGSLRASDWVILNCSFSRPDRPASVHWFRNRGQGRVPVRESPHHHLA
ESFLFLPQVSPMDSGPWGCILTYRDGFNVSIMYNLTVLGLEPPTPLTVYA
GAGSRVGLPCRLPAGVGTRSFLTAKWTPPGGGPDLLVTGDNGDFTLRLED
VSQAQAGTYTCHIHLQEQQLNATVTLAIITVTPKSFGSPGSLGKLLCEVT
PVSGQERFVWSSLDTPSQRSFSGPWLEAQEAQLLSQPWQCQLYQGERLLG
AAVYFTELSSPGAQRSGRAPGALPAGHLVAAILGLGLVLGLLGPLAILLA
LYLLRRDQRLPPDAHKPPGGGSFRTPIQEEQADAHSTLAKI.

Therefore, in some embodiments, the chimeric receptor is encoded by the

(SEQ ID NO: 48)
CTCCAGCCAGGGGCTGAGGTCCCGGTGGTGTGGGCCCAGGAGGGGGCTCC
TGCCCAGCTCCCCTGCAGCCCCACAATCCCCCTCCAGGATCTCAGCCTTC
TGCGAAGAGCAGGGGTCACTTGGCAGCATCAGCCAGACAGTGGCCCGCCC
GCTGCCGCCCCCGGCCATCCCCTGGCCCCCGGCCCTCACCCGGCGGCGCC
CTCCTCCTGGGGGCCCAGGCCCCGCCGCTACACGGTGCTGAGCGTGGGTC
CCGGAGGCCTGCGCAGCGGGAGGCTGCCCCTGCAGCCCCGCGTCCAGCTG
GATGAGCGCGGCCGGCAGCGCGGGGACTTCTCGCTATGGCTGCGCCCAGC
CCGGCGCGCGGACGCCGGCGAGTACCGCGCCGCGGTGCACCTCAGGGACC
GCGCCCTCTCCTGCCGCCTCCGTCTGCGCCTGGGCCAGGCCTCGATGACT
GCCAGCCCCCCAGGATCTCTCAGAGCCTCCGACTGGGTCATTTTGAACTG
CTCCTTCAGCCGCCCTGACCGCCCAGCCTCTGTGCATTGGTTCCGGAACC
GGGGCCAGGGCCGAGTCCCTGTCCGGGAGTCCCCCCATCACCACTTAGCG
GAAAGCTTCCTCTTCCTGCCCCAAGTCAGCCCCATGGACTCTGGGCCCTG
GGGCTGCATCCTCACCTACAGAGATGGCTTCAACGTCTCCATCATGTATA
ACCTCACTGTTCTGGGTCTGGAGCCCCCAACTCCCTTGACAGTGTACGCT
GGAGCAGGTTCCAGGGTGGGGCTGCCCTGCCGCCTGCCTGCTGGTGTGGG
GACCCGGTCTTTCCTCACTGCCAAGTGGACTCCTCCTGGGGGAGGCCCTG
ACCTCCTGGTGACTGGAGACAATGGCGACTTTACCCTTCGACTAGAGGAT
GTGAGCCAGGCCCAGGCTGGGACCTACACCTGCCATATCCATCTGCAGGA
ACAGCAGCTCAATGCCACTGTCACATTGGCAATCATCACAGTGACTCCCA
AATCCTTTGGGTCACCTGGATCCCTGGGGAAGCTGCTTTGTGAGGTGACT
CCAGTATCTGGACAAGAACGCTTTGTGTGGAGCTCTCTGGACACCCCATC
CCAGAGGAGTTTCTCAGGACCTTGGCTGGAGGCACAGGAGGCCCAGCTCC
TTTCCCAGCCTTGGCAATGCCAGCTGTACCAGGGGGAGAGGCTTCTTGGA
GCAGCAGTGTACTTCACAGAGCTGTCTAGCCCAGGTGCCCAACGCTCTGG
GAGAGCCCCAGGTGCCCTCCCAGCAGGCCACCTCGTTGCCGCCATCCTGG
GCCTGGGCCTGGTGCTGGGGCTGCTGGGCCCCCTGGCCATCCTGCTGGCC
CTGTACCTGCTCCGGAGGGACCAGAGGCTGCCCCCCGATGCCCACAAGCC
CCCTGGGGGAGGCAGTTTCCGGACCCCCATCCAAGAGGAGCAGGCCGACG
CCCACTCCACCCTGGCCAAGATCTGA.

Therefore, in some embodiments, the chimeric receptor is a CD112R-IL7Rα fusion and has the amino acid sequence:

(SEQ ID NO: 17)
TPEVWVQVRMEATELSSFTIRCGFLGSGSISLVTVSWGGPNGAGGTTLAV
LHPERGIRQWAPARQARWETQSSISLILEGSGASSPCANTTFCCKFASFP
EGSWEACGSLPPSSDPGLSAPPTPAPILRADLPILLTISILSFFSVALLV
ILACVLWKKRIKPIVWPSLPDHKKTLEHLCKKPRKNLNVSFNPESFLDCQ
IHRVDDIQARDEVEGFLQDTFPQQLEESEKQRLGGDVQSPNCPSEDVVTP
ESFGRDSSLTCLAGNVSACDAPILSSSRSLDCRESGKNGPHVYQDLLLSL
GTTNSTLPPPFSLQSGILTLNPVAQGQPILTSLGSNQEEAYVTMSSFYQN
Q.

Therefore, in some embodiments, the chimeric receptor is encoded by the

(SEQ ID NO: 49)
ACCCCGGAGGTGTGGGTTCAAGTTCGGATGGAGGCCACCGAGCTCTCGTC
CTTCACCATCCGTTGTGGGTTCCTGGGGTCTGGCTCCATCTCCCTGGTGA
CTGTGAGCTGGGGGGGCCCCAACGGTGCTGGGGGGACCACGCTGGCTGTG
TTGCACCCAGAACGTGGCATCCGGCAATGGGCCCCTGCTCGCCAGGCCCG
CTGGGAAACCCAGAGCAGCATCTCTCTCATCCTGGAAGGCTCTGGGGCCA
GCAGCCCCTGCGCCAACACCACCTTCTGCTGCAAGTTTGCGTCCTTCCCT
GAGGGCTCCTGGGAGGCCTGTGGGAGCCTCCCGCCCAGCTCAGACCCAGG
GCTCTCTGCCCCGCCGACTCCTGCCCCCATTCTGCGGGCAGACCTGCCTA
TCTTACTAACCATCAGCATTTTGAGTTTTTTCTCTGTCGCTCTGTTGGTC
ATCTTGGCCTGTGTGTTATGGAAAAAAAGGATTAAGCCTATCGTATGGCC
CAGTCTCCCCGATCATAAGAAGACTCTGGAACATCTTTGTAAGAAACCAA
GAAAAAATTTAAATGTGAGTTTCAATCCTGAAAGTTTCCTGGACTGCCAG
ATTCATAGGGTGGATGACATTCAAGCTAGAGATGAAGTGGAAGGTTTTCT
GCAAGATACGTTTCCTCAGCAACTAGAAGAATCTGAGAAGCAGAGGCTTG
GAGGGGATGTGCAGAGCCCCAACTGCCCATCTGAGGATGTAGTCGTCACT
CCAGAAAGCTTTGGAAGAGATTCATCCCTCACATGCCTGGCTGGGAATGT
CAGTGCATGTGACGCCCCTATTCTCTCCTCTTCCAGGTCCCTAGACTGCA
GGGAGAGTGGCAAGAATGGGCCTCATGTGTACCAGGACCTCCTGCTTAGC
CTTGGGACTACAAACAGCACGCTGCCCCCTCCATTTTCTCTCCAATCTGG
AATCCTGACATTGAACCCAGTTGCTCAGGGTCAGCCCATTCTTACTTCCC
TGGGATCAAATCAAGAAGAAGCATATGTCACCATGTCCAGCTTCTACCAA
AACCAGTGA.

Therefore, in some embodiments, the chimeric receptor is a CD112R-β/IL2, 15 fusion and has the amino acid sequence:

(SEQ ID NO: 18)
TPEVWVQVRMEATELSSFTIRCGFLGSGSISLVTVSWGGPNGAGGTTLAV
LHPERGIRQWAPARQARWETQSSISLILEGSGASSPCANTTFCCKFASFP
EGSWEACGSLPPSSDPGLSAPPTPAPILRADLIPWLGHLLVGLSGAFGFI
ILVYLLINCRNTGPWLKKVLKCNTPDPSKFFSQLSSEHGGDVQKWLSSPF
PSSSFSPGGLAPEISPLEVLERDKVTQLLLQQDKVPEPASLSSNHSLTSC
FTNQGYFFFHLPDALEIEACQVYFTYDPYSEEDPDEGVAGAPTGSSPQPL
QPLSGEDDAYCTFPSRDDLLLFSPSLLGGPSPPSTAPGGSGAGEERMPPS
LQERVPRDWDPQPLGPPTPGVPDLVDFQPPPELVLREAGEEVPDAGPREG
VSFPWSRPPGQGEFRALNARLPLNTDAYLSLQELQGQDPTHLV.

Therefore, in some embodiments, the chimeric receptor is encoded by the

(SEQ ID NO: 50)
ACCCCGGAGGTGTGGGTTCAAGTTCGGATGGAGGCCACCGAGCTCTCGTC
CTTCACCATCCGTTGTGGGTTCCTGGGGTCTGGCTCCATCTCCCTGGTGA
CTGTGAGCTGGGGGGGCCCCAACGGTGCTGGGGGGACCACGCTGGCTGTG
TTGCACCCAGAACGTGGCATCCGGCAATGGGCCCCTGCTCGCCAGGCCCG
CTGGGAAACCCAGAGCAGCATCTCTCTCATCCTGGAAGGCTCTGGGGCCA
GCAGCCCCTGCGCCAACACCACCTTCTGCTGCAAGTTTGCGTCCTTCCCT
GAGGGCTCCTGGGAGGCCTGTGGGAGCCTCCCGCCCAGCTCAGACCCAGG
GCTCTCTGCCCCGCCGACTCCTGCCCCCATTCTGCGGGCAGACCTGATTC
CGTGGCTCGGCCACCTCCTCGTGGGCCTCAGCGGGGCTTTTGGCTTCATC
ATCTTAGTGTACTTGCTGATCAACTGCAGGAACACCGGGCCATGGCTGAA
GAAGGTCCTGAAGTGTAACACCCCAGACCCCTCGAAGTTCTTTTCCCAGC
TGAGCTCAGAGCATGGAGGAGACGTCCAGAAGTGGCTCTCTTCGCCCTTC
CCCTCATCGTCCTTCAGCCCTGGCGGCCTGGCACCTGAGATCTCGCCACT
AGAAGTGCTGGAGAGGGACAAGGTGACGCAGCTGCTCCTGCAGCAGGACA
AGGTGCCTGAGCCCGCATCCTTAAGCAGCAACCACTCGCTGACCAGCTGC
TTCACCAACCAGGGTTACTTCTTCTTCCACCTCCCGGATGCCTTGGAGAT
AGAGGCCTGCCAGGTGTACTTTACTTACGACCCCTACTCAGAGGAAGACC
CTGATGAGGGTGTGGCCGGGGCACCCACAGGGTCTTCCCCCCAACCCCTG
CAGCCTCTGTCAGGGGAGGACGACGCCTACTGCACCTTCCCCTCCAGGGA
TGACCTGCTGCTCTTCTCCCCCAGTCTCCTCGGTGGCCCCAGCCCCCCAA
GCACTGCCCCTGGGGGCAGTGGGGCCGGTGAAGAGAGGATGCCCCCTTCT
TTGCAAGAAAGAGTCCCCAGAGACTGGGACCCCCAGCCCCTGGGGCCTCC
CACCCCAGGAGTCCCAGACCTGGTGGATTTTCAGCCACCCCCTGAGCTGG
TGCTGCGAGAGGCTGGGGAGGAGGTCCCTGACGCTGGCCCCAGGGAGGGA
GTCAGTTTCCCCTGGTCCAGGCCTCCTGGGCAGGGGGAGTTCAGGGCCCT
TAATGCTCGCCTGCCCCTGAACACTGATGCCTACTTGTCCCTCCAAGAAC
TCCAGGGTCAGGACCCAACTCACTTGGTGTAG.

Therefore, in some embodiments, the chimeric receptor is a CD112R-IL21R fusion and has the amino acid sequence:

(SEQ ID NO: 19)
TPEVWVQVRMEATELSSFTIRCGFLGSGSISLVTVSWGGPNGAGGTTLAV
LHPERGIRQWAPARQARWETQSSISLILEGSGASSPCANTTFCCKFASFP
EGSWEACGSLPPSSDPGLSAPPTPAPILRADLGWNPHLLLLLLLVIVFIP
AFWSLKTHPLWRLWKKIWAVPSPERFFMPLYKGCSGDFKKWVGAPFTGSS
LELGPWSPEVPSTLEVYSCHPPRSPAKRLQLTELQEPAELVESDGVPKPS
FWPTAQNSGGSAYSEERDRPYGLVSIDTVTVLDAEGPCTWPCSCEDDGYP
ALDLDAGLEPSPGLEDPLLDAGTTVLSCGCVSAGSPGLGGPLGSLLDRLK
PPLADGEDWAGGLPWGGRSPGGVSESEAGSPLAGLDMDTFDSGFVGSDCS
SPVECDFTSPGDEGPPRSYLRQWVVIPPPLSSPGPQAS.

Therefore, in some embodiments, the chimeric receptor is encoded by the

(SEQ ID NO: 51)
ACCCCGGAGGTGTGGGTTCAAGTTCGGATGGAGGCCACCGAGCTCTCGTC
CTTCACCATCCGTTGTGGGTTCCTGGGGTCTGGCTCCATCTCCCTGGTGA
CTGTGAGCTGGGGGGGCCCCAACGGTGCTGGGGGGACCACGCTGGCTGTG
TTGCACCCAGAACGTGGCATCCGGCAATGGGCCCCTGCTCGCCAGGCCCG
CTGGGAAACCCAGAGCAGCATCTCTCTCATCCTGGAAGGCTCTGGGGCCA
GCAGCCCCTGCGCCAACACCACCTTCTGCTGCAAGTTTGCGTCCTTCCCT
GAGGGCTCCTGGGAGGCCTGTGGGAGCCTCCCGCCCAGCTCAGACCCAGG
GCTCTCTGCCCCGCCGACTCCTGCCCCCATTCTGCGGGCAGACCTGGGCT
GGAACCCTCACCTGCTGCTTCTCCTCCTGCTTGTCATAGTCTTCATTCCT
GCCTTCTGGAGCCTGAAGACCCATCCATTGTGGAGGCTATGGAAGAAGAT
ATGGGCCGTCCCCAGCCCTGAGCGGTTCTTCATGCCCCTGTACAAGGGCT
GCAGCGGAGACTTCAAGAAATGGGTGGGTGCACCCTTCACTGGCTCCAGC
CTGGAGCTGGGACCCTGGAGCCCAGAGGTGCCCTCCACCCTGGAGGTGTA
CAGCTGCCACCCACCACGGAGCCCGGCCAAGAGGCTGCAGCTCACGGAGC
TACAAGAACCAGCAGAGCTGGTGGAGTCTGACGGTGTGCCCAAGCCCAGC
TTCTGGCCGACAGCCCAGAACTCGGGGGGCTCAGCTTACAGTGAGGAGAG
GGATCGGCCATACGGCCTGGTGTCCATTGACACAGTGACTGTGCTAGATG
CAGAGGGGCCATGCACCTGGCCCTGCAGCTGTGAGGATGACGGCTACCCA
GCCCTGGACCTGGATGCTGGCCTGGAGCCCAGCCCAGGCCTAGAGGACCC
ACTCTTGGATGCAGGGACCACAGTCCTGTCCTGTGGCTGTGTCTCAGCTG
GCAGCCCTGGGCTAGGAGGGCCCCTGGGAAGCCTCCTGGACAGACTAAAG
CCACCCCTTGCAGATGGGGAGGACTGGGCTGGGGGACTGCCCTGGGGTGG
CCGGTCACCTGGAGGGGTCTCAGAGAGTGAGGCGGGCTCACCCCTGGCCG
GCCTGGATATGGACACGTTTGACAGTGGCTTTGTGGGCTCTGACTGCAGC
AGCCCTGTGGAGTGTGACTTCACCAGCCCCGGGGACGAAGGACCCCCCCG
GAGCTACCTCCGCCAGTGGGTGGTCATTCCTCCGCCACTTTCGAGCCCTG
GACCCCAGGCCAGCTAATGA.

Therefore, in some embodiments, the chimeric receptor is a CD112R-IL12Rb2 fusion and has the amino acid sequence:

(SEQ ID NO: 20)
TPEVWVQVRMEATELSSFTIRCGFLGSGSISLVTVSWGGPNGAGGTTLAV
LHPERGIRQWAPARQARWETQSSISLILEGSGASSPCANTTFCCKFASFP
EGSWEACGSLPPSSDPGLSAPPTPAPILRADLWMAFVAPSICIAIIMVGI
FSTHYFQQKVFVLLAALRPQWCSREIPDPANSTCAKKYPIAEEKTQLPLD
RLLIDWPTPEDPEPLVISEVLHQVTPVFRHPPCSNWPQREKGIQGHQASE
KDMMHSASSPPPPRALQAESRQLVDLYKVLESRGSDPKPENPACPWTVLP
AGDLPTHDGYLPSNIDDLPSHEAPLADSLEELEPQHISLSVFPSSSLHPL
TFSCGDKLTLDQLKMRCDSLML.

Therefore, in some embodiments, the chimeric receptor is encoded by the

(SEQ ID NO: 52)
ACCCCGGAGGTGTGGGTTCAAGTTCGGATGGAGGCCACCGAGCTCTCGTC
CTTCACCATCCGTTGTGGGTTCCTGGGGTCTGGCTCCATCTCCCTGGTGA
CTGTGAGCTGGGGGGGCCCCAACGGTGCTGGGGGGACCACGCTGGCTGTG
TTGCACCCAGAACGTGGCATCCGGCAATGGGCCCCTGCTCGCCAGGCCCG
CTGGGAAACCCAGAGCAGCATCTCTCTCATCCTGGAAGGCTCTGGGGCCA
GCAGCCCCTGCGCCAACACCACCTTCTGCTGCAAGTTTGCGTCCTTCCCT
GAGGGCTCCTGGGAGGCCTGTGGGAGCCTCCCGCCCAGCTCAGACCCAGG
GCTCTCTGCCCCGCCGACTCCTGCCCCCATTCTGCGGGCAGACCTGTGGA
TGGCGTTTGTGGCACCAAGCATTTGCATTGCTATCATCATGGTGGGCATT
TTCTCAACGCATTACTTCCAGCAAAAGGTGTTTGTTCTCCTAGCAGCCCT
CAGACCTCAGTGGTGTAGCAGAGAAATTCCAGATCCAGCAAATAGCACTT
GCGCTAAGAAATATCCCATTGCAGAGGAGAAGACACAGCTGCCCTTGGAC
AGGCTCCTGATAGACTGGCCCACGCCTGAAGATCCTGAACCGCTGGTCAT
CAGTGAAGTCCTTCATCAAGTGACCCCAGTTTTCAGACATCCCCCCTGCT
CCAACTGGCCACAAAGGGAAAAAGGAATCCAAGGTCATCAGGCCTCTGAG
AAAGACATGATGCACAGTGCCTCAAGCCCACCACCTCCAAGAGCTCTCCA
AGCTGAGAGCAGACAACTGGTGGATCTGTACAAGGTGCTGGAGAGCAGGG
GCTCCGACCCAAAGCCAGAAAACCCAGCCTGTCCCTGGACGGTGCTCCCA
GCAGGTGACCTTCCCACCCATGATGGCTACTTACCCTCCAACATAGATGA
CCTCCCCTCACATGAGGCACCTCTCGCTGACTCTCTGGAAGAACTGGAGC
CTCAGCACATCTCCCTTTCTGTTTTCCCCTCAAGTTCTCTTCACCCACTC
ACCTTCTCCTGTGGTGATAAGCTGACTCTGGATCAGTTAAAGATGAGGTG
TGACTCCCTCATGCTCTGA.

Therefore, in some embodiments, the chimeric receptor is a CD112R-IL6ST fusion and has the amino acid sequence:

(SEQ ID NO: 21)
TPEVWVQVRMEATELSSFTIRCGFLGSGSISLVTVSWGGPNGAGGTTLAV
LHPERGIRQWAPARQARWETQSSISLILEGSGASSPCANTTFCCKFASFP
EGSWEACGSLPPSSDPGLSAPPTPAPILRADLAIVVPVCLAFLLTTLLGV
LFCFNKRDLIKKHIWPNVPDPSKSHIAQWSPHTPPRHNFNSKDQMYSDGN
FTDVSVVEIEANDKKPFPEDLKSLDLFKKEKINTEGHSSGIGGSSCMSSS
RPSISSSDENESSQNTSSTVQYSTVVHSGYRHQVPSVQVFSRSESTQPLL
DSEERPEDLQLVDHVDGGDGILPRQQYFKQNCSQHESSPDISHFERSKQV
SSVNEEDFVRLKQQISDHISQSCGSGQMKMFQEVSAADAFGPGTEGQVER
FETVGMEAATDEGMPKSYLPQTVRQGGYMPQ.

Therefore, in some embodiments, the chimeric receptor is encoded by the

(SEQ ID NO: 53)
ACCCCGGAGGTGTGGGTTCAAGTTCGGATGGAGGCCACCGAGCTCTCGTC
CTTCACCATCCGTTGTGGGTTCCTGGGGTCTGGCTCCATCTCCCTGGTGA
CTGTGAGCTGGGGGGGCCCCAACGGTGCTGGGGGGACCACGCTGGCTGTG
TTGCACCCAGAACGTGGCATCCGGCAATGGGCCCCTGCTCGCCAGGCCCG
CTGGGAAACCCAGAGCAGCATCTCTCTCATCCTGGAAGGCTCTGGGGCCA
GCAGCCCCTGCGCCAACACCACCTTCTGCTGCAAGTTTGCGTCCTTCCCT
GAGGGCTCCTGGGAGGCCTGTGGGAGCCTCCCGCCCAGCTCAGACCCAGG
GCTCTCTGCCCCGCCGACTCCTGCCCCCATTCTGCGGGCAGACCTGGCCA
TAGTCGTGCCTGTTTGCTTAGCATTCCTATTGACAACTCTTCTGGGAGTG
CTGTTCTGCTTTAATAAGCGAGACCTAATTAAAAAACACATCTGGCCTAA
TGTTCCAGATCCTTCAAAGAGTCATATTGCCCAGTGGTCACCTCACACTC
CTCCAAGGCACAATTTTAATTCAAAAGATCAAATGTATTCAGATGGCAAT
TTCACTGATGTAAGTGTTGTGGAAATAGAAGCAAATGACAAAAAGCCTTT
TCCAGAAGATCTGAAATCATTGGACCTGTTCAAAAAGGAAAAAATTAATA
CTGAAGGACACAGCAGTGGTATTGGGGGGTCTTCATGCATGTCATCTTCT
AGGCCAAGCATTTCTAGCAGTGATGAAAATGAATCTTCACAAAACACTTC
GAGCACTGTCCAGTATTCTACCGTGGTACACAGTGGCTACAGACACCAAG
TTCCGTCAGTCCAAGTCTTCTCAAGATCCGAGTCTACCCAGCCCTTGTTA
GATTCAGAGGAGCGGCCAGAAGATCTACAATTAGTAGATCATGTAGATGG
CGGTGATGGTATTTTGCCCAGGCAACAGTACTTCAAACAGAACTGCAGTC
AGCATGAATCCAGTCCAGATATTTCACATTTTGAAAGGTCAAAGCAAGTT
TCATCAGTCAATGAGGAAGATTTTGTTAGACTTAAACAGCAGATTTCAGA
TCATATTTCACAATCCTGTGGATCTGGGCAAATGAAAATGTTTCAGGAAG
TTTCTGCAGCAGATGCTTTTGGTCCAGGTACTGAGGGACAAGTAGAAAGA
TTTGAAACAGTTGGCATGGAGGCTGCGACTGATGAAGGCATGCCTAAAAG
TTACTTACCACAGACTGTACGGCAAGGCGGCTACATGCCTCAGT.

Therefore, in some embodiments, the chimeric receptor is a CD112R-CD27 fusion and has the amino acid sequence:

(SEQ ID NO: 22)
TPEVWVQVRMEATELSSFTIRCGFLGSGSISLVTVSWGGPNGAGGTTLA
VLHPERGIRQWAPARQARWETQSSISLILEGSGASSPCANTTFCCKFAS
FPEGSWEACGSLPPSSDPGLSAPPTPAPILRADLILVIFSGMFLVFTLA
GALFLHQRRKYRSNKGESPVEPAEPCRYSCPREEEGSTIPIQEDYRKPE
PACSP.

Therefore, in some embodiments, the chimeric receptor is encoded by the nucleic acid sequence:

(SEQ ID NO: 54)
ACCCCGGAGGTGTGGGTTCAAGTTCGGATGGAGGCCACCGAGCTCTCGT
CCTTCACCATCCGTTGTGGGTTCCTGGGGTCTGGCTCCATCTCCCTGGT
GACTGTGAGCTGGGGGGGCCCCAACGGTGCTGGGGGGACCACGCTGGCT
GTGTTGCACCCAGAACGTGGCATCCGGCAATGGGCCCCTGCTCGCCAGG
CCCGCTGGGAAACCCAGAGCAGCATCTCTCTCATCCTGGAAGGCTCTGG
GGCCAGCAGCCCCTGCGCCAACACCACCTTCTGCTGCAAGTTTGCGTCC
TTCCCTGAGGGCTCCTGGGAGGCCTGTGGGAGCCTCCCGCCCAGCTCAG
ACCCAGGGCTCTCTGCCCCGCCGACTCCTGCCCCCATTCTGCGGGCAGA
CCTGATCCTTGTGATCTTCTCTGGAATGTTCCTTGTTTTCACCCTGGCC
GGGGCCCTGTTCCTCCATCAACGAAGGAAATATAGATCAAACAAAGGAG
AAAGTCCTGTGGAGCCTGCAGAGCCTTGTCGTTACAGCTGCCCCAGGGA
GGAGGAGGGCAGCACCATCCCCATCCAGGAGGATTACCGAAAACCGGAG
CCTGCCTGCTCCCCCTGA.

Therefore, in some embodiments, the chimeric receptor is a CD112R-ICOS fusion and has the amino acid sequence:

(SEQ ID NO: 23)
TPEVWVQVRMEATELSSFTIRCGFLGSGSISLVTVSWGGPNGAGGTTLA
VLHPERGIRQWAPARQARWETQSSISLILEGSGASSPCANTTFCCKFAS
FPEGSWEACGSLPPSSDPGLSAPPTPAPILRADLFWLPIGCAAFVVVCI
LGCILICWLTKKKYSSSVHDPNGEYMFMRAVNTAKKSRLTDVTL.

Therefore, in some embodiments, the chimeric receptor is encoded by the nucleic acid sequence:

(SEQ ID NO: 55)
ACCCCGGAGGTGTGGGTTCAAGTTCGGATGGAGGCCACCGAGCTCTCGT
CCTTCACCATCCGTTGTGGGTTCCTGGGGTCTGGCTCCATCTCCCTGGT
GACTGTGAGCTGGGGGGGCCCCAACGGTGCTGGGGGGACCACGCTGGCT
GTGTTGCACCCAGAACGTGGCATCCGGCAATGGGCCCCTGCTCGCCAGG
CCCGCTGGGAAACCCAGAGCAGCATCTCTCTCATCCTGGAAGGCTCTGG
GGCCAGCAGCCCCTGCGCCAACACCACCTTCTGCTGCAAGTTTGCGTCC
TTCCCTGAGGGCTCCTGGGAGGCCTGTGGGAGCCTCCCGCCCAGCTCAG
ACCCAGGGCTCTCTGCCCCGCCGACTCCTGCCCCCATTCTGCGGGCAGA
CCTGTTCTGGTTACCCATAGGATGTGCAGCCTTTGTTGTAGTCTGCATT
TTGGGATGCATACTTATTTGTTGGCTTACAAAAAAGAAGTATTCATCCA
GTGTGCACGACCCTAACGGTGAATACATGTTCATGAGAGCAGTGAACAC
AGCCAAAAAATCTAGACTCACAGATGTGACCCTATAA.

Therefore, in some embodiments, the chimeric receptor is a CD112R-OX40 fusion and has the amino acid sequence:

(SEQ ID NO: 24)
TPEVWVQVRMEATELSSFTIRCGFLGSGSISLVTVSWGGPNGAGGTTLA
VLHPERGIRQWAPARQARWETQSSISLILEGSGASSPCANTTFCCKFAS
FPEGSWEACGSLPPSSDPGLSAPPTPAPILRADLVAAILGLGLVLGLLG
PLAILLALYLLRRDQRLPPDAHKPPGGGSFRTPIQEEQADAHSTLAKI.

Therefore, in some embodiments, the chimeric receptor is encoded by the

(SEQ ID NO: 56)
ACCCCGGAGGTGTGGGTTCAAGTTCGGATGGAGGCCACCGAGCTCTCGT
CCTTCACCATCCGTTGTGGGTTCCTGGGGTCTGGCTCCATCTCCCTGGT
GACTGTGAGCTGGGGGGGCCCCAACGGTGCTGGGGGGACCACGCTGGCT
GTGTTGCACCCAGAACGTGGCATCCGGCAATGGGCCCCTGCTCGCCAGG
CCCGCTGGGAAACCCAGAGCAGCATCTCTCTCATCCTGGAAGGCTCTGG
GGCCAGCAGCCCCTGCGCCAACACCACCTTCTGCTGCAAGTTTGCGTCC
TTCCCTGAGGGCTCCTGGGAGGCCTGTGGGAGCCTCCCGCCCAGCTCAG
ACCCAGGGCTCTCTGCCCCGCCGACTCCTGCCCCCATTCTGCGGGCAGA
CCTGGTTGCCGCCATCCTGGGCCTGGGCCTGGTGCTGGGGCTGCTGGGC
CCCCTGGCCATCCTGCTGGCCCTGTACCTGCTCCGGAGGGACCAGAGGC
TGCCCCCCGATGCCCACAAGCCCCCTGGGGGAGGCAGTTTCCGGACCCC
CATCCAAGAGGAGCAGGCCGACGCCCACTCCACCCTGGCCAAGATCTG
A.

Therefore, in some embodiments, the chimeric receptor is a DR5-IL7a fusion and has the amino acid sequence:

(SEQ ID NO: 25)
ITQQDLAPQQRAAPQQKRSSPSEGLCPPGHHISEDGRDCISCKYGQDYS
THWNDLLFCLRCTRCDSGEVELSPCTTTRNTVCQCEEGTFREEDSPEMC
RKCRTGCPRGMVKVGDCTPWSDIECVHKESGTKHSGEVPAVEETVTSSP
GTPASPCSPILLTISILSFFSVALLVILACVLWKKRIKPIVWPSLPDHK
KTLEHLCKKPRKNLNVSFNPESFLDCQIHRVDDIQARDEVEGFLQDTFP
QQLEESEKQRLGGDVQSPNCPSEDWVITPESFGRDSSLTCLAGNVSACD
APILSSSRSLDCRESGKNGPHVYQDLLLSLGTTNSTLPPPFSLQSGILT
LNPVAQGQPILTSLGSNQEEAYVTMSSFYQNQ.

Therefore, in some embodiments, the chimeric receptor is encoded by the nucleic acid sequence:

(SEQ ID NO: 57)
ATCACCCAACAAGACCTAGCTCCCCAGCAGAGAGCGGCCCCACAACAAA
AGAGGTCCAGCCCCTCAGAGGGATTGTGTCCACCTGGACACCATATCTC
AGAAGACGGTAGAGATTGCATCTCCTGCAAATATGGACAGGACTATAGC
ACTCACTGGAATGACCTCCTTTTCTGCTTGCGCTGCACCAGGTGTGATT
CAGGTGAAGTGGAGCTAAGTCCGTGCACCACGACCAGAAACACAGTGTG
TCAGTGCGAAGAAGGCACCTTCCGGGAAGAAGATTCTCCTGAGATGTGC
CGGAAGTGCCGCACAGGGTGTCCCAGAGGGATGGTCAAGGTCGGTGATT
GTACACCCTGGAGTGACATCGAATGTGTCCACAAAGAATCAGGTACAAA
GCACAGTGGGGAAGCCCCAGCTGTGGAGGAGACGGTGACCTCCAGCCCA
GGGACTCCTGCCTCTCCCTGTTCTCCTATCTTACTAACCATCAGCATTT
TGAGTTTTTTCTCTGTCGCTCTGTTGGTCATCTTGGCCTGTGTGTTATG
GAAAAAAAGGATTAAGCCTATCGTATGGCCCAGTCTCCCCGATCATAAG
AAGACTCTGGAACATCTTTGTAAGAAACCAAGAAAAAATTTAAATGTGA
GTTTCAATCCTGAAAGTTTCCTGGACTGCCAGATTCATAGGGTGGATGA
CATTCAAGCTAGAGATGAAGTGGAAGGTTTTCTGCAAGATACGTTTCCT
CAGCAACTAGAAGAATCTGAGAAGCAGAGGCTTGGAGGGGATGTGCAGA
GCCCCAACTGCCCATCTGAGGATGTAGTCGTCACTCCAGAAAGCTTTGG
AAGAGATTCATCCCTCACATGCCTGGCTGGGAATGTCAGTGCATGTGAC
GCCCCTATTCTCTCCTCTTCCAGGTCCCTAGACTGCAGGGAGAGTGGCA
AGAATGGGCCTCATGTGTACCAGGACCTCCTGCTTAGCCTTGGGACTAC
AAACAGCACGCTGCCCCCTCCATTTTCTCTCCAATCTGGAATCCTGACA
TTGAACCCAGTTGCTCAGGGTCAGCCCATTCTTACTTCCCTGGGATCAA
ATCAAGAAGAAGCATATGTCACCATGTCCAGCTTCTACCAAAACCAGTG
A.

Therefore, in some embodiments, the chimeric receptor is a DR5-β/IL2, 15 fusion and has the amino acid sequence:

(SEQ ID NO: 26)
ITQQDLAPQQRAAPQQKRSSPSEGLCPPGHHISEDGRDCISCKYGQDYS
THWNDLLFCLRCTRCDSGEVELSPCTTTRNTVCQCEEGTFREEDSPEMC
RKCRTGCPRGMVKVGDCTPWSDIECVHKESGTKHSGEVPAVEETVTSSP
GTPASPCSIPWLGHLLVGLSGAFGFIILVYLLINCRNTGPWLKKVLKCN
TPDPSKFFSQLSSEHGGDVQKWLSSPFPSSSFSPGGLAPEISPLEVLER
DKVTQLLLQQDKVPEPASLSSNHSLTSCFTNQGYFFFHLPDALEIEACQ
VYFTYDPYSEEDPDEGVAGAPTGSSPQPLQPLSGEDDAYCTFPSRDDLL
LFSPSLLGGPSPPSTAPGGSGAGEERMPPSLQERVPRDWDPQPLGPPTP
GVPDLVDFQPPPELVLREAGEEVPDAGPREGVSFPWSRPPGQGEFRALN
ARLPLNTDAYLSLQELQGQDPTHLV.

Therefore, in some embodiments, the chimeric receptor is encoded by the

(SEQ ID NO: 58)
ATCACCCAACAAGACCTAGCTCCCCAGCAGAGAGCGGCCCCACAACAAA
AGAGGTCCAGCCCCTCAGAGGGATTGTGTCCACCTGGACACCATATCTC
AGAAGACGGTAGAGATTGCATCTCCTGCAAATATGGACAGGACTATAGC
ACTCACTGGAATGACCTCCTTTTCTGCTTGCGCTGCACCAGGTGTGATT
CAGGTGAAGTGGAGCTAAGTCCGTGCACCACGACCAGAAACACAGTGTG
TCAGTGCGAAGAAGGCACCTTCCGGGAAGAAGATTCTCCTGAGATGTGC
CGGAAGTGCCGCACAGGGTGTCCCAGAGGGATGGTCAAGGTCGGTGATT
GTACACCCTGGAGTGACATCGAATGTGTCCACAAAGAATCAGGTACAAA
GCACAGTGGGGAAGCCCCAGCTGTGGAGGAGACGGTGACCTCCAGCCCA
GGGACTCCTGCCTCTCCCTGTTCTATTCCGTGGCTCGGCCACCTCCTCG
TGGGCCTCAGCGGGGCTTTTGGCTTCATCATCTTAGTGTACTTGCTGAT
CAACTGCAGGAACACCGGGCCATGGCTGAAGAAGGTCCTGAAGTGTAAC
ACCCCAGACCCCTCGAAGTTCTTTTCCCAGCTGAGCTCAGAGCATGGAG
GAGACGTCCAGAAGTGGCTCTCTTCGCCCTTCCCCTCATCGTCCTTCAG
CCCTGGCGGCCTGGCACCTGAGATCTCGCCACTAGAAGTGCTGGAGAGG
GACAAGGTGACGCAGCTGCTCCTGCAGCAGGACAAGGTGCCTGAGCCCG
CATCCTTAAGCAGCAACCACTCGCTGACCAGCTGCTTCACCAACCAGGG
TTACTTCTTCTTCCACCTCCCGGATGCCTTGGAGATAGAGGCCTGCCAG
GTGTACTTTACTTACGACCCCTACTCAGAGGAAGACCCTGATGAGGGTG
TGGCCGGGGCACCCACAGGGTCTTCCCCCCAACCCCTGCAGCCTCTGTC
AGGGGAGGACGACGCCTACTGCACCTTCCCCTCCAGGGATGACCTGCTG
CTCTTCTCCCCCAGTCTCCTCGGTGGCCCCAGCCCCCCAAGCACTGCCC
CTGGGGGCAGTGGGGCCGGTGAAGAGAGGATGCCCCCTTCTTTGCAAGA
AAGAGTCCCCAGAGACTGGGACCCCCAGCCCCTGGGGCCTCCCACCCCA
GGAGTCCCAGACCTGGTGGATTTTCAGCCACCCCCTGAGCTGGTGCTGC
GAGAGGCTGGGGAGGAGGTCCCTGACGCTGGCCCCAGGGAGGGAGTCAG
TTTCCCCTGGTCCAGGCCTCCTGGGCAGGGGGAGTTCAGGGCCCTTAAT
GCTCGCCTGCCCCTGAACACTGATGCCTACTTGTCCCTCCAAGAACTCC
AGGGTCAGGACCCAACTCACTTGGTGTAG.

Therefore, in some embodiments, the chimeric receptor is a DR5-IL21R fusion and has the amino acid sequence:

(SEQ ID NO: 27)
ITQQDLAPQQRAAPQQKRSSPSEGLCPPGHHISEDGRDCISCKYGQDYS
THWNDLLFCLRCTRCDSGEVELSPCTTTRNTVCQCEEGTFREEDSPEMC
RKCRTGCPRGMVKVGDCTPWSDIECVHKESGTKHSGEVPAVEETVTSSP
GTPASPCSGWNPHLLLLLLLVIVFIPAFWSLKTHPLWRLWKKIWAVPSP
ERFFMPLYKGCSGDFKKWVGAPFTGSSLELGPWSPEVPSTLEVYSCHPP
RSPAKRLQLTELQEPAELVESDGVPKPSFWPTAQNSGGSAYSEERDRPY
GLVSIDTVTVLDAEGPCTWPCSCEDDGYPALDLDAGLEPSPGLEDPLLD
AGTTVLSCGCVSAGSPGLGGPLGSLLDRLKPPLADGEDWAGGLPWGGRS
PGGVSESEAGSPLAGLDMDTFDSGFVGSDCSSPVECDFTSPGDEGPPRS
YLRQWVVIPPPLSSPGPQAS.

Therefore, in some embodiments, the chimeric receptor is encoded by the

(SEQ ID NO: 59)
ATCACCCAACAAGACCTAGCTCCCCAGCAGAGAGCGGCCCCACAACAAA
AGAGGTCCAGCCCCTCAGAGGGATTGTGTCCACCTGGACACCATATCTC
AGAAGACGGTAGAGATTGCATCTCCTGCAAATATGGACAGGACTATAGC
ACTCACTGGAATGACCTCCTTTTCTGCTTGCGCTGCACCAGGTGTGATT
CAGGTGAAGTGGAGCTAAGTCCGTGCACCACGACCAGAAACACAGTGTG
TCAGTGCGAAGAAGGCACCTTCCGGGAAGAAGATTCTCCTGAGATGTGC
CGGAAGTGCCGCACAGGGTGTCCCAGAGGGATGGTCAAGGTCGGTGATT
GTACACCCTGGAGTGACATCGAATGTGTCCACAAAGAATCAGGTACAAA
GCACAGTGGGGAAGCCCCAGCTGTGGAGGAGACGGTGACCTCCAGCCCA
GGGACTCCTGCCTCTCCCTGTTCTGGCTGGAACCCTCACCTGCTGCTTC
TCCTCCTGCTTGTCATAGTCTTCATTCCTGCCTTCTGGAGCCTGAAGAC
CCATCCATTGTGGAGGCTATGGAAGAAGATATGGGCCGTCCCCAGCCCT
GAGCGGTTCTTCATGCCCCTGTACAAGGGCTGCAGCGGAGACTTCAAGA
AATGGGTGGGTGCACCCTTCACTGGCTCCAGCCTGGAGCTGGGACCCTG
GAGCCCAGAGGTGCCCTCCACCCTGGAGGTGTACAGCTGCCACCCACCA
CGGAGCCCGGCCAAGAGGCTGCAGCTCACGGAGCTACAAGAACCAGCAG
AGCTGGTGGAGTCTGACGGTGTGCCCAAGCCCAGCTTCTGGCCGACAGC
CCAGAACTCGGGGGGCTCAGCTTACAGTGAGGAGAGGGATCGGCCATAC
GGCCTGGTGTCCATTGACACAGTGACTGTGCTAGATGCAGAGGGGCCAT
GCACCTGGCCCTGCAGCTGTGAGGATGACGGCTACCCAGCCCTGGACCT
GGATGCTGGCCTGGAGCCCAGCCCAGGCCTAGAGGACCCACTCTTGGAT
GCAGGGACCACAGTCCTGTCCTGTGGCTGTGTCTCAGCTGGCAGCCCTG
GGCTAGGAGGGCCCCTGGGAAGCCTCCTGGACAGACTAAAGCCACCCCT
TGCAGATGGGGAGGACTGGGCTGGGGGACTGCCCTGGGGTGGCCGGTCA
CCTGGAGGGGTCTCAGAGAGTGAGGCGGGCTCACCCCTGGCCGGCCTGG
ATATGGACACGTTTGACAGTGGCTTTGTGGGCTCTGACTGCAGCAGCCC
TGTGGAGTGTGACTTCACCAGCCCCGGGGACGAAGGACCCCCCCGGAGC
TACCTCCGCCAGTGGGTGGTCATTCCTCCGCCACTTTCGAGCCCTGGAC
CCCAGGCCAGCTAATGA.

Therefore, in some embodiments, the chimeric receptor is a DR5-IL12Rb2 fusion and has the amino acid sequence:

(SEQ ID NO: 28)
ITQQDLAPQQRAAPQQKRSSPSEGLCPPGHHISEDGRDCISCKYGQDYS
THWNDLLFCLRCTRCDSGEVELSPCTTTRNTVCQCEEGTFREEDSPEMC
RKCRTGCPRGMVKVGDCTPWSDIECVHKESGTKHSGEVPAVEETVTSSP
GTPASPCSWMAFVAPSICIAIIMVGIFSTHYFQQKVFVLLAALRPQWCS
REIPDPANSTCAKKYPIAEEKTQLPLDRLLIDWPTPEDPEPLVISEVLH
QVTPVFRHPPCSNWPQREKGIQGHQASEKDMMHSASSPPPPRALQAESR
QLVDLYKVLESRGSDPKPENPACPWTVLPAGDLPTHDGYLPSNIDDLPS
HEAPLADSLEELEPQHISLSVFPSSSLHPLTFSCGDKLTLDQLKMRCDS
LML.

Therefore, in some embodiments, the chimeric receptor is encoded by the

(SEQ ID NO: 60)
ATCACCCAACAAGACCTAGCTCCCCAGCAGAGAGCGGCCCCACAACAAA
AGAGGTCCAGCCCCTCAGAGGGATTGTGTCCACCTGGACACCATATCTC
AGAAGACGGTAGAGATTGCATCTCCTGCAAATATGGACAGGACTATAGC
ACTCACTGGAATGACCTCCTTTTCTGCTTGCGCTGCACCAGGTGTGATT
CAGGTGAAGTGGAGCTAAGTCCGTGCACCACGACCAGAAACACAGTGTG
TCAGTGCGAAGAAGGCACCTTCCGGGAAGAAGATTCTCCTGAGATGTGC
CGGAAGTGCCGCACAGGGTGTCCCAGAGGGATGGTCAAGGTCGGTGATT
GTACACCCTGGAGTGACATCGAATGTGTCCACAAAGAATCAGGTACAAA
GCACAGTGGGGAAGCCCCAGCTGTGGAGGAGACGGTGACCTCCAGCCCA
GGGACTCCTGCCTCTCCCTGTTCTTGGATGGCGTTTGTGGCACCAAGCA
TTTGCATTGCTATCATCATGGTGGGCATTTTCTCAACGCATTACTTCCA
GCAAAAGGTGTTTGTTCTCCTAGCAGCCCTCAGACCTCAGTGGTGTAGC
AGAGAAATTCCAGATCCAGCAAATAGCACTTGCGCTAAGAAATATCCCA
TTGCAGAGGAGAAGACACAGCTGCCCTTGGACAGGCTCCTGATAGACTG
GCCCACGCCTGAAGATCCTGAACCGCTGGTCATCAGTGAAGTCCTTCAT
CAAGTGACCCCAGTTTTCAGACATCCCCCCTGCTCCAACTGGCCACAAA
GGGAAAAAGGAATCCAAGGTCATCAGGCCTCTGAGAAAGACATGATGCA
CAGTGCCTCAAGCCCACCACCTCCAAGAGCTCTCCAAGCTGAGAGCAGA
CAACTGGTGGATCTGTACAAGGTGCTGGAGAGCAGGGGCTCCGACCCAA
AGCCAGAAAACCCAGCCTGTCCCTGGACGGTGCTCCCAGCAGGTGACCT
TCCCACCCATGATGGCTACTTACCCTCCAACATAGATGACCTCCCCTCA
CATGAGGCACCTCTCGCTGACTCTCTGGAAGAACTGGAGCCTCAGCACA
TCTCCCTTTCTGTTTTCCCCTCAAGTTCTCTTCACCCACTCACCTTCTC
CTGTGGTGATAAGCTGACTCTGGATCAGTTAAAGATGAGGTGTGACTCC
CTCATGCTCTGA.

Therefore, in some embodiments, the chimeric receptor is a DR5-IL6ST fusion and has the amino acid sequence:

(SEQ ID NO: 29)
ITQQDLAPQQRAAPQQKRSSPSEGLCPPGHHISEDGRDCISCKYGQDYS
THWNDLLFCLRCTRCDSGEVELSPCTTTRNTVCQCEEGTFREEDSPEMC
RKCRTGCPRGMVKVGDCTPWSDIECVHKESGTKHSGEVPAVEETVTSSP
GTPASPCSAIVVPVCLAFLLTTLLGVLFCFNKRDLIKKHIWPNVPDPSK
SHIAQWSPHTPPRHNFNSKDQMYSDGNFTDVSVVEIEANDKKPFPEDLK
SLDLFKKEKINTEGHSSGIGGSSCMSSSRPSISSSDENESSQNTSSTVQ
YSTWVHSGYRHQVPSVQVFSRSESTQPLLDSEERPEDLQLVDHVDGGDG
ILPRQQYFKQNCSQHESSPDISHFERSKQVSSVNEEDFVRLKQQISDHI
SQSCGSGQMKMFQEVSAADAFGPGTEGQVERFETVGMEAATDEGMPKSY
LPQTVRQGGYMPQ.

Therefore, in some embodiments, the chimeric receptor is encoded by the

(SEQ ID NO: 61)
ATCACCCAACAAGACCTAGCTCCCCAGCAGAGAGCGGCCCCACAACAAA
AGAGGTCCAGCCCCTCAGAGGGATTGTGTCCACCTGGACACCATATCTC
AGAAGACGGTAGAGATTGCATCTCCTGCAAATATGGACAGGACTATAGC
ACTCACTGGAATGACCTCCTTTTCTGCTTGCGCTGCACCAGGTGTGATT
CAGGTGAAGTGGAGCTAAGTCCGTGCACCACGACCAGAAACACAGTGTG
TCAGTGCGAAGAAGGCACCTTCCGGGAAGAAGATTCTCCTGAGATGTGC
CGGAAGTGCCGCACAGGGTGTCCCAGAGGGATGGTCAAGGTCGGTGATT
GTACACCCTGGAGTGACATCGAATGTGTCCACAAAGAATCAGGTACAAA
GCACAGTGGGGAAGCCCCAGCTGTGGAGGAGACGGTGACCTCCAGCCCA
GGGACTCCTGCCTCTCCCTGTTCTGCCATAGTCGTGCCTGTTTGCTTAG
CATTCCTATTGACAACTCTTCTGGGAGTGCTGTTCTGCTTTAATAAGCG
AGACCTAATTAAAAAACACATCTGGCCTAATGTTCCAGATCCTTCAAAG
AGTCATATTGCCCAGTGGTCACCTCACACTCCTCCAAGGCACAATTTTA
ATTCAAAAGATCAAATGTATTCAGATGGCAATTTCACTGATGTAAGTGT
TGTGGAAATAGAAGCAAATGACAAAAAGCCTTTTCCAGAAGATCTGAAA
TCATTGGACCTGTTCAAAAAGGAAAAAATTAATACTGAAGGACACAGCA
GTGGTATTGGGGGGTCTTCATGCATGTCATCTTCTAGGCCAAGCATTTC
TAGCAGTGATGAAAATGAATCTTCACAAAACACTTCGAGCACTGTCCAG
TATTCTACCGTGGTACACAGTGGCTACAGACACCAAGTTCCGTCAGTCC
AAGTCTTCTCAAGATCCGAGTCTACCCAGCCCTTGTTAGATTCAGAGGA
GCGGCCAGAAGATCTACAATTAGTAGATCATGTAGATGGCGGTGATGGT
ATTTTGCCCAGGCAACAGTACTTCAAACAGAACTGCAGTCAGCATGAAT
CCAGTCCAGATATTTCACATTTTGAAAGGTCAAAGCAAGTTTCATCAGT
CAATGAGGAAGATTTTGTTAGACTTAAACAGCAGATTTCAGATCATATT
TCACAATCCTGTGGATCTGGGCAAATGAAAATGTTTCAGGAAGTTTCTG
CAGCAGATGCTTTTGGTCCAGGTACTGAGGGACAAGTAGAAAGATTTGA
AACAGTTGGCATGGAGGCTGCGACTGATGAAGGCATGCCTAAAAGTTAC
TTACCACAGACTGTACGGCAAGGCGGCTACATGCCTCAGT.

Therefore, in some embodiments, the chimeric receptor is a DR5-CD27 fusion and has the amino acid sequence

(SEQ ID NO: 30)
ITQQDLAPQQRAAPQQKRSSPSEGLCPPGHHISEDGRDCISCKYGQDYS
THWNDLLFCLRCTRCDSGEVELSPCTTTRNTVCQCEEGTFREEDSPEMC
RKCRTGCPRGMVKVGDCTPWSDIECVHKESGTKHSGEVPAVEETVTSSP
GTPASPCSILVIFSGMFLVFTLAGALFLHQRRKYRSNKGESPVEPAEPC
RYSCPREEEGSTIPIQEDYRKPEPACSP.

Therefore, in some embodiments, the chimeric receptor is encoded by the

(SEQ ID NO: 62)
ATCACCCAACAAGACCTAGCTCCCCAGCAGAGAGCGGCCCCACAACAAA
AGAGGTCCAGCCCCTCAGAGGGATTGTGTCCACCTGGACACCATATCTC
AGAAGACGGTAGAGATTGCATCTCCTGCAAATATGGACAGGACTATAGC
ACTCACTGGAATGACCTCCTTTTCTGCTTGCGCTGCACCAGGTGTGATT
CAGGTGAAGTGGAGCTAAGTCCGTGCACCACGACCAGAAACACAGTGTG
TCAGTGCGAAGAAGGCACCTTCCGGGAAGAAGATTCTCCTGAGATGTGC
CGGAAGTGCCGCACAGGGTGTCCCAGAGGGATGGTCAAGGTCGGTGATT
GTACACCCTGGAGTGACATCGAATGTGTCCACAAAGAATCAGGTACAAA
GCACAGTGGGGAAGCCCCAGCTGTGGAGGAGACGGTGACCTCCAGCCCA
GGGACTCCTGCCTCTCCCTGTTCTATCCTTGTGATCTTCTCTGGAATGT
TCCTTGTTTTCACCCTGGCCGGGGCCCTGTTCCTCCATCAACGAAGGAA
ATATAGATCAAACAAAGGAGAAAGTCCTGTGGAGCCTGCAGAGCCTTGT
CGTTACAGCTGCCCCAGGGAGGAGGAGGGCAGCACCATCCCCATCCAGG
AGGATTACCGAAAACCGGAGCCTGCCTGCTCCCCCTGA.

Therefore, in some embodiments, the chimeric receptor is a DR5-ICOS fusion and has the amino acid sequence:

(SEQ ID NO: 31)
ITQQDLAPQQRAAPQQKRSSPSEGLCPPGHHISEDGRDCISCKYGQDYS
THWNDLLFCLRCTRCDSGEVELSPCTTTRNTVCQCEEGTFREEDSPEMC
RKCRTGCPRGMVKVGDCTPWSDIECVHKESGTKHSGEVPAVEETVTSSP
GTPASPCSFWLPIGCAAFVVVCILGCILICWLTKKKYSSSVHDPNGEYM
FMRAVNTAKKSRLTDVTL.

Therefore, in some embodiments, the chimeric receptor is encoded by the nucleic acid sequence:

(SEQ ID NO: 63)
ATCACCCAACAAGACCTAGCTCCCCAGCAGAGAGCGGCCCCACAACAAA
AGAGGTCCAGCCCCTCAGAGGGATTGTGTCCACCTGGACACCATATCTC
AGAAGACGGTAGAGATTGCATCTCCTGCAAATATGGACAGGACTATAGC
ACTCACTGGAATGACCTCCTTTTCTGCTTGCGCTGCACCAGGTGTGATT
CAGGTGAAGTGGAGCTAAGTCCGTGCACCACGACCAGAAACACAGTGTG
TCAGTGCGAAGAAGGCACCTTCCGGGAAGAAGATTCTCCTGAGATGTGC
CGGAAGTGCCGCACAGGGTGTCCCAGAGGGATGGTCAAGGTCGGTGATT
GTACACCCTGGAGTGACATCGAATGTGTCCACAAAGAATCAGGTACAAA
GCACAGTGGGGAAGCCCCAGCTGTGGAGGAGACGGTGACCTCCAGCCCA
GGGACTCCTGCCTCTCCCTGTTCTTTCTGGTTACCCATAGGATGTGCAG
CCTTTGTTGTAGTCTGCATTTTGGGATGCATACTTATTTGTTGGCTTAC
AAAAAAGAAGTATTCATCCAGTGTGCACGACCCTAACGGTGAATACATG
TTCATGAGAGCAGTGAACACAGCCAAAAAATCTAGACTCACAGATGTGA
CCCTATAA.

Therefore, in some embodiments, the chimeric receptor is a DR5-OX40 fusion and has the amino acid sequence:

(SEQ ID NO: 32)
ITQQDLAPQQRAAPQQKRSSPSEGLCPPGHHISEDGRDCISCKYGQDYS
THWNDLLFCLRCTRCDSGEVELSPCTTTRNTVCQCEEGTFREEDSPEMC
RKCRTGCPRGMVKVGDCTPWSDIECVHKESGTKHSGEVPAVEETVTSSP
GTPASPCSVAAILGLGLVLGLLGPLAILLALYLLRRDQRLPPDAHKPPG
GGSFRTPIQEEQADAHSTLAKI.

Therefore, in some embodiments, the chimeric receptor is encoded by the

(SEQ ID NO: 64)
ATCACCCAACAAGACCTAGCTCCCCAGCAGAGAGCGGCCCCACAACAAA
AGAGGTCCAGCCCCTCAGAGGGATTGTGTCCACCTGGACACCATATCTC
AGAAGACGGTAGAGATTGCATCTCCTGCAAATATGGACAGGACTATAGC
ACTCACTGGAATGACCTCCTTTTCTGCTTGCGCTGCACCAGGTGTGATT
CAGGTGAAGTGGAGCTAAGTCCGTGCACCACGACCAGAAACACAGTGTG
TCAGTGCGAAGAAGGCACCTTCCGGGAAGAAGATTCTCCTGAGATGTGC
CGGAAGTGCCGCACAGGGTGTCCCAGAGGGATGGTCAAGGTCGGTGATT
GTACACCCTGGAGTGACATCGAATGTGTCCACAAAGAATCAGGTACAAA
GCACAGTGGGGAAGCCCCAGCTGTGGAGGAGACGGTGACCTCCAGCCCA
GGGACTCCTGCCTCTCCCTGTTCTGTTGCCGCCATCCTGGGCCTGGGCC
TGGTGCTGGGGCTGCTGGGCCCCCTGGCCATCCTGCTGGCCCTGTACCT
GCTCCGGAGGGACCAGAGGCTGCCCCCCGATGCCCACAAGCCCCCTGGG
GGAGGCAGTTTCCGGACCCCCATCCAAGAGGAGCAGGCCGACGCCCACT
CCACCCTGGCCAAGATC.

In some embodiments, the nucleic acid encoding the disclosed chimeric receptors, CAR polypeptides, and/or shRNA that allow expression of the chimeric receptors, CAR polypeptides, and/or shRNA further encodes a P2A and/or CD8a signaling peptide.

P2A (2A peptides) are 18-22 amino-acid (aa)-long viral oligopeptides that mediate “self-cleavage” of polypeptides during translation in eukaryotic cells.

In some embodiments, the P2A has the amino acid sequence: GSGATNFSLLKQAGDVEENPGP (SEQ ID NO: 100). In some embodiments, the P2A is encoded by the nucleic acid sequence:

(SEQ ID NO: 101)
GGCAGCGGCGCCACCAACTTCAGCCTGCTGAAGCAGGCCGGCGACGTGG
AGGAGAACCCCGGCCCC.

In some embodiments, the CD8a signaling peptide has the amino acid sequence: MALPVTALLLPLALLLHAARP (SEQ ID NO: 102). In some embodiments, the CD8a signaling peptide is encoded by the nucleic acid sequence:

(SEQ ID NO: 103)
ATGGCCTTACCAGTGACCGCCTTGCTCCTGCCGCTGGCCTTGCTGCTCC
ACGCCGCCAGGCCG.

Therefore, in some embodiments, the chimeric receptor is a P2A-CD8-Tim3-IL7Rα fusion and has the amino acid sequence:

(SEQ ID NO: 104)
GSGATNFSLLKQAGDVEENPGPMALPVTALLLPLALLLHAARPSEVEYR
AEVGQNAYLPCFYTPAAPGNLVPVCWGKGACPVFECGNVVLRTDERDVN
YWTSRYWLNGDFRKGDVSLTIENVTLADSGIYCCRIQIPGIMNDEKFNL
KLVIKPAKVTPAPTLQRDFTAAFPRMLTTRGHGPAETQTLGSLPDINLT
QISTLANELRDSRLANDLRDSGATIRIGPILLTISILSFFSVALLVILA
CVLWKKRIKPIVWPSLPDHKKTLEHLCKKPRKNLNVSFNPESFLDCQIH
RVDDIQARDEVEGFLQDTFPQQLEESEKQRLGGDVQSPNCPSEDVVITP
ESFGRDSSLTCLAGNVSACDAPILSSSRSLDCRESGKNGPHVYQDLLLS
LGTTNSTLPPPFSLQSGILTLNPVAQGQPILTSLGSNQEEAYVTMSSFY
QNQ.

Therefore, in some embodiments, the chimeric receptor is encoded by the nucleic acid sequence:

(SEQ ID NO: 105)
GGCAGCGGCGCCACCAACTTCAGCCTGCTGAAGCAGGCCGGCGACGTGG
AGGAGAACCCCGGCCCCATGGCCTTACCAGTGACCGCCTTGCTCCTGCC
GCTGGCCTTGCTGCTCCACGCCGCCAGGCCGTCAGAAGTGGAATACAGA
GCGGAGGTCGGTCAGAATGCCTATCTGCCCTGCTTCTACACCCCAGCCG
CCCCAGGGAACCTCGTGCCCGTCTGCTGGGGCAAAGGAGCCTGTCCTGT
GTTTGAATGTGGCAACGTGGTGCTCAGGACTGATGAAAGGGATGTGAAT
TATTGGACATCCAGATACTGGCTAAATGGGGATTTCCGCAAAGGAGATG
TGTCCCTGACCATAGAGAATGTGACTCTAGCAGACAGTGGGATCTACTG
CTGCCGGATCCAAATCCCAGGCATAATGAATGATGAAAAATTTAACCTG
AAGTTGGTCATCAAACCAGCCAAGGTCACCCCTGCACCGACTCTGCAGA
GAGACTTCACTGCAGCCTTTCCAAGGATGCTTACCACCAGGGGACATGG
CCCAGCAGAGACACAGACACTGGGGAGCCTCCCTGATATAAATCTAACA
CAAATATCCACATTGGCCAATGAGTTACGGGACTCTAGATTGGCCAATG
ACTTACGGGACTCTGGAGCAACCATCAGAATAGGCCCTATCTTACTAAC
CATCAGCATTTTGAGTTTTTTCTCTGTCGCTCTGTTGGTCATCTTGGCC
TGTGTGTTATGGAAAAAAAGGATTAAGCCTATCGTATGGCCCAGTCTCC
CCGATCATAAGAAGACTCTGGAACATCTTTGTAAGAAACCAAGAAAAAA
TTTAAATGTGAGTTTCAATCCTGAAAGTTTCCTGGACTGCCAGATTCAT
AGGGTGGATGACATTCAAGCTAGAGATGAAGTGGAAGGTTTTCTGCAAG
ATACGTTTCCTCAGCAACTAGAAGAATCTGAGAAGCAGAGGCTTGGAGG
GGATGTGCAGAGCCCCAACTGCCCATCTGAGGATGTAGTCGTCACTCCA
GAAAGCTTTGGAAGAGATTCATCCCTCACATGCCTGGCTGGGAATGTCA
GTGCATGTGACGCCCCTATTCTCTCCTCTTCCAGGTCCCTAGACTGCAG
GGAGAGTGGCAAGAATGGGCCTCATGTGTACCAGGACCTCCTGCTTAGC
CTTGGGACTACAAACAGCACGCTGCCCCCTCCATTTTCTCTCCAATCTG
GAATCCTGACATTGAACCCAGTTGCTCAGGGTCAGCCCATTCTTACTTC
CCTGGGATCAAATCAAGAAGAAGCATATGTCACCATGTCCAGCTTCTAC
CAAAACCAG.

Therefore, in some embodiments, the chimeric receptor is a P2A-CD8-LAG3-OX40 fusion and has the amino acid sequence:

(SEQ ID NO: 106)
GSGATNFSLLKQAGDVEENPGPMALPVTALLLPLALLLHAARPLQPGAE
VPVVWAQEGAPAQLPCSPTIPLQDLSLLRRAGVTWQHQPDSGPPAAAPG
HPLAPGPHPAAPSSWGPRPRRYTVLSVGPGGLRSGRLPLQPRVQLDERG
RQRGDFSLWLRPARRADAGEYRAAVHLRDRALSCRLRLRLGQASMTASP
PGSLRASDWVILNCSFSRPDRPASVHWFRNRGQGRVPVRESPHHHLAES
FLFLPQVSPMDSGPWGCILTYRDGFNVSIMYNLTVLGLEPPTPLTVYAG
AGSRVGLPCRLPAGVGTRSFLTAKWTPPGGGPDLLVTGDNGDFTLRLED
VSQAQAGTYTCHIHLQEQQLNATVTLAIITVTPKSFGSPGSLGKLLCEV
TPVSGQERFVWSSLDTPSQRSFSGPWLEAQEAQLLSQPWQCQLYQGERL
LGAAVYFTELSSPGAQRSGRAPGALPAGHLVAAILGLGLVLGLLGPLAI
LLALYLLRRDQRLPPDAHKPPGGGSFRTPIQEEQADAHSTLAKI.

Therefore, in some embodiments, the chimeric receptor is encoded by the

(SEQ ID NO: 107)
GGCAGCGGCGCCACCAACTTCAGCCTGCTGAAGCAGGCCGGCGACGTGG
AGGAGAACCCCGGCCCCATGGCCTTACCAGTGACCGCCTTGCTCCTGCC
GCTGGCCTTGCTGCTCCACGCCGCCAGGCCGCTCCAGCCAGGGGCTGAG
GTCCCGGTGGTGTGGGCCCAGGAGGGGGCTCCTGCCCAGCTCCCCTGCA
GCCCCACAATCCCCCTCCAGGATCTCAGCCTTCTGCGAAGAGCAGGGGT
CACTTGGCAGCATCAGCCAGACAGTGGCCCGCCCGCTGCCGCCCCCGGC
CATCCCCTGGCCCCCGGCCCTCACCCGGCGGCGCCCTCCTCCTGGGGGC
CCAGGCCCCGCCGCTACACGGTGCTGAGCGTGGGTCCCGGAGGCCTGCG
CAGCGGGAGGCTGCCCCTGCAGCCCCGCGTCCAGCTGGATGAGCGCGGC
CGGCAGCGCGGGGACTTCTCGCTATGGCTGCGCCCAGCCCGGCGCGCGG
ACGCCGGCGAGTACCGCGCCGCGGTGCACCTCAGGGACCGCGCCCTCTC
CTGCCGCCTCCGTCTGCGCCTGGGCCAGGCCTCGATGACTGCCAGCCCC
CCAGGATCTCTCAGAGCCTCCGACTGGGTCATTTTGAACTGCTCCTTCA
GCCGCCCTGACCGCCCAGCCTCTGTGCATTGGTTCCGGAACCGGGGCCA
GGGCCGAGTCCCTGTCCGGGAGTCCCCCCATCACCACTTAGCGGAAAGC
TTCCTCTTCCTGCCCCAAGTCAGCCCCATGGACTCTGGGCCCTGGGGCT
GCATCCTCACCTACAGAGATGGCTTCAACGTCTCCATCATGTATAACCT
CACTGTTCTGGGTCTGGAGCCCCCAACTCCCTTGACAGTGTACGCTGGA
GCAGGTTCCAGGGTGGGGCTGCCCTGCCGCCTGCCTGCTGGTGTGGGGA
CCCGGTCTTTCCTCACTGCCAAGTGGACTCCTCCTGGGGGAGGCCCTGA
CCTCCTGGTGACTGGAGACAATGGCGACTTTACCCTTCGACTAGAGGAT
GTGAGCCAGGCCCAGGCTGGGACCTACACCTGCCATATCCATCTGCAGG
AACAGCAGCTCAATGCCACTGTCACATTGGCAATCATCACAGTGACTCC
CAAATCCTTTGGGTCACCTGGATCCCTGGGGAAGCTGCTTTGTGAGGTG
ACTCCAGTATCTGGACAAGAACGCTTTGTGTGGAGCTCTCTGGACACCC
CATCCCAGAGGAGTTTCTCAGGACCTTGGCTGGAGGCACAGGAGGCCCA
GCTCCTTTCCCAGCCTTGGCAATGCCAGCTGTACCAGGGGGAGAGGCTT
CTTGGAGCAGCAGTGTACTTCACAGAGCTGTCTAGCCCAGGTGCCCAAC
GCTCTGGGAGAGCCCCAGGTGCCCTCCCAGCAGGCCACCTCGTTGCCGC
CATCCTGGGCCTGGGCCTGGTGCTGGGGCTGCTGGGCCCCCTGGCCATC
CTGCTGGCCCTGTACCTGCTCCGGAGGGACCAGAGGCTGCCCCCCGATG
CCCACAAGCCCCCTGGGGGAGGCAGTTTCCGGACCCCCATCCAAGAGGA
GCAGGCCGACGCCCACTCCACCCTGGCCAAGATC.

Therefore, in some embodiments, the chimeric receptor is a P2A-CD8-CD112R-β/IL2-15 fusion and has the amino acid sequence: GSGATNFSLLKQAGDVEENPGPMALPVTALLLPLALLLHAARPTPEVWVQVRMEATEL SSFTIRCGFLGSGSISLVTVSWGGPNGAGGTTLAVLHPERGIRQWAPARQARWETQS SISLILEGSGASSPCANTTFCCKFASFPEGSWEACGSLPPSSDPGLSAPPTPAPILRADL IPWLGHLLVGLSGAFGFIILVYLLINCRNTGPWLKKVLKCNTPDPSKFFSQLSSEHGGDV QKWLSSPFPSSSFSPGGLAPEISPLEVLERDKVTQLLLQQDKVPEPASLSSNHSLTSCF TNQGYFFFHLPDALEIEACQVYFTYDPYSEEDPDEGVAGAPTGSSPQPLQPLSGEDDA YCTFPSRDDLLLFSPSLLGGPSPPSTAPGGSGAGEERMPPSLQERVPRDWDPQPLGP PTPGVPDLVDFQPPPELVLREAGEEVPDAGPREGVSFPWSRPPGQGEFRALNARLPL NTDAYLSLQELQGQDPTHLV (SEQ ID NO:108, CD112R-β/IL2-15), with the following structure: P2A-CD8a SP-CD112R extracellular domain-IL2Rβ TM domain-IL2Rβ intracellular domain.

Therefore, in some embodiments, the chimeric receptor is encoded by the nucleic acid sequence:

(SEQ ID NO: 109)
GGCAGCGGCGCCACCAACTTCAGCCTGCTGAAGCAGGCCGGCGACGTGG
AGGAGAACCCCGGCCCCATGGCCTTACCAGTGACCGCCTTGCTCCTGCC
GCTGGCCTTGCTGCTCCACGCCGCCAGGCCGACCCCGGAGGTGTGGGTT
CAAGTTCGGATGGAGGCCACCGAGCTCTCGTCCTTCACCATCCGTTGTG
GGTTCCTGGGGTCTGGCTCCATCTCCCTGGTGACTGTGAGCTGGGGGGG
CCCCAACGGTGCTGGGGGGACCACGCTGGCTGTGTTGCACCCAGAACGT
GGCATCCGGCAATGGGCCCCTGCTCGCCAGGCCCGCTGGGAAACCCAGA
GCAGCATCTCTCTCATCCTGGAAGGCTCTGGGGCCAGCAGCCCCTGCGC
CAACACCACCTTCTGCTGCAAGTTTGCGTCCTTCCCTGAGGGCTCCTGG
GAGGCCTGTGGGAGCCTCCCGCCCAGCTCAGACCCAGGGCTCTCTGCCC
CGCCGACTCCTGCCCCCATTCTGCGGGCAGACCTGATTCCGTGGCTCGG
CCACCTCCTCGTGGGCCTCAGCGGGGCTTTTGGCTTCATCATCTTAGTG
TACTTGCTGATCAACTGCAGGAACACCGGGCCATGGCTGAAGAAGGTCC
TGAAGTGTAACACCCCAGACCCCTCGAAGTTCTTTTCCCAGCTGAGCTC
AGAGCATGGAGGAGACGTCCAGAAGTGGCTCTCTTCGCCCTTCCCCTCA
TCGTCCTTCAGCCCTGGCGGCCTGGCACCTGAGATCTCGCCACTAGAAG
TGCTGGAGAGGGACAAGGTGACGCAGCTGCTCCTGCAGCAGGACAAGGT
GCCTGAGCCCGCATCCTTAAGCAGCAACCACTCGCTGACCAGCTGCTTC
ACCAACCAGGGTTACTTCTTCTTCCACCTCCCGGATGCCTTGGAGATAG
AGGCCTGCCAGGTGTACTTTACTTACGACCCCTACTCAGAGGAAGACCC
TGATGAGGGTGTGGCCGGGGCACCCACAGGGTCTTCCCCCCAACCCCTG
CAGCCTCTGTCAGGGGAGGACGACGCCTACTGCACCTTCCCCTCCAGGG
ATGACCTGCTGCTCTTCTCCCCCAGTCTCCTCGGTGGCCCCAGCCCCCC
AAGCACTGCCCCTGGGGGCAGTGGGGCCGGTGAAGAGAGGATGCCCCCT
TCTTTGCAAGAAAGAGTCCCCAGAGACTGGGACCCCCAGCCCCTGGGGC
CTCCCACCCCAGGAGTCCCAGACCTGGTGGATTTTCAGCCACCCCCTGA
GCTGGTGCTGCGAGAGGCTGGGGAGGAGGTCCCTGACGCTGGCCCCAGG
GAGGGAGTCAGTTTCCCCTGGTCCAGGCCTCCTGGGCAGGGGGAGTTCA
GGGCCCTTAATGCTCGCCTGCCCCTGAACACTGATGCCTACTTGTCCCT
CCAAGAACTCCAGGGTCAGGACCCAACTCACTTGGTG.

### Chimeric Antigen Receptor

CARs generally incorporate an antigen recognition domain from the single-chain variable fragments (scFv) of a monoclonal antibody (mAb) with transmembrane signaling motifs involved in lymphocyte activation (Sadelain M, et al. Nat Rev Cancer 2003 3:35-45). Disclosed herein are chimeric antigen receptor (CAR) that can be that can be expressed in immune effector cells to suppress alloreactive donor cells.

The disclosed CAR is generally made up of three domains: an ectodomain, a transmembrane domain, and an endodomain. The ectodomain comprises the EGFR or MUC1-binding region and is responsible for antigen recognition. It also optionally contains a signal peptide (SP) so that the CAR can be glycosylated and anchored in the cell membrane of the immune effector cell. The transmembrane domain (TD), is as its name suggests, connects the ectodomain to the endodomain and resides within the cell membrane when expressed by a cell. The endodomain is the business end of the CAR that transmits an activation signal to the immune effector cell after antigen recognition. For example, the endodomain can contain an intracellular signaling domain (ISD) and optionally a co-stimulatory signaling region (CSR).

A “signaling domain (SD)” generally contains immunoreceptor tyrosine-based activation motifs (ITAMs) that activate a signaling cascade when the ITAM is phosphorylated. The term “co-stimulatory signaling region (CSR)” refers to intracellular signaling domains from costimulatory protein receptors, such as CD28, 41BB, and ICOS, that are able to enhance T-cell activation by T-cell receptors.

In some embodiments, the endodomain contains an SD or a CSR, but not both. In these embodiments, an immune effector cell containing the disclosed CAR is only activated if another CAR (or a T-cell receptor) containing the missing domain also binds its respective antigen.

In some embodiments, the disclosed CAR is defined by the formula:

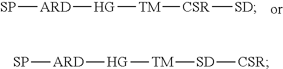

wherein “SP” represents an optional signal peptide,
wherein “ARD” represents an antigen recognition domain,
wherein “HG” represents an optional hinge domain,
wherein “TM” represents a transmembrane domain,
wherein “CSR” represents one or more co-stimulatory signaling regions,
wherein “SD” represents a signaling domain, and
wherein “-” represents a peptide bond or linker.

Additional CAR constructs are described, for example, in Fresnak A D, et al. Engineered T cells: the promise and challenges of cancer immunotherapy. Nat Rev Cancer. 2016 Aug. 23; 16(9):566-81, which is incorporated by reference in its entirety for the teaching of these CAR models.

For example, the CAR can be a TRUCK, Universal CAR, Self-driving CAR, Armored CAR, Self-destruct CAR, Conditional CAR, Marked CAR, TanCAR, Dual CAR, or sCAR.

CAR T cells engineered to be resistant to immunosuppression (Armored CARs) may be genetically modified to no longer express various immune checkpoint molecules (for example, cytotoxic T lymphocyte-associated antigen 4 (CTLA4) or programmed cell death protein 1 (PD1)), with an immune checkpoint switch receptor, or may be administered with a monoclonal antibody that blocks immune checkpoint signaling.

A self-destruct CAR may be designed using RNA delivered by electroporation to encode the CAR. Alternatively, inducible apoptosis of the T cell may be achieved based on ganciclovir binding to thymidine kinase in gene-modified lymphocytes or the more recently described system of activation of human caspase 9 by a small-molecule dimerizer.

A conditional CAR T cell is by default unresponsive, or switched ‘off’, until the addition of a small molecule to complete the circuit, enabling full transduction of both signal 1 and signal 2, thereby activating the CAR T cell. Alternatively, T cells may be engineered to express an adaptor-specific receptor with affinity for subsequently administered secondary antibodies directed at target antigen.

A tandem CAR (TanCAR) T cell expresses a single CAR consisting of two linked single-chain variable fragments (scFvs) that have different affinities fused to intracellular co-stimulatory domain(s) and a CD3ζ domain. TanCAR T cell activation is achieved only when target cells co-express both targets.

A dual CAR T cell expresses two separate CARs with different ligand binding targets; one CAR includes only the CD3ζ domain and the other CAR includes only the co-stimulatory domain(s). Dual CAR T cell activation requires co-expression of both targets.

A safety CAR (sCAR) consists of an extracellular scFv fused to an intracellular inhibitory domain. SCAR T cells co-expressing a standard CAR become activated only when encountering target cells that possess the standard CAR target but lack the sCAR target.

The antigen recognition domain of the disclosed CAR is usually an scFv. There are however many alternatives. An antigen recognition domain from native T-cell receptor (TCR) alpha and beta single chains have been described, as have simple ectodomains (e.g. CD4 ectodomain to recognize HIV infected cells) and more exotic recognition components such as a linked cytokine (which leads to recognition of cells bearing the cytokine receptor). In fact almost anything that binds a given target with high affinity can be used as an antigen recognition region.

The endodomain is the business end of the CAR that after antigen recognition transmits a signal to the immune effector cell, activating at least one of the normal effector functions of the immune effector cell. Effector function of a T cell, for example, may be cytolytic activity or helper activity including the secretion of cytokines. Therefore, the endodomain may comprise the “intracellular signaling domain” of a T cell receptor (TCR) and optional co-receptors. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal.

Cytoplasmic signaling sequences that regulate primary activation of the TCR complex that act in a stimulatory manner may contain signaling motifs which are known as immunoreceptor tyrosine-based activation motifs (ITAMs). Examples of ITAM containing cytoplasmic signaling sequences include those derived from CD8, CD3ζ, CD3δ, CD3γ, CD3ε, CD32 (Fc gamma RIIa), DAP10, DAP12, CD79a, CD79b, FcγRIγ, FcγRIIIγ, FcεRIβ (FCERIB), and FcεRIγ (FCERIG).

In particular embodiments, the intracellular signaling domain is derived from CD3 zeta (CD3ζ) (TCR zeta, GenBank accno. BAG36664.1). T-cell surface glycoprotein CD3 zeta (CD3ζ) chain, also known as T-cell receptor T3 zeta chain or CD247 (Cluster of Differentiation 247), is a protein that in humans is encoded by the CD247 gene.

First-generation CARs typically had the intracellular domain from the CD3ζ chain, which is the primary transmitter of signals from endogenous TCRs. Second-generation CARs add intracellular signaling domains from various costimulatory protein receptors (e.g., CD28, 41BB, ICOS) to the endodomain of the CAR to provide additional signals to the T cell. More recent, third-generation CARs combine multiple signaling domains to further augment potency. T cells grafted with these CARs have demonstrated improved expansion, activation, persistence, and tumor-eradicating efficiency independent of costimulatory receptor/ligand interaction (Imai C, et al. Leukemia 2004 18:676-84; Maher J, et al. Nat Biotechnol 2002 20:70-5).

For example, the endodomain of the CAR can be designed to comprise the CD3ζ signaling domain by itself or combined with any other desired cytoplasmic domain(s) useful in the context of the CAR of the invention. For example, the cytoplasmic domain of the CAR can comprise a CD3ζ chain portion and a costimulatory signaling region. The costimulatory signaling region refers to a portion of the CAR comprising the intracellular domain of a costimulatory molecule. A costimulatory molecule is a cell surface molecule other than an antigen receptor or their ligands that is required for an efficient response of lymphocytes to an antigen. Examples of such molecules include CD27, CD28, 4-1BB (CD137), OX40, CD30, CD40, ICOS, lymphocyte function-associated antigen-1 (LFA-1), CD2, CD7, LIGHT, NKG2C, B7-H3, and a ligand that specifically binds with CD123, CD8, CD4, b2c, CD80, CD86, DAP10, DAP12, MyD88, BTNL3, and NKG2D. Thus, while the CAR is exemplified primarily with CD28 as the co-stimulatory signaling element, other costimulatory elements can be used alone or in combination with other co-stimulatory signaling elements.

In some embodiments, the CAR comprises a hinge sequence. A hinge sequence is a short sequence of amino acids that facilitates antibody flexibility (see, e.g., Woof et al., Nat. Rev. Immunol., 4(2): 89-99 (2004)). The hinge sequence may be positioned between the antigen recognition moiety (e.g., scFv) and the transmembrane domain. The hinge sequence can be any suitable sequence derived or obtained from any suitable molecule. In some embodiments, for example, the hinge sequence is derived from a CD8a molecule or a CD28 molecule.

The transmembrane domain may be derived either from a natural or from a synthetic source. Where the source is natural, the domain may be derived from any membrane-bound or transmembrane protein. For example, the transmembrane region may be derived from (i.e. comprise at least the transmembrane region(s) of) the alpha, beta or zeta chain of the T-cell receptor, CD28, CD3 epsilon, CD45, CD4, CD5, CD8 (e.g., CD8 alpha, CD8 beta), CD9, CD16, CD22, CD33, CD37, CD64, CD80, CD86, CD134, CD137, or CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, and PAG/Cbp. Alternatively the transmembrane domain may be synthetic, in which case it will comprise predominantly hydrophobic residues such as leucine and valine. In some cases, a triplet of phenylalanine, tryptophan and valine will be found at each end of a synthetic transmembrane domain. A short oligo- or polypeptide linker, such as between 2 and 10 amino acids in length, may form the linkage between the transmembrane domain and the endoplasmic domain of the CAR.

In some embodiments, the CAR has more than one transmembrane domain, which can be a repeat of the same transmembrane domain, or can be different transmembrane domains.

In some embodiments, the CAR is a multi-chain CAR, as described in WO2015/039523, which is incorporated by reference for this teaching. A multi-chain CAR can comprise separate extracellular ligand binding and signaling domains in different transmembrane polypeptides. The signaling domains can be designed to assemble in juxtamembrane position, which forms flexible architecture closer to natural receptors, that confers optimal signal transduction. For example, the multi-chain CAR can comprise a part of an FCERI alpha chain and a part of an FCERI beta chain such that the FCERI chains spontaneously dimerize together to form a CAR.

In some embodiments, the antigen recognition domain is single chain variable fragment (scFv) antibody. The affinity/specificity of an scFv is driven in large part by specific sequences within complementarity determining regions (CDRs) in the heavy (V_{H}) and light (V_{L}) chain. Each V_{H} and V_{L} sequence will have three CDRs (CDR1, CDR2, CDR3).

In some embodiments, the antigen recognition domain is derived from natural antibodies, such as monoclonal antibodies. In some cases, the antibody is human. In some cases, the antibody has undergone an alteration to render it less immunogenic when administered to humans. For example, the alteration comprises one or more techniques selected from the group consisting of chimerization, humanization, CDR-grafting, deimmunization, and mutation of framework amino acids to correspond to the closest human germline sequence.

In some embodiments, the tumor antigen is MUC1 (TA-MUC1). Anti-MUC1 antibodies are disclosed in U.S. Patent Publication 2017/0204191A1, which is incorporated by reference for these antibodies, including sequences for use in preparing scFVs.

In some embodiments of the anti-MUC1 scFv, the CDR1 sequence of the V_{H} domain comprises the amino acid sequence NYGMN (SEQ ID NO:74), GYAMS (SEQ ID NO:75), or R/GYA/GMS (SEQ ID NO:76); CDR2 sequence of the V_{H} domain comprises the amino acid sequence WINTYTGEPTYA/VG/DDFKG (SEQ ID NO:77) or TISSGGTYIYYPDSVKG (SEQ ID NO:78); CDR3 sequence of the V_{H} domain comprises the amino acid sequence S/TGT/DT/AXXY/FYA (SEQ ID NO:79), TGTTAILNG (SEQ ID NO:80), SGDGYWYYA (SEQ ID NO:81) or DNYGXXYDYG/A (SEQ ID NO:82); CDR1 sequence of the V_{L} comprises the amino acid sequence SASSSV/ISYM/IH/Y (SEQ ID NO:83) or RASKSVSTSGYSYMH (SEQ ID NO:84); CDR2 sequence of the V_{L} domain comprises the amino acid sequence S/GTSNLAS (SEQ ID NO:85) or LASNLES (SEQ ID NO:86); and CDR3 sequence of the V_{L} domain comprises the amino acid sequence QQRSS/NYPS/FT (SEQ ID NO:87) or QHSRELPFT (SEQ ID NO:88).

In some embodiments, the anti-MUC1 scFv V_{H} domain comprises the amino acid

(SEQ ID NO: 89)
VQLQESGGGLVQPGGSMKLSCVASGFTFSNYWMNWVRQSPEKGLEWVAE
IRLKSNNYATHYAESVKGRFTISRDDSKSSVYLQMNNLRAEDTGIYYCT
GVGQFAYWGQGTTVTVSS.

In some embodiments, the anti-MUC1 scFv V_{H} domain comprises the amino acid sequence:

(SEQ ID NO: 90)
DIELTQESALTTSPGETVTLTCRSSTGAVTTSNYANWVQEKPDHLFTGL
IGGTNNRAPGVPARFSGSLIGDKAALTITGAQTEDEAIYFCALWYSNHW
VFGGGTKL.

In some embodiments, the anti-MUC1 scFv V_{L} domain comprises the amino acid sequence:

(SEQ ID NO: 91)
DIELTQESALTTSPGETVTLTCRSSTGAVTTSNYANWVQEKPDHLFTGL
IGGTNNRAPGVPARFSGSLIGDKAALTITGAQTEDEAIYFCALWYSNHW
VFGGGTKL.

In some embodiments, the anti-MUC1 scFv V_{L} domain comprises the amino acid sequence:

(SEQ ID NO: 92)
GGGGSVQLQESGGGLVQPGGSMKLSCVASGFTFSNYWMNWVRQSPEKGL
EWVAEIRLKSNNYATHYAESVKGRFTISRDDSKSSVYLQMNNLRAEDTG
IYYCTGVGQFAYWGQGTTVTVSS.

In some embodiments, the anti-MUC1 comprises an amino acid sequence:

(SEQ ID NO: 93)
VQLQESGGGLVQPGGSMKLSCVASGFTFSNYWMNWVRQSPEKGLEWVAE
IRLKSNNYATHYAESVKGRFTISRDDSKSSVYLQMNNLRAEDTGIYYCT
GVGQFAYWGQGTTVTVSSGGGGSGGGGSGGGGSDIELTQESALTTSPGE
TVTLTCRSSTGAVTTSNYANWVQEKPDHLFTGLIGGTNNRAPGVPARFS
GSLIGDKAALTITGAQTEDEAIYFCALWYSNHWVFGGGTKL.

In some embodiments, the anti-MUC1 comprises an amino acid sequence:

(SEQ ID NO: 94)
EIVLTQSPATLSLSPGERATLTCSATSSVSYIHWYQQRPGQSPRLLIYS
TSNLASGIPARFSGSGSGSDYTLTISSLEPEDFAVYYCQQRSSSPFTFG
SGTKVEIKGGGGSGGGGSGGGGSEVQLVESGGGLVKPGGSLRLSCAASG
FTFSRYGMSWVRQAPGKRLEWVSTISGGGTYIYYPDSVKGRFTISRDNA
KNTLYLQMNSLRAEDTAVYYCTRDNYGRNYDYGMDYWGQGTLVTVSS.

In some embodiments the anti-MUC1 scFv is derived from a SM3 anti-MUC1 antibody. Therefore, in some embodiments of the anti-MUC1 scFv, the CDR1 sequence of the V_{H} domain comprises the amino acid sequence GFTFSNYWMN (SEQ ID NO:110); CDR2 sequence of the V_{H} domain comprises the amino acid sequence RLKSNNYATHYAES (SEQ ID NO:111); CDR3 sequence of the V_{H} domain comprises the amino acid sequence VGQFAY (SEQ ID NO: 112); CDR1 sequence of the V_{L} comprises the amino acid sequence STGAVTTSNYAN (SEQ ID NO: 113); CDR2 sequence of the V_{L} domain comprises the amino acid sequence GTNNRAP (SEQ ID NO:114); and CDR3 sequence of the V_{L} domain comprises the amino acid sequence ALWYSNHWV (SEQ ID NO:115).

In some embodiments, the anti-MUC1 scFv V_{H} domain comprises the amino acid sequence:

(SEQ ID NO: 116)
QVQLQESGGGLVQPGGSMKLSCVASGFTFSNYWMNWVRQSPEKGLEWVA
EIRLKSNNYATHYAESVKGRFTISRDDSKSSVYLQMNNLRAEDTGIYYC
TGVGQFAYWGQGTTVTVSSAKTTPPTVYPLAPGSNAASQSMVTLGCLVK
GYFPEPVTVTWNSGSLASGVHTFPAVLQSDLYTLSSSVTVPSSTWPSET
VTCNVAHPASSTKVDAKIVPRD.

In some embodiments, the anti-MUC1 scFv V_{L} domain comprises the amino acid sequence:

(SEQ ID NO: 117)
DIVVTQESALTTSPGETVTLTCRSSTGAVTTSNYANWVQEKPDHLFTGL
IGGTNNRAPGVPARFSGSLIGDKAALTITGAQTEDEAIYFCALWYSNHW
VFGGGTKLTVLGSEKSSPSVTLFPPSSEELETNKATLVCTITDFYPGVV
TVDWKVDGTPVTQGMETTQPSKQSNNKYMASSYLTLTARAWERHSSYSC
QVTHEGHTVEKSLSRADCS.

In some embodiments of the anti-MUC1 scFv, the CDR1 sequence of the V_{H} domain comprises the amino acid sequence YTFTDHAIH (SEQ ID NO: 118); CDR2 sequence of the V_{H} domain comprises the amino acid sequence WIGHFSPGNTDIKY (SEQ ID NO: 119); CDR3 sequence of the V_{H} domain comprises the amino acid sequence KTSTFFFD (SEQ ID NO: 120); CDR1 sequence of the V_{L} comprises the amino acid sequence SQSLLNSGDQKNY (SEQ ID NO: 121); CDR2 sequence of the V_{L} domain comprises the amino acid sequence KLLIFWASTR (SEQ ID NO: 122); and CDR3 sequence of the V_{L} domain comprises the amino acid sequence CONDYSY (SEQ ID NO:123).

In some embodiments, the anti-MUC1 scFv V_{H} domain comprises the amino acid sequence:

(SEQ ID NO: 124)
VQLQQSDAELVKPGSSVKISCKASGYTFTDHAIHWVKQKPEQGLEWIGH
FSPGNTDIKYNDKFKGKATLTVDRSSSTAYMQLNSLTSEDSAVYFCKTS
TFFFDYWGQGTTLTVSS.

In some embodiments, the anti-MUC1 scFv V_{L} domain comprises the amino acid

(SEQ ID NO: 125)
ELVMTQSPSSLTVTAGEKVTMICKSSQSLLNSGDQKNYLTWYQQKPGQP
PKLLIFWASTRESGVPDRFTGSGSGTDFTLTISSVQAEDLAVYYCQNDY
SYPLTFGAGTKLELK.

In some embodiments, the anti-MUC1 comprises an amino acid sequence:

(SEQ ID NO: 126)
QVQLQQSDAELVKPGSSVKISCKASGYTFTDHAIHWVKQKPEQGLEWIG
HFSPGNTDIKYNDKFKGKATLTVDRSSSTAYMQLNSLTSEDSAVYFCKT
STFFFDYWGQGTTLTVSSGGGGSGGGGSGGGGSELVMTQSPSSLTVTAG
EKVTMICKSSQSLLNSGDQKNYLTWYQQKPGQPPKLLIFWASTRESGVP
DRFTGSGSGTDFTLTISSVQAEDLAVYYCQNDYSYPLTFGAGTKLELK.

In some embodiments, the CAR polypeptide has the amino acid sequence: MLRLLLALNLFPSIQVTG-QVQLQQSDAELVKPGSSVKISCKASGYTFTDHAIHWVKQKPEQGLEWIGHFSPGNTDI KYNDKFKGKATLTVDRSSSTAYMQLNSLTSEDSAVYFCKTSTFFFDYWGQGTTLTVSS GGGGSGGGGSGGGGSELVMTQSPSSLTVTAGEKVTMICKSSQSLLNSGDQKNYLTW YQQKPGQPPKLLIFWASTRESGVPDRFTGSGSGTDFTLTISSVQAEDLAVYYCONDYS YPLTFGAGTKLELK-IEVMYPPPYLDNEKSNGTIIHVKGKHLCPSPLFPGPSKPFWVLVVVGGVLACYSLLVTVA FIIFWV-RSKRSRLLHSDYMNMTPRRPGPTRKHYQPYAPPRDFAAYRS-KRGRKKLLYIFKQPFMRPVQTTQEEDGCSCRFPEEEEGGCEL-RVKFSRSADAPAYQQGQNQLYNELNLGRREEYDVLDKRRGRDPEMGGKPQRRKNPQ EGLYNELQKDKMAEAYSEIGMKGERRRGKGHDGLYQGLSTATKDTYDALHMQALPPR (SEQ ID NO: 127, Muc1-BB28z), with the following structure CD28 SP-5E5 scFv-CD28 linker-CD28 TM-CD28 CSD-4-1-BB-CD3z.

In some embodiments, the CAR polypeptide has the amino acid sequence: MLRLLLALNLFPSIQVTG-QVQLQQSDAELVKPGSSVKISCKASGYTFTDHAIHWVKQKPEQGLEWIGHFSPGNTDI KYNDKFKGKATLTVDRSSSTAYMQLNSLTSEDSAVYFCKTSTFFFDYWGQGTTLTVSS GGGGSGGGGSGGGGSELVMTQSPSSLTVTAGEKVTMICKSSQSLLNSGDQKNYLTW YQQKPGQPPKLLIFWASTRESGVPDRFTGSGSGTDFTLTISSVQAEDLAVYYCONDYS YPLTFGAGTKLELK-IEVMYPPPYLDNEKSNGTIIHVKGKHLCPSPLFPGPSKPFWVLVVVGGVLACYSLLVTVA FIIFWV-RSKRSRLLHSDYMNMTPRRPGPTRKHYQPYAPPRDFAAYRS-KRGRKKLLYIFKQPFMRPVQTTQEEDGCSCRFPEEEEGGCEL-RVKFSRSADAPAYQQGQNQLYNELNLGRREEYDVLDKRRGRDPEMGGKPQRRKNPQ EGLYNELQKDKMAEAYSEIGMKGERRRGKGHDGLYQGLSTATKDTYDALHMQALPPR-GSGSG-MVSKGEELFTGVVPILVELDGDVNGHKFSVRGEGEGDATNGKLTLKFICTSGKLPVPW PTLVTTLSYGVQCFARYPDHMKQHDFFKSAMPEGYVQERTISFKDDGSYRTRAEVKFE GDTLVNRIELKGIDFKEDGNILGHKLEYNMNVWDAYITADKQKNGIKANFKIEHNVEDGG VQLADAYQQNTPIGDGSVLLPDNHYLSFQSKLFKDPNEQRDHMVLLEFVTAAGITPGM DELYK (SEQ ID NO:128, Muc1-BB28z-ametrine), with the following structure CD28 SP-5E5 scFv-CD28 linker-CD28 TM-CD28 CSD-4-1-BB-CD3z-Ametrine.

In some embodiments, the CAR polypeptide is co-expressed with the switch polypeptide as the amino acid sequence: MLRLLLALNLFPSIQVTGQVQLQQSDAELVKPGSSVKISCKASGYTFTDHAIHWVKQKP EQGLEWIGHFSPGNTDIKYNDKFKGKATLTVDRSSSTAYMQLNSLTSEDSAVYFCKTST FFFDYWGQGTTLTVSSGGGGSGGGGSGGGGSELVMTQSPSSLTVTAGEKVTMICKS SQSLLNSGDQKNYLTWYQQKPGQPPKLLIFWASTRESGVPDRFTGSGSGTDFTLTISS VQAEDLAVYYCONDYSYPLTFGAGTKLELKIEVMYPPPYLDNEKSNGTIIHVKGKHLCP SPLFPGPSKPFWVLVVVGGVLACYSLLVTVAFIIFWVRSKRSRLLHSDYMNMTPRRPG PTRKHYQPYAPPRDFAAYRSKRGRKKLLYIFKQPFMRPVQTTQEEDGCSCRFPEEEE GGCELRVKFSRSADAPAYQQGQNQLYNELNLGRREEYDVLDKRRGRDPEMGGKPQR RKNPQEGLYNELQKDKMAEAYSEIGMKGERRRGKGHDGLYQGLSTATKDTYDALHM QALPPR-GSGATNFSLLKQAGDVEENPGPMALPVTALLLPLALLLHAARPTPEVWVQVRMEATEL SSFTIRCGFLGSGSISLVTVSWGGPNGAGGTTLAVLHPERGIRQWAPARQARWETQS SISLILEGSGASSPCANTTFCCKFASFPEGSWEACGSLPPSSDPGLSAPPTPAPILRADL IPWLGHLLVGLSGAFGFIILVYLLINCRNTGPWLKKVLKCNTPDPSKFFSQLSSEHGGDV QKWLSSPFPSSSFSPGGLAPEISPLEVLERDKVTQLLLQQDKVPEPASLSSNHSLTSCF TNQGYFFFHLPDALEIEACQVYFTYDPYSEEDPDEGVAGAPTGSSPQPLQPLSGEDDA YCTFPSRDDLLLFSPSLLGGPSPPSTAPGGSGAGEERMPPSLQERVPRDWDPQPLGP PTPGVPDLVDFQPPPELVLREAGEEVPDAGPREGVSFPWSRPPGQGEFRALNARLPL NTDAYLSLQELQGQDPTHLV (SEQ ID NO: 129, Muc1-BB28z+CD112R^{ED}-IL2Rβ^{CSD}), with the following structure CD28 SP-5E5 scFv-CD28 linker-CD28 TM-CD28 CSD-4-1-BB-CD3z.

shRNA Knock-Down

In some embodiments, the disclosed compositions and methods involve knocking down one or more of the inhibitory receptors, including LAG-3, CD112R, TIGIT, TIM3 and DR5 using shRNA.

In some embodiments, the shRNA inhibits LAG-3 (Accession No: X51985) which in some embodiments has the nucleic acid sequence:

(SEQ ID NO: 95)
ATGTGGGAGGCTCAGTTCCTGGGCTTGCTGTTTCTGCAGCCGCTTTGGG
TGGCTCCAGTGAAGCCTCTCCAGCCAGGGGCTGAGGTCCCGGTGGTGTG
GGCCCAGGAGGGGGCTCCTGCCCAGCTCCCCTGCAGCCCCACAATCCCC
CTCCAGGATCTCAGCCTTCTGCGAAGAGCAGGGGTCACTTGGCAGCATC
AGCCAGACAGTGGCCCGCCCGCTGCCGCCCCCGGCCATCCCCTGGCCCC
CGGCCCTCACCCGGCGGCGCCCTCCTCCTGGGGGCCCAGGCCCCGCCGC
TACACGGTGCTGAGCGTGGGTCCCGGAGGCCTGCGCAGCGGGAGGCTGC
CCCTGCAGCCCCGCGTCCAGCTGGATGAGCGCGGCCGGCAGCGCGGGGA
CTTCTCGCTATGGCTGCGCCCAGCCCGGCGCGCGGACGCCGGCGAGTAC
CGCGCCGCGGTGCACCTCAGGGACCGCGCCCTCTCCTGCCGCCTCCGTC
TGCGCCTGGGCCAGGCCTCGATGACTGCCAGCCCCCCAGGATCTCTCAG
AGCCTCCGACTGGGTCATTTTGAACTGCTCCTTCAGCCGCCCTGACCGC
CCAGCCTCTGTGCATTGGTTCCGGAACCGGGGCCAGGGCCGAGTCCCTG
TCCGGGAGTCCCCCCATCACCACTTAGCGGAAAGCTTCCTCTTCCTGCC
CCAAGTCAGCCCCATGGACTCTGGGCCCTGGGGCTGCATCCTCACCTAC
AGAGATGGCTTCAACGTCTCCATCATGTATAACCTCACTGTTCTGGGTC
TGGAGCCCCCAACTCCCTTGACAGTGTACGCTGGAGCAGGTTCCAGGGT
GGGGCTGCCCTGCCGCCTGCCTGCTGGTGTGGGGACCCGGTCTTTCCTC
ACTGCCAAGTGGACTCCTCCTGGGGGAGGCCCTGACCTCCTGGTGACTG
GAGACAATGGCGACTTTACCCTTCGACTAGAGGATGTGAGCCAGGCCCA
GGCTGGGACCTACACCTGCCATATCCATCTGCAGGAACAGCAGCTCAAT
GCCACTGTCACATTGGCAATCATCACAGTGACTCCCAAATCCTTTGGGT
CACCTGGATCCCTGGGGAAGCTGCTTTGTGAGGTGACTCCAGTATCTGG
ACAAGAACGCTTTGTGTGGAGCTCTCTGGACACCCCATCCCAGAGGAGT
TTCTCAGGACCTTGGCTGGAGGCACAGGAGGCCCAGCTCCTTTCCCAGC
CTTGGCAATGCCAGCTGTACCAGGGGGAGAGGCTTCTTGGAGCAGCAGT
GTACTTCACAGAGCTGTCTAGCCCAGGTGCCCAACGCTCTGGGAGAGCC
CCAGGTGCCCTCCCAGCAGGCCACCTCCTGCTGTTTCTCACCCTTGGTG
TCCTTTCTCTGCTCCTTTTGGTGACTGGAGCCTTTGGCTTTCACCTTTG
GAGAAGACAGTGGCGACCAAGACGATTTTCTGCCTTAGAGCAAGGGATT
CACCCTCCGCAGGCTCAGAGCAAGATAGAGGAGCTGGAGCAAGAACCGG
AGCCGGAGCCGGAGCCGGAACCGGAGCCCGAGCCCGAGCCCGAGCCGGA
GCAGCTCTGA.

In some embodiments, the shRNA inhibits CD112R (Accession No: BC073861) which in some embodiments has the nucleic acid sequence:

(SEQ ID NO: 96)
ATGAGAACAGAGGCACAGGTGCCGGCCCTGCAGCCCCCAGAACCTGGAC
TGGAGGGGGCCATGGGGCACCGGACCCTGGTCCTGCCCTGGGTGCTGCT
GACCTTGTGTGTCACTGCGGGGACCCCGGAGGTGTGGGTTCAAGTTCGG
ATGGAGGCCACCGAGCTCTCGTCCTTCACCATCCGTTGTGGGTTCCTGG
GGTCTGGCTCCATCTCCCTGGTGACTGTGAGCTGGGGGGGCCCCAACGG
TGCTGGGGGGACCACGCTGGCTGTGTTGCACCCAGAACGTGGCATCCGG
CAATGGGCCCCTGCTCGCCAGGCCCGCTGGGAAACCCAGAGCAGCATCT
CTCTCATCCTGGAAGGCTCTGGGGCCAGCAGCCCCTGCGCCAACACCAC
CTTCTGCTGCAAGTTTGCGTCCTTCCCTGAGGGCTCCTGGGAGGCCTGT
GGGAGCCTCCCGCCCAGCTCAGACCCAGGGCTCTCTGCCCCGCCGACTC
CTGCCCCCATTCTGCGGGCAGACCTGGCCGGGATCTTGGGGGTCTCAGG
AGTCCTCCTCTTTGGCTGTGTCTACCTCCTTCATCTGCTGCGCCGACAT
AAGCACCGCCCTGCCCCTAGGCTCCAGCCGTCCCGCACCAGCCCCCAGG
CACCGAGAGCACGAGCATGGGCACCAAGCCAGGCCTCCCAGGCTGCTCT
TCACGTCCCTTATGCCACTATCAACACCAGCTGCCGCCCAGCTACTTTG
GACACAGCTCACCCCCATGGGGGGCCGTCCTGGTGGGCGTCACTCCCCA
CCCACGCTGCACACCGGCCCCAGGGCCCTGCCGCCTGGGCCTCCACACC
CATCCCTGCACGTGGCAGCTTTGTCTCTGTTGAGAATGGACTCTACGCT
CAGGCAGGGGAGAGGCCTCCTCACACTGGTCCCGGCCTCACTCTTTTCC
CTGACCCTCGGGGGCCCAGGGCCATGGAAGGACCCTTAGGAGTTCGATG
A.

In some embodiments, the shRNA inhibits TIGIT (Accession No: AK097192.1) which in some embodiments has the nucleic acid sequence:

(SEQ ID NO: 97)
ATGCGCTGGTGTCTCCTCCTGATCTGGGCCCAGGGGCTGAGGCAGGCTC
CCCTCGCCTCAGGAATGATGACAGGCACAATAGAAACAACGGGGAACAT
TTCTGCAGAGAAAGGTGGCTCTATCATCTTACAATGTCACCTCTCCTCC
ACCACGGCACAAGTGACCCAGGTCAACTGGGAGCAGCAGGACCAGCTTC
TGGCCATTTGTAATGCTGACTTGGGGTGGCACATCTCCCCATCCTTCAA
GGATCGAGTGGCCCCAGGTCCCGGCCTGGGCCTTACCCTCCAGTCGCTG
ACCGTGAACGATACAGGGGAGTACTTCTGCATCTATCACACCTACCCTG
ATGGGGCGTACACTGGGAGAATCTTCCTGGAGGTCCTAGAAAGCTCAGT
GGCTGAGCACGGTGCCAGGTTCCAGATTCCATTGCTTGGAGCCATGGCC
GCGACGCTGGTGGTCATCTGCACAGCAGTCATCGTGGTGGTCGCGTTGA
CTAGAAAGAAGAAAGCCCTCAGAATCCATTCTGTGGAAGGTGACCTCAG
GAGAAAATCAGCTGGACAGGAGGAATGGAGCCCCAGTGCTCCCTCACCC
CCAGGAAGCTGTGTCCAGGCAGAAGCTGCACCTGCTGGGCTCTGTGGAG
AGCAGCGGGGAGAGGACTGTGCCGAGCTGCATGACTACTTCAATGTCCT
GAGTTACAGAAGCCTGGGTAACTGCAGCTTCTTCACAGAGACTGGTTA
G.

In some embodiments, the shRNA inhibits TIM3 (Accession No: AF251707) which in some embodiments has the nucleic acid sequence:

(SEQ ID NO: 98)
ATGTTTTCACATCTTCCCTTTGACTGTGTCCTGCTGCTGCTGCTGCTAC
TACTTACAAGGTCCTCAGAAGTGGAATACAGAGCGGAGGTCGGTCAGAA
TGCCTATCTGCCCTGCTTCTACACCCCAGCCGCCCCAGGGAACCTCGTG
CCCGTCTGCTGGGGCAAAGGAGCCTGTCCTGTGTTTGAATGTGGCAACG
TGGTGCTCAGGACTGATGAAAGGGATGTGAATTATTGGACATCCAGATA
CTGGCTAAATGGGGATTTCCGCAAAGGAGATGTGTCCCTGACCATAGAG
AATGTGATTCTAGCAGACAGTGGGATCTACTGCTGCCGGATCCAAATCC
CAGGCATAATGAATGATGAAAAATTTAACCTGAAGTTGGTCATCAAACC
AGCCAAGGTCACCCCTGCACCGACTCTGCAGAGAGACTTCACTGCAGCC
TTTCCAAGGATGCTTACCACCAGGGGACATGGCCCAGCAGAGACACAGA
CACTGGGGAGCCTCCCTGATATAAATCTAACACAAATATCCACATTGGC
CAATGAGTTACGGGACTCTAGATTGGCCAATGACTTACGGGACTCTGGA
GCAACCATCAGAATAGGCATCTACATCGGAGCAGGGATCTGTGCTGGGC
TGGCTCTGGCTCTTATCTTCGGCGCTTTAATTTTCAAATGGTATTCTCA
TAGCAAAGAGAAGATACAGAATTTAAGCCTCATCTCTTTGGCCAACCTC
CCTCCCTCAGGATTGGCAAATGCAGTAGCAGAGGGAATTCGCTCAGAAG
AAAACATCTATACCATTGAAGAGAACGTATATGAAGTGGAGGAGCCCAA
TGAGTATTATTGCTATGTCAGCAGCAGGCAGCAACCCTCACAACCTTTG
GGTTGTCGCTTTGCAATGCCATAG.

In some embodiments, the shRNA inhibits DR5 (Accession No: AF016849) which in some embodiments has the nucleic acid sequence:

(SEQ ID NO: 99)
ATGGAACAACGGGGACAGAACGCCCCGGCCGCTTCGGGGGCCCGGAAAA
GGCACGGCCCAGGACCCAGGGAGGCGCGGGGAGCCAGGCCTGGGCCCCG
GGTCCCCAAGACCCTTGTGCTCGTTGTCGCCGCGGTCCTGCTGTTGGTC
TCAGCTGAGTCTGCTCTGATCACCCAACAAGACCTAGCTCCCCAGCAGA
GAGCGGCCCCACAACAAAAGAGGTCCAGCCCCTCAGAGGGATTGTGTCC
ACCTGGACACCATATCTCAGAAGACGGTAGAGATTGCATCTCCTGCAAA
TATGGACAGGACTATAGCACTCACTGGAATGACCTCCTTTTCTGCTTGC
GCTGCACCAGGTGTGATTCAGGTGAAGTGGAGCTAAGTCCGTGCACCAC
GACCAGAAACACAGTGTGTCAGTGCGAAGAAGGCACCTTCCGGGAAGAA
GATTCTCCTGAGATGTGCCGGAAGTGCCGCACAGGGTGTCCCAGAGGGA
TGGTCAAGGTCGGTGATTGTACACCCTGGAGTGACATCGAATGTGTCCA
CAAAGAATCAGGTACAAAGCACAGTGGGGAAGCCCCAGCTGTGGAGGAG
ACGGTGACCTCCAGCCCAGGGACTCCTGCCTCTCCCTGTTCTCTCTCAG
GCATCATCATAGGAGTCACAGTTGCAGCCGTAGTCTTGATTGTGGCTGT
GTTTGTTTGCAAGTCTTTACTGTGGAAGAAAGTCCTTCCTTACCTGAAA
GGCATCTGCTCAGGTGGTGGTGGGGACCCTGAGCGTGTGGACAGAAGCT
CACAACGACCTGGGGCTGAGGACAATGTCCTCAATGAGATCGTGAGTAT
CTTGCAGCCCACCCAGGTCCCTGAGCAGGAAATGGAAGTCCAGGAGCCA
GCAGAGCCAACAGGTGTCAACATGTTGTCCCCCGGGGAGTCAGAGCATC
TGCTGGAACCGGCAGAAGCTGAAAGGTCTCAGAGGAGGAGGCTGCTGGT
TCCAGCAAATGAAGGTGATCCCACTGAGACTCTGAGACAGTGCTTCGAT
GACTTTGCAGACTTGGTGCCCTTTGACTCCTGGGAGCCGCTCATGAGGA
AGTTGGGCCTCATGGACAATGAGATAAAGGTGGCTAAAGCTGAGGCAGC
GGGCCACAGGGACACCTTGTACACGATGCTGATAAAGTGGGTCAACAAA
ACCGGGCGAGATGCCTCTGTCCACACCCTGCTGGATGCCTTGGAGACGC
TGGGAGAGAGACTTGCCAAGCAGAAGATTGAGGACCACTTGTTGAGCTC
TGGAAAGTTCATGTATCTAGAAGGTAATGCAGACTCTGCCATGTCCTA
A.

### Nucleic Acids and Vectors

Also disclosed are polynucleotides and polynucleotide vectors encoding the disclosed chimeric receptors, CAR polypeptides, and/or shRNA that allow expression of the chimeric receptors, CAR polypeptides, and/or shRNA in the disclosed immune effector cells.

Nucleic acid sequences encoding the disclosed chimeric receptors, CAR polypeptides, and/or shRNA, and regions thereof, can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

Expression of nucleic acids encoding chimeric receptors, CAR polypeptides, and/or shRNA is typically achieved by operably linking a nucleic acid encoding the chimeric receptors, CAR polypeptides, and/or shRNA to a promoter, and incorporating the construct into an expression vector. Typical cloning vectors contain transcription and translation terminators, initiation sequences, and promoters useful for regulation of the expression of the desired nucleic acid sequence.

The disclosed nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. In some embodiments, the polynucleotide vectors are lentiviral or retroviral vectors.

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo.

In some embodiments, the disclosed chimeric receptor, CAR polypeptide, and/or shRNA are expressed in the immune effector cell using a retroviral vector. For example, in some embodiments, the vector is a pMXs (Cell Biolabs, Inc.) retroviral vector based on Moloney murine leukemia virus (MMLV). In some embodiments, the vector is a pDON-AI, pMEI-5 or pDON-5 (Takara Bio Inc.) retroviral vector contain only LTR and packaging signal (Ψ sequence) of MoMLV genome, but not gag, pol, and env coding sequence.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, MND (myeloproliferative sarcoma virus) promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. The promoter can alternatively be an inducible promoter. Examples of inducible promoters include, but are not limited to a metallothionine promoter, a glucocorticoid promoter, a progesterone promoter, and a tetracycline promoter.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another.

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the different fluorescent protein gene. Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York).

Biological methods for introducing a polynucleotide of interest into a host cell include the use of DNA and RNA vectors. Viral vectors, and especially retroviral vectors, have become the most widely used method for inserting genes into mammalian, e.g., human cells.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes. Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc, (Birmingham, Ala.).

### Immune Effector Cells

Also disclosed are immune effector cells that are engineered to express the disclosed chimeric receptors, CAR polypeptides, and/or shRNA (also referred to herein as “CAR-T cells”). These cells are preferably obtained from the subject to be treated (i.e. are autologous). However, in some embodiments, immune effector cell lines or donor effector cells (allogeneic) are used. Immune effector cells can be obtained from a number of sources, including peripheral blood mononuclear cells, bone marrow, lymph node tissue, cord blood, thymus tissue, tissue from a site of infection, ascites, pleural effusion, spleen tissue, and tumors. Immune effector cells can be obtained from blood collected from a subject using any number of techniques known to the skilled artisan, such as Ficoll™ separation. For example, cells from the circulating blood of an individual may be obtained by apheresis. In some embodiments, immune effector cells are isolated from peripheral blood lymphocytes by lysing the red blood cells and depleting the monocytes, for example, by centrifugation through a PERCOLL™ gradient or by counterflow centrifugal elutriation. A specific subpopulation of immune effector cells can be further isolated by positive or negative selection techniques. For example, immune effector cells can be isolated using a combination of antibodies directed to surface markers unique to the positively selected cells, e.g., by incubation with antibody-conjugated beads for a time period sufficient for positive selection of the desired immune effector cells. Alternatively, enrichment of immune effector cells population can be accomplished by negative selection using a combination of antibodies directed to surface markers unique to the negatively selected cells.

In some embodiments, the immune effector cells comprise any leukocyte involved in defending the body against infectious disease and foreign materials. For example, the immune effector cells can comprise lymphocytes, monocytes, macrophages, dendritic cells, mast cells, neutrophils, basophils, eosinophils, or any combinations thereof. For example, the immune effector cells can comprise T lymphocytes.

T cells or T lymphocytes can be distinguished from other lymphocytes, such as B cells and natural killer cells (NK cells), by the presence of a T-cell receptor (TCR) on the cell surface. They are called T cells because they mature in the thymus (although some also mature in the tonsils). There are several subsets of T cells, each with a distinct function.

T helper cells (T_{H} cells) assist other white blood cells in immunologic processes, including maturation of B cells into plasma cells and memory B cells, and activation of cytotoxic T cells and macrophages. These cells are also known as CD4+ T cells because they express the CD4 glycoprotein on their surface. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including T_{H}1, T_{H}2, T_{H}3, T_{H}17, T_{H}9, or T_{FH}, which secrete different cytokines to facilitate a different type of immune response.

Cytotoxic T cells (T_{C} cells, or CTLs) destroy virally infected cells and tumor cells, and are also implicated in transplant rejection. These cells are also known as CD8⁺ T cells since they express the CD8 glycoprotein at their surface. These cells recognize their targets by binding to antigen associated with MHC class I molecules, which are present on the surface of all nucleated cells. Through IL-10, adenosine and other molecules secreted by regulatory T cells, the CD8+ cells can be inactivated to an anergic state, which prevents autoimmune diseases.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory cells may be either CD4⁺ or CD8⁺. Memory T cells typically express the cell surface protein CD45RO.

Regulatory T cells (T_{reg} cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus. Two major classes of CD4⁺ T_{reg} cells have been described—naturally occurring T_{reg} cells and adaptive T_{reg} cells.

Natural killer T (NKT) cells (not to be confused with natural killer (NK) cells) bridge the adaptive immune system with the innate immune system. Unlike conventional T cells that recognize peptide antigens presented by major histocompatibility complex (MHC) molecules, NKT cells recognize glycolipid antigen presented by a molecule called CD1d.

In some embodiments, the T cells comprise a mixture of CD4+ cells. In other embodiments, the T cells are enriched for one or more subsets based on cell surface expression. For example, in some cases, the T comprise are cytotoxic CD8⁺ T lymphocytes. In some embodiments, the T cells comprise γδ T cells, which possess a distinct T-cell receptor (TCR) having one γ chain and one δ chain instead of α and β chains.

Natural-killer (NK) cells are CD56⁺CD3⁻ large granular lymphocytes that can kill virally infected and transformed cells, and constitute a critical cellular subset of the innate immune system (Godfrey J, et al. Leuk Lymphoma 2012 53:1666-1676). Unlike cytotoxic CD8+T lymphocytes, NK cells launch cytotoxicity against tumor cells without the requirement for prior sensitization, and can also eradicate MHC-I-negative cells (Narni-Mancinelli E, et al. Int Immunol 2011 23:427-431). NK cells are safer effector cells, as they may avoid the potentially lethal complications of cytokine storms (Morgan R A, et al. Mol Ther 2010 18:843-851), tumor lysis syndrome (Porter D L, et al. N Engl J Med 2011 365:725-733), and on-target, off-tumor effects.

### Therapeutic Methods

Immune effector cells expressing the disclosed chimeric receptors, CAR polypeptides, and/or shRNA can be used to treat cancers, such as TNBCs. The cancer of the disclosed methods can be any tumor antigen-expressing cell in a subject undergoing unregulated growth, invasion, or metastasis. In some aspects, the cancer can be any neoplasm or tumor for which radiotherapy is currently used. Alternatively, the cancer can be a neoplasm or tumor that is not sufficiently sensitive to radiotherapy using standard methods. Thus, the cancer can be a sarcoma, lymphoma, leukemia, carcinoma, blastoma, or germ cell tumor. A representative but non-limiting list of cancers that the disclosed compositions can be used to treat include lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, endometrial cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer.

In some aspects, the cancer is an epithelial cancer, such as breast, gastric, colorectal, cholangiocarcinoma, ovarian, or non-small cell lung cancer.

The disclosed CAR-T cells may be administered either alone, or as a pharmaceutical composition in combination with diluents and/or with other components such as IL-2, IL-15, or other cytokines or cell populations.

In some embodiments, the disclosed CAR-T cells are administered in combination with a vascular endothelial growth factor-A (VEGF-A) or vascular endothelial growth factor receptor (VEGFR) antagonist antibody. VEGF inhibitors utilized in oncologic care include bevacizumab, sorafenib, sunitinib, nilotinib, pazopanib, and dasatinib.

Briefly, pharmaceutical compositions may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions for use in the disclosed methods are in some embodiments formulated for intravenous administration. Pharmaceutical compositions may be administered in any manner appropriate treat MM. The quantity and frequency of administration will be determined by such factors as the condition of the patient, and the severity of the patient's disease, although appropriate dosages may be determined by clinical trials.

When a “therapeutic amount” is indicated, the precise amount of the compositions of the present invention to be administered can be determined by a physician with consideration of individual differences in age, weight, extent of transplantation, and condition of the patient (subject). It can generally be stated that a pharmaceutical composition comprising the T cells described herein may be administered at a dosage of 10⁴ to 10⁹ cells/kg body weight, such as 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319: 1676, 1988). The optimal dosage and treatment regime for a particular patient can readily be determined by one skilled in the art of medicine by monitoring the patient for signs of disease and adjusting the treatment accordingly.

The administration of the disclosed compositions may be carried out in any convenient manner, including by injection, transfusion, or implantation. The compositions described herein may be administered to a patient subcutaneously, intradermally, intranodally, intramedullary, intramuscularly, by intravenous (i.v.) injection, or intraperitoneally. In some embodiments, the disclosed compositions are administered to a patient by intradermal or subcutaneous injection. In some embodiments, the disclosed compositions are administered by i.v. injection. The compositions may also be injected directly into a site of transplantation.

In certain embodiments, the disclosed CAR-T cells are administered to a patient in conjunction with (e.g., before, simultaneously or following) any number of relevant treatment modalities, including but not limited to thalidomide, dexamethasone, bortezomib, and lenalidomide. In further embodiments, the CAR-modified immune effector cells may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. In some embodiments, the CAR-modified immune effector cells are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions of the present invention are administered following B-cell ablative therapy such as agents that react with CD20, e.g., Rituxan. For example, in some embodiments, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

One primary concern with CAR-T cells as a form of “living therapeutic” is their manipulability in vivo and their potential immune-stimulating side effects. To better control CAR-T therapy and prevent against unwanted side effects, a variety of features have been engineered including off-switches, safety mechanisms, and conditional control mechanisms. Both self-destruct and marked/tagged CAR-T cells for example, are engineered to have an “off-switch” that promotes clearance of the CAR-expressing T-cell. A self-destruct CAR-T contains a CAR, but is also engineered to express a pro-apoptotic suicide gene or “elimination gene” inducible upon administration of an exogenous molecule. A variety of suicide genes may be employed for this purpose, including HSV-TK (herpes simplex virus thymidine kinase), Fas, iCasp9 (inducible caspase 9), CD20, MYC TAG, and truncated EGFR (endothelial growth factor receptor). HSK for example, will convert the prodrug ganciclovir (GCV) into GCV-triphosphate that incorporates itself into replicating DNA, ultimately leading to cell death. iCasp9 is a chimeric protein containing components of FK506-binding protein that binds the small molecule AP1903, leading to caspase 9 dimerization and apoptosis. A marked/tagged CAR-T cell however, is one that possesses a CAR but also is engineered to express a selection marker. Administration of a mAb against this selection marker will promote clearance of the CAR-T cell. Truncated EGFR is one such targetable antigen by the anti-EGFR mAb, and administration of cetuximab works to promotes elimination of the CAR-T cell. CARs created to have these features are also referred to as sCARs for ‘switchable CARs’, and RCARs for ‘regulatable CARs’. A “safety CAR”, also known as an “inhibitory CAR” (iCAR), is engineered to express two antigen binding domains. One of these extracellular domains is directed against a first antigen and bound to an intracellular costimulatory and stimulatory domain. The second extracellular antigen binding domain however is specific for normal tissue and bound to an intracellular checkpoint domain such as CTLA4, PD1, or CD45. Incorporation of multiple intracellular inhibitory domains to the iCAR is also possible. Some inhibitory molecules that may provide these inhibitory domains include B7-H1, B7-1, CD160, PIH, 2B4, CEACAM (CEACAM-1. CEACAM-3, and/or CEACAM-5), LAG-3, TIGIT, BTLA, LAIR1, and TGFβ-R. In the presence of normal tissue, stimulation of this second antigen binding domain will work to inhibit the CAR. It should be noted that due to this dual antigen specificity, iCARs are also a form of bi-specific CAR-T cells. The safety CAR-T engineering enhances specificity of the CAR-T cell for tissue, and is advantageous in situations where certain normal tissues may express very low levels of an antigen that would lead to off target effects with a standard CAR (Morgan 2010). A conditional CAR-T cell expresses an extracellular antigen binding domain connected to an intracellular costimulatory domain and a separate, intracellular costimulator. The costimulatory and stimulatory domain sequences are engineered in such a way that upon administration of an exogenous molecule the resultant proteins will come together intracellularly to complete the CAR circuit. In this way, CAR-T activation can be modulated, and possibly even ‘fine-tuned’ or personalized to a specific patient. Similar to a dual CAR design, the stimulatory and costimulatory domains are physically separated when inactive in the conditional CAR; for this reason these too are also referred to as a “split CAR”.

Typically, CAR-T cells are created using α-β T cells, however γ-δ T cells may also be used. In some embodiments, the described CAR constructs, domains, and engineered features used to generate CAR-T cells could similarly be employed in the generation of other types of CAR-expressing immune cells including NK (natural killer) cells, B cells, mast cells, myeloid-derived phagocytes, and NKT cells. Alternatively, a CAR-expressing cell may be created to have properties of both T-cell and NK cells. In an additional embodiment, the transduced with CARs may be autologous or allogeneic.

Several different methods for CAR expression may be used including retroviral transduction (including γ-retroviral), lentiviral transduction, transposon/transposases (Sleeping Beauty and PiggyBac systems), and messenger RNA transfer-mediated gene expression. Gene editing (gene insertion or gene deletion/disruption) has become of increasing importance with respect to the possibility for engineering CAR-T cells as well. CRISPR-Cas9, ZFN (zinc finger nuclease), and TALEN (transcription activator like effector nuclease) systems are three potential methods through which CAR-T cells may be generated.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Development of CAR-T Cell for Triple Negative Breast Cancer (TNBC) Patients

Tumor-host immune interactions are considered prominent drivers of cancer progression. Recent evidence of the interplay between the immune system and the disease course of TNBC has led to the identification of TNBC as an immunogenic malignancy (Dees, S., et al., Mol Cancer Ther, 2020. 19(12):2409-2421; Tafreshi, N. K., et al. World J Clin Oncol, 2020. 11(4): 169-179). The recent FDA approval of atezolizumab in combination with the chemotherapeutic agent nabpaclitaxel for the treatment of PD-L1-positive unresectable, locally advanced, or metastatic TNBC has fueled an immunotherapy era in TNBC (Schmid, P., et al., Lancet Oncol, 2020. 21(1):44-59). A peripheral blood T cell modified to express a CAR is referred to as a CAR-T cell, reviewed by (Dees, S., et al., Mol Cancer Ther, 2020. 19(12): 2409-2421; Bagley, S. J., et al., Pharmacol Ther, 2020. 205:107419; June, C. H., et al., Science, 2018. 359(6382): 1361-1365; Rossig, C., Clin Immunol, 2018. 186:54-58). A typical ectodomain of a CAR usually comprises of a tumor antigen specific antibody-derived recognition motif such as a single-chain variable fragment (scFv). The scFv drives specificity of the CAR (Dees, S., et al., Mol Cancer Ther, 2020. 19(12):2409-2421; June, C. H., et al., Science, 2018. 359(6382): 1361-1365). The most prominent successes of CAR-T cell therapy have been documented in hematologic cancers with the FDA approvals of Kymriah and Yescarta, two second-generation CAR-T cell products targeting the B-cell antigen CD19 (Han, D., et al., J Cancer, 2021. 12(2):326-334; Yang, X., et al. Curr Med Sci, 2019. 39(6):874-882). The quantity and quality of TNBC immunotherapy research being conducted in the CAR-T cell space has exponentially increased over the last 3 years (Dees, S., et al., Mol Cancer Ther, 2020. 19(12): 2409-2421; Li, Z., et al., J Transl Med, 2018. 16(1): 147). The antitumor activity associated with targeting over a dozen TNBC antigens with CAR-T cells has been demonstrated in preclinical in vitro and preclinical in vivo studies. Identification of cell-surface markers with high and broad expression among TR-TNBC will allow for the development of novel CAR-T cell therapies that provide promising immunotherapeutic strategy to improve the survival rates of patients with local as well as metastatic TNBC.

Although TNBC targets have been published in the literature, a systematic approach toward identification and validation of therapy resistant TNBC (TR-TNBC) drug targets has not been reported. Discovery of targets that are specific for CR-TNBC is a first and necessary step toward development of effective CAR-T for this currently untreatable disease. By using TCGA RNA seq data from the TNBC positive tumors and comparing to normal tissues (brain, heart, liver, lung, muscle, nerve, pancreases, spleen, bladder, blood vessel, colon, GI, kidneys) and also using literature and protein atlas data base, we have identified several markers that have high expression in TNBC samples. These cell surface genes were selected based on level and breadth of differential expression in TNBC, ability to make scFv, and known biological function. The cell surface genes that are potential targets for TNBC include LAMP2, CDH3, GABRP, FOLR1, VTCN1, SLC39A6, CDCP1, EPHB3, TACSTD2 and MUC1. The RNA seq data was also examined for immune check point ligands, chemokines, interleukins, pH and hypoxia related genes and T-cells inhibitory products related genes (e.g. Arginase-1, CD37 and CD39, . . . ) and a set of the genes was chosen for target discovery (based on the high expression in TNBC patients but low expression in normal tissues) and a set of genes for microenvironment (Table 1). Notably, LAMP2 expression is high in normal tissues too. The reason that this was chosen in the panel for TNBC TMA staining is because of translocation of this protein from lysosome (in normal cells) to cell membrane in breast cancer cells (Damaghi, M., et al., Nat Commun, 2015. 6:8752). This makes this protein a good target for CAR development. Also, the expression of MUC-1 is high in some normal tissues. MUC-1 has been considered an excellent antigen for CAR, because first it is elevated up to 100-fold on cancer tissues. Second, there is aberrant glycosylation of MUC1, such that the O-glycans present on MUC1-expressing cancer cells are shorter compared with those on normal cells, hence, exposing regions of the protein core. In addition, new carbohydrate antigens (e.g., Tn, STn and TF) are exposed (Tang, C. K., et al., Expert Rev Vaccines, 2008. 7(7):951-62). Furthermore, it has been shown that cancer patients generate specific MUC1 T-cell and antibody responses (Domenech, N., et al., J Immunol, 1995. 155(10): 4766-74; Jerome, K. R., et al., J Immunol, 1993. 151(3): 1654-62).

TABLE 1
RNA seq data from TCGA. Different groups of TNBC patients: BL1 and BL2 (basal-like),
luminal androgen receptor (LAR), mesenchymal (M).

| gene_name | BL1_LQ_Tumor | | | BL2_LQ_Tumor | | LAR_LQ_Tumor | | M_LQ_Tumor | | NIH | |
|---|---|---|---|---|---|---|---|---|---|---|---|
| LAMP2 | 6.5 | | | 6.5 | | 6.3 | | 5.9 | | 6.3 | |
| CDH3 (Cadherin 3) | 5.6 | | | 6.1 | | 4.0 | | 5.1 | | 7.7 | |
| GABRP | 7.6 | | | 4.9 | | 1.0 | | 5.2 | | 8.2 | |
| FOLR1 | 2.1 | | | 2.5 | | 0.5 | | 2.9 | | 7.1 | |
| VTCN1 (B7-H4) | 5.3 | | | 4.7 | | 2.8 | | 5.8 | | 3.0 | |
| SLC39A6 (LIV-1) | 5.0 | | | 4.9 | | 4.8 | | 5.0 | | 4.7 | |
| CDCP1 | 3.5 | | | 3.8 | | 2.9 | | 2.6 | | 3.1 | |
| EPHB3 (EPH Receptor | 5.1 | | | 4.6 | | 2.9 | | 6.0 | | 6.1 | |
| B3) | | | | | | | | | | | |
| TACSTD2 (TROP-2) | 6.4 | | | 7.0 | | 6.9 | | 6.6 | | 4.3 | |
| MUC1 | 5.4 | | | 5.9 | | 7.2 | | 4.6 | | 6.5 | |
| TGFB1 | 3.8 | | | 4.3 | | 4.0 | | 3.3 | | 4.2 | |
| VEGFA | 5.5 | | | 5.4 | | 5.1 | | 5.3 | | 9.2 | |
| CD274 (PD-L1) | 1.4 | | | 1.5 | | 1.0 | | 0.4 | | 2.0 | |
| PVRL2 (CD112) | 6.1 | | | 6.1 | | 6.3 | | 6.0 | | 4.6 | |
| LGALS9 (Galectin 9), | 6.0 | | | 6.2 | | 5.9 | | 4.6 | | 5.8 | |
| Tim3 ligand | | | | | | | | | | | |
| IDO1 | 3.5 | | | 4.0 | | 2.5 | | 1.4 | | 1.2 | |
| LGALS3 (Galectin 3), | 6.7 | | | 7.1 | | 7.0 | | 6.3 | | 5.2 | |
| LAG3 ligand | | | | | | | | | | | |
| HMGB1 (Tim3 ligand) | 8.3 | | | 8.0 | | 7.8 | | 8.1 | | 6.9 | |
| TNFSF10 (DR5 ligand) | 5.2 | | | 6.1 | | 5.7 | | 4.2 | | 4.1 | |
| TNFSF10 (DR5 ligand) | 5.2 | | | 6.1 | | 5.7 | | 4.2 | | 4.1 | |
| | | | | | | | | | | | Blood |
| gene_name | Brain | Heart | Liver | Lung | Muscle | Nerve | Pancreas | Spleen | Bladder | Blood | Vessel |
| LAMP2 | 5.8 | 4.7 | 6.0 | 6.2 | 5.5 | 6.1 | 4.7 | 5.5 | 5.8 | 6.1 | 6.1 |
| CDH3 (Cadherin 3) | 2.0 | 0.2 | 0.1 | 2.6 | 0.2 | 2.3 | 0.8 | 4.3 | 3.9 | 0.1 | 0.1 |
| GABRP | 0.1 | 0.4 | 0.8 | 0.6 | 0.2 | 0.7 | 0.3 | 0.1 | 0.4 | 0.0 | 0.0 |
| FOLR1 | 1.0 | 0.4 | 0.2 | 7.3 | 0.1 | 1.5 | 1.7 | 0.2 | 0.4 | 0.1 | 0.1 |
| VTCN1 (B7-H4) | 0.1 | 0.2 | 0.4 | 0.2 | 0.2 | 0.1 | 3.2 | 0.0 | 2.0 | 0.0 | 0.0 |
| SLC39A6 (LIV-1) | 4.4 | 3.0 | 2.3 | 4.0 | 3.0 | 4.6 | 3.2 | 3.4 | 6.2 | 3.1 | 3.1 |
| CDCP1 | 0.4 | 0.3 | 0.3 | 2.7 | 0.3 | 0.8 | 2.2 | 0.8 | 3.3 | 0.6 | 0.6 |
| EPHB3 (EPH Receptor | 2.4 | 1.2 | 0.8 | 2.8 | 0.6 | 3.7 | 3.0 | 1.2 | 2.9 | 0.2 | 0.2 |
| B3) | | | | | | | | | | | |
| TACSTD2 (TROP-2) | 0.4 | 0.4 | 1.6 | 6.4 | 0.5 | 3.6 | 5.3 | 1.1 | 8.5 | 0.8 | 0.8 |
| MUC1 | 2.2 | 2.3 | 1.6 | 9.5 | 1.3 | 6.3 | 7.3 | 3.1 | 6.4 | 1.6 | 1.6 |
| TGFB1 | 3.2 | 4.3 | 3.3 | 6.7 | 2.7 | 5.2 | 3.1 | 6.8 | 5.9 | 7.0 | 7.0 |
| VEGFA | 5.1 | 7.2 | 7.6 | 7.7 | 7.7 | 6.6 | 6.1 | 7.5 | 6.8 | 6.1 | 6.1 |
| CD274 (PD-L1) | 1.2 | 2.5 | 1.1 | 4.8 | 1.2 | 1.7 | 1.2 | 3.7 | 1.3 | 3.8 | 3.8 |
| PVRL2 (CD112) | 3.8 | 5.0 | 6.0 | 6.6 | 3.6 | 6.2 | 4.7 | 6.2 | 5.3 | 3.2 | 3.2 |
| LGALS9 (Galectin 9), | 3.7 | 4.1 | 3.6 | 7.0 | 2.8 | 5.4 | 4.2 | 8.0 | 5.7 | 7.6 | 7.6 |
| Tim3 ligand | | | | | | | | | | | |
| IDO1 | 0.0 | 0.5 | 0.4 | 4.9 | 1.6 | 1.1 | 0.3 | 3.3 | 0.9 | 0.6 | 0.6 |
| LGALS3 (Galectin 3), | 5.4 | 7.0 | 3.3 | 8.4 | 5.4 | 8.7 | 5.5 | 6.0 | 8.7 | 8.6 | 6.3 |
| LAG3 ligand | | | | | | | | | | | |
| HMGB1 (Tim3 ligand) | 7.8 | 6.8 | 6.7 | 8.1 | 7.4 | 8.4 | 6.3 | 8.0 | 8.4 | 8.1 | 7.5 |
| TNFSF10 (DR5 ligand) | 2.7 | 3.5 | 5.2 | 7.1 | 3.1 | 4.9 | 3.6 | 5.8 | 7.1 | 5.8 | 5.9 |
| TNFSF10 (DR5 ligand) | 2.7 | 3.5 | 5.2 | 7.1 | 3.1 | 4.9 | 3.6 | 5.8 | 7.1 | 5.8 | 5.9 |
| gene_name | Breast | Colon | Esophagus | Kidney | Ovary | Pituitary | Skin | Stomach | Thyroid | Uterus | Vagina |
| LAMP2 | 5.9 | 5.3 | 6.0 | 5.9 | 5.7 | 5.4 | 6.6 | 5.3 | 6.1 | 5.8 | 6.3 |
| CDH3 (Cadherin 3) | 4.9 | 0.5 | 4.6 | 3.5 | 6.3 | 3.6 | 4.7 | 1.4 | 2.1 | 5.2 | 4.9 |
| GABRP | 6.3 | 0.3 | 4.2 | 1.4 | 0.2 | 2.0 | 4.1 | 1.0 | 0.2 | 0.7 | 5.7 |
| FOLR1 | 4.2 | 0.3 | 0.4 | 6.1 | 0.4 | 3.8 | 1.7 | 3.9 | 5.7 | 0.4 | 0.3 |
| VTCN1 (B7-H4) | 5.4 | 0.0 | 0.1 | 3.3 | 0.3 | 0.2 | 1.8 | 0.1 | 0.1 | 0.4 | 0.2 |
| SLC39A6 (LIV-1) | 5.7 | 4.0 | 4.1 | 4.0 | 5.3 | 5.0 | 5.6 | 3.6 | 4.3 | 4.7 | 4.5 |
| CDCP1 | 2.4 | 3.6 | 4.7 | 2.2 | 0.5 | 1.5 | 4.2 | 2.9 | 3.3 | 2.7 | 4.5 |
| EPHB3 (EPH Receptor | 4.4 | 3.6 | 4.8 | 1.7 | 3.9 | 2.3 | 5.9 | 4.5 | 1.6 | 4.3 | 5.2 |
| B3) | | | | | | | | | | | |
| TACSTD2 (TROP-2) | 7.4 | 1.0 | 9.4 | 6.5 | 1.3 | 1.8 | 8.6 | 3.6 | 2.9 | 2.7 | 9.2 |
| MUC1 | 7.0 | 7.5 | 7.2 | 9.0 | 4.2 | 4.7 | 4.4 | 10.0 | 6.8 | 6.0 | 6.2 |
| TGFB1 | 5.1 | 5.2 | 5.4 | 5.2 | 4.7 | 4.8 | 5.7 | 4.7 | 5.0 | 6.0 | 6.1 |
| VEGFA | 7.5 | 6.1 | 6.5 | 7.2 | 6.0 | 6.5 | 6.9 | 6.5 | 9.6 | 8.5 | 7.5 |
| CD274 (PD-L1) | 1.7 | 1.3 | 1.7 | 1.6 | 1.4 | 2.5 | 1.3 | 1.3 | 2.3 | 2.0 | 1.6 |
| PVRL2 (CD112) | 6.4 | 5.4 | 5.8 | 6.0 | 5.7 | 5.8 | 5.8 | 5.8 | 6.4 | 5.9 | 5.9 |
| LGALS9 (Galectin 9), | 5.6 | 8.0 | 5.0 | 4.6 | 4.7 | 4.2 | 4.1 | 6.9 | 4.8 | 4.6 | 4.7 |
| Tim3 ligand | | | | | | | | | | | |
| IDO1 | 1.4 | 1.5 | 1.0 | 0.8 | 0.7 | 0.5 | 0.7 | 1.0 | 2.9 | 1.2 | 0.9 |
| LGALS3 (Galectin 3), | 6.3 | 8.7 | 9.5 | 8.2 | 6.6 | 8.2 | 6.7 | 7.6 | 9.1 | 7.5 | 6.8 |
| LAG3 ligand | | | | | | | | | | | |
| HMGB1 (Tim3 ligand) | 7.5 | 8.2 | 8.1 | 8.0 | 7.4 | 8.2 | 7.7 | 8.0 | 7.9 | 7.6 | 7.7 |
| TNFSF10 (DR5 ligand) | 5.9 | 6.4 | 5.2 | 5.6 | 6.3 | 5.0 | 4.1 | 6.3 | 4.9 | 4.4 | 2.2 |
| TNFSF10 (DR5 ligand) | 5.9 | 6.4 | 5.2 | 5.6 | 6.3 | 5.0 | 4.1 | 6.3 | 4.9 | 4.4 | 2.2 |

A TNBC TMA (tissue microarray) was then used to stain the above targets/microenvironmental genes (Table 1) to valid the expression on protein level (ongoing experiment). The TNBC contains 59 treatment-refractory and metaplastic TNBC (n=2-3 punches for each sample), 2 normal breast (n=3 punches for each sample) and normal tissues including spleen, Muscle, kidneys, lung, liver, GI and heart and TNBC cell lines (Hs578t, MDA-mb-231, BT-549. MDA-mb-157).

Since the expression of Galectin-3 (LAG-3 ligand), Galectin-9 (Tim-3 ligand), HMGB1 (Tim-3 ligand), CD112 (CD112R ligand) and TNFSF10 (DR5 ligand) are high in the TCGA RNA seq data (Table 1), the decision was made to work on these receptors for the CAR design. The receptors for these ligands are inhibitory. Therefore, the high expression of these ligands mean that the microenvironment of triple negative breast cancer cells are inhibitory for T-cells. This means that if CAR T cells are not changed, they will not be able to work properly in this inhibitory tumor microenvironment. Therefore, two strategies were developed to manipulate the CAR-T cells.

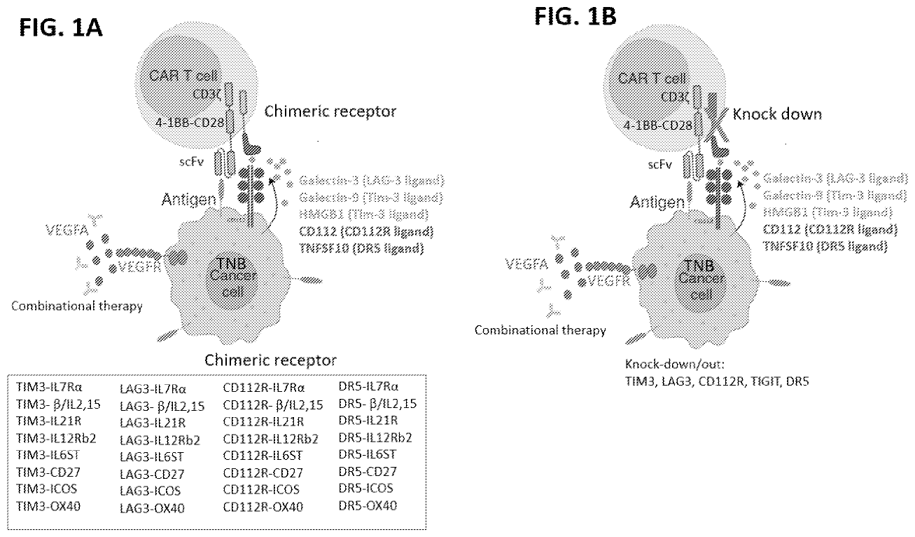

The first strategy involved fusing the extracellular part of the inhibitory receptors (Tim-3, LAG3, CD112R and DR5) to transmembrane and intracellular part of the pro-inflammatory interleukins (IL7Rα, βcommon part of IL2 and IL15, IL21R, IL12Rb2, IL6ST) or co-stimulatory domains (CD27, ICOS, OX40). The other co-stimulatory domains (CD28 and 4-1BB) were added to the CAR itself (FIG. 1A).

The second strategy involves knocking down one or more than one of the inhibitory receptors (FIG. 1B), including LAG-3, CD112R, TIGIT, TIM3 and DR5 using shRNA in the same vector.

The third strategy would be combinational therapy using VEGFA or VEGFR antagonist antibodies, since the concentration of VEGFA is very high in TNBC microenvironment based on the TCGA RNA seq data (Table 1).

### Example 2. Validation on Protein Level

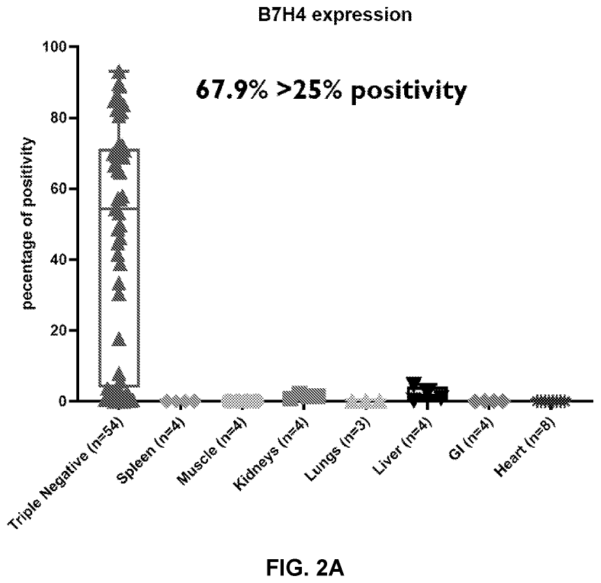

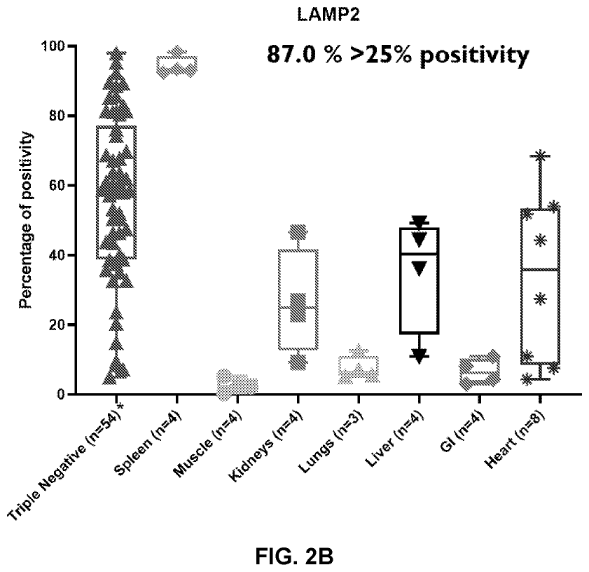

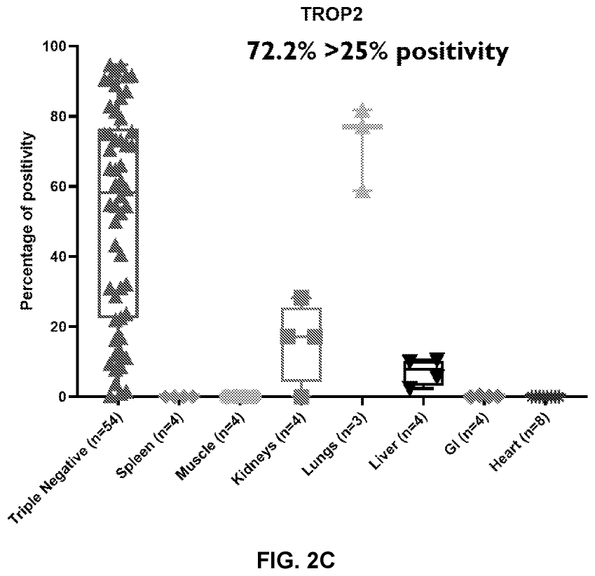

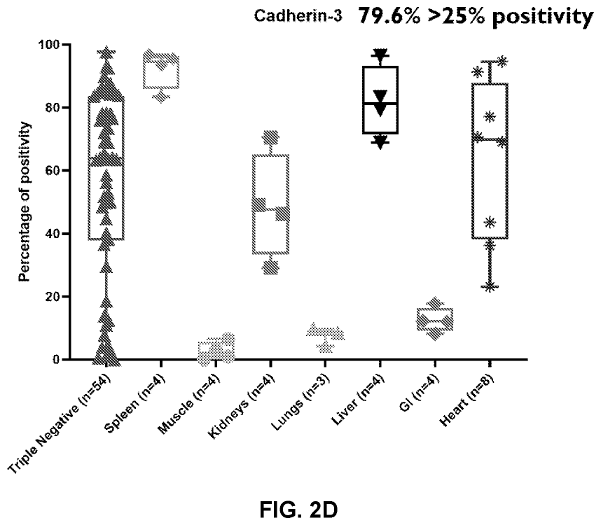

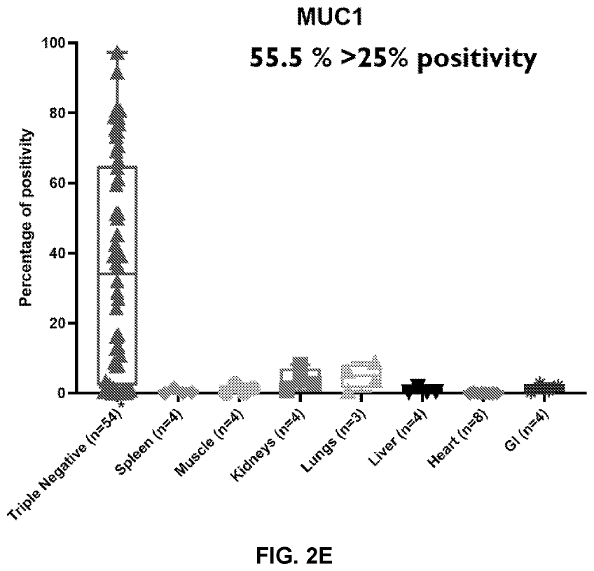

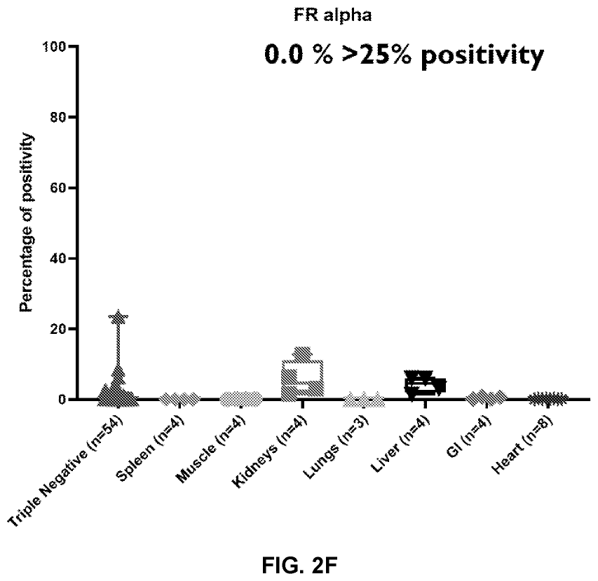

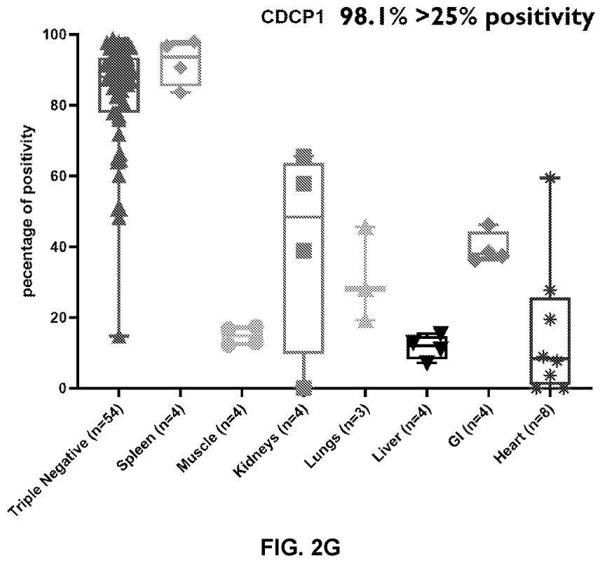

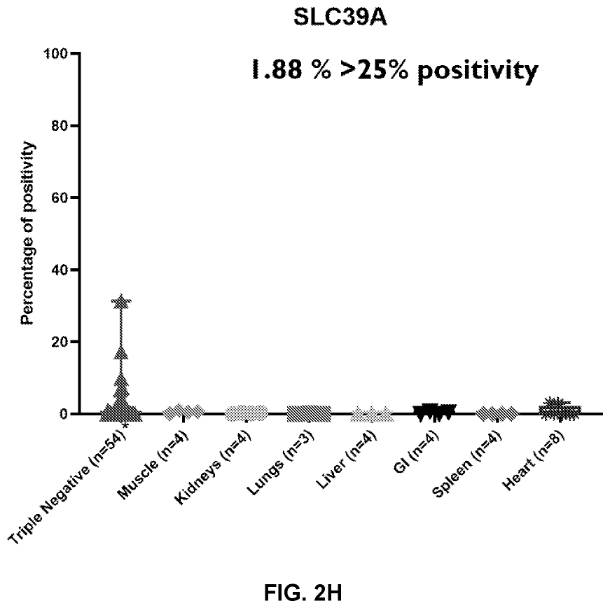

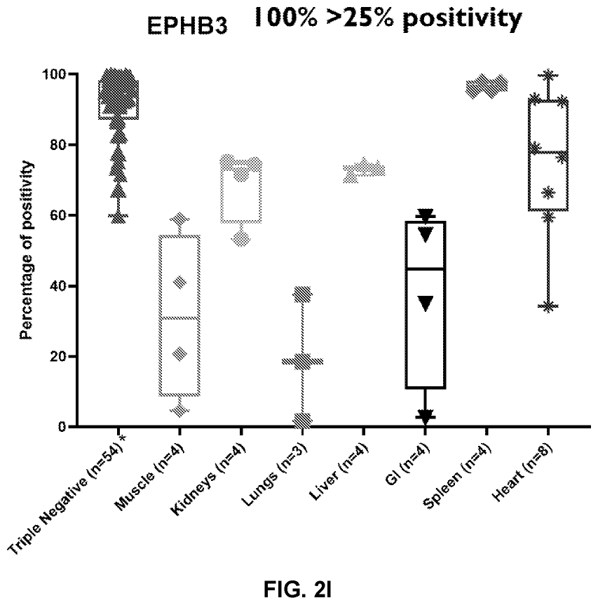

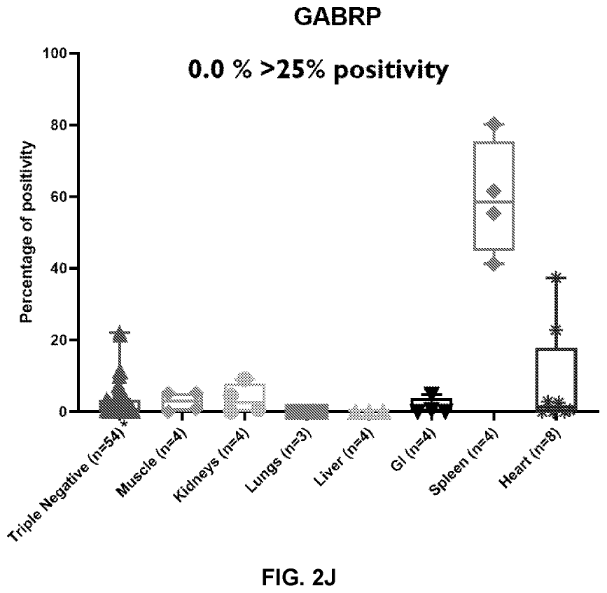

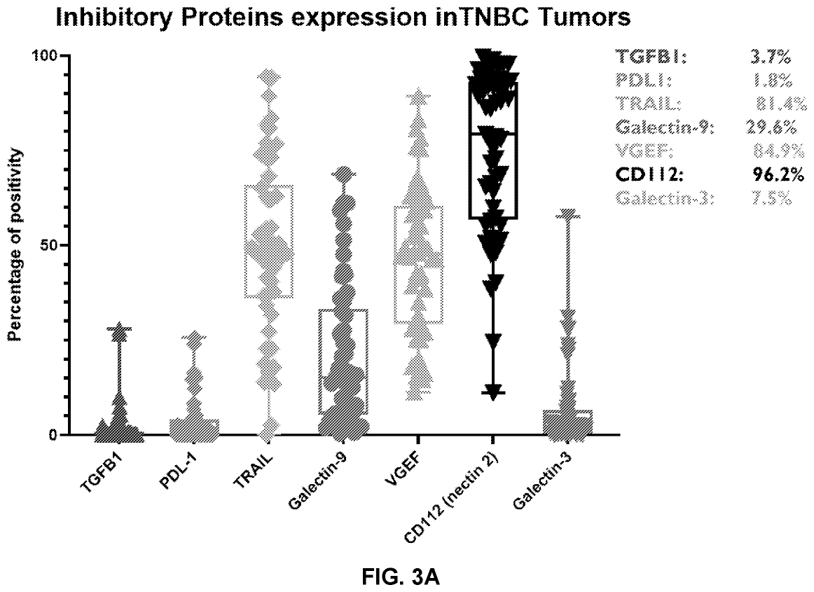

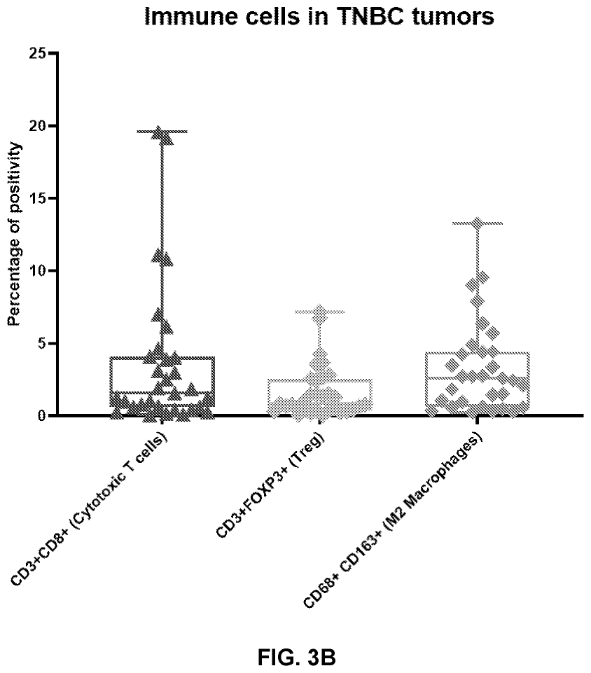

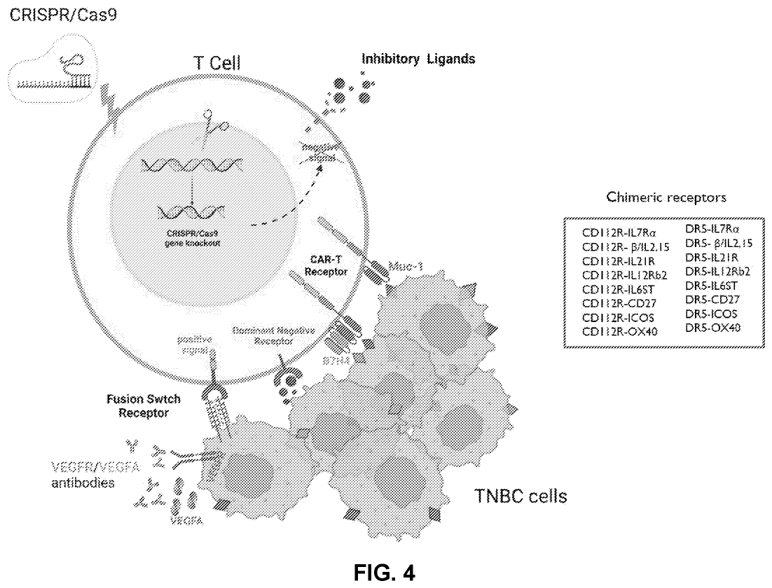

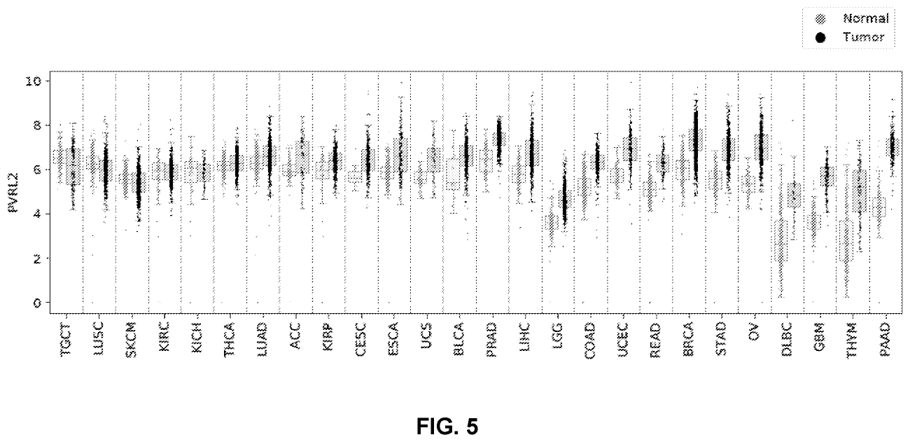

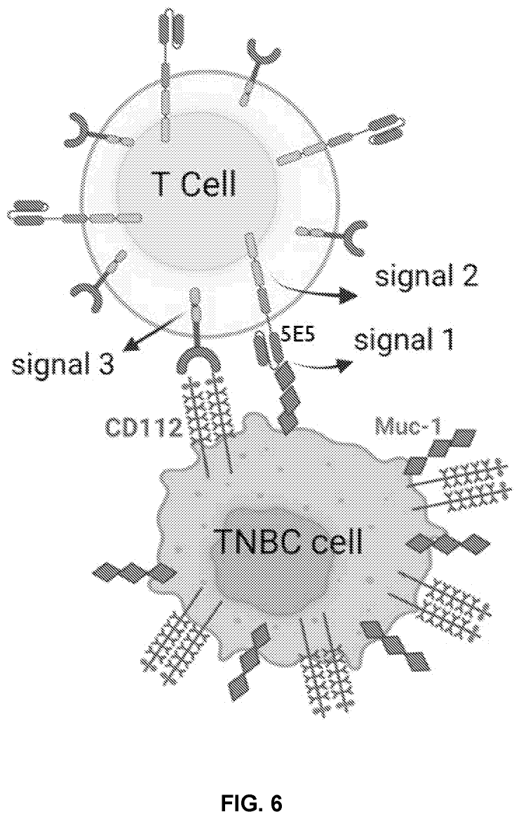

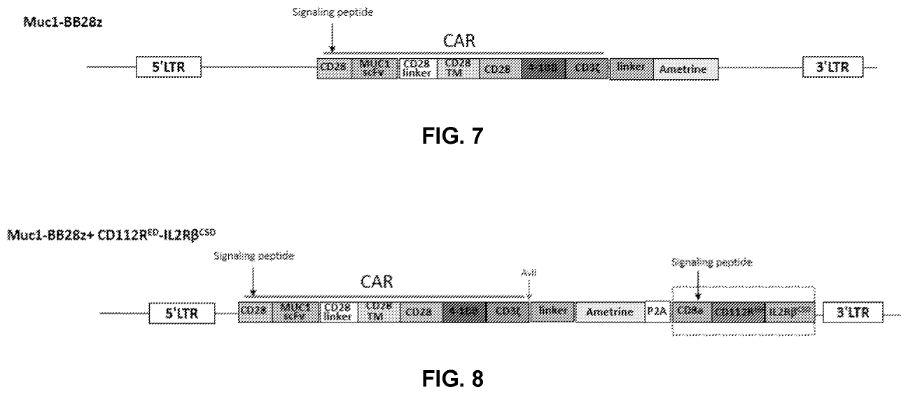

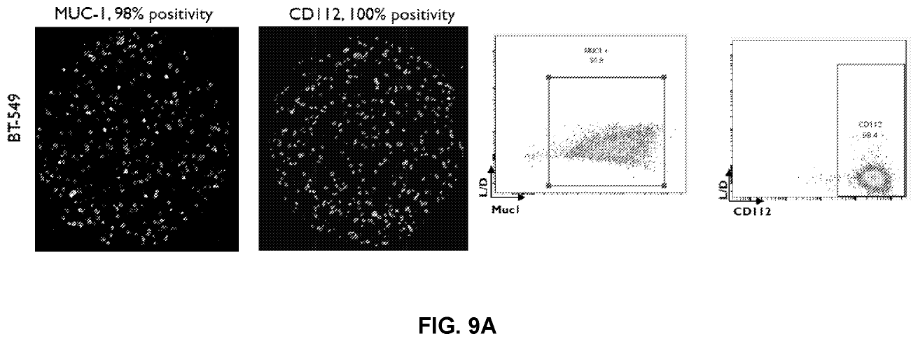

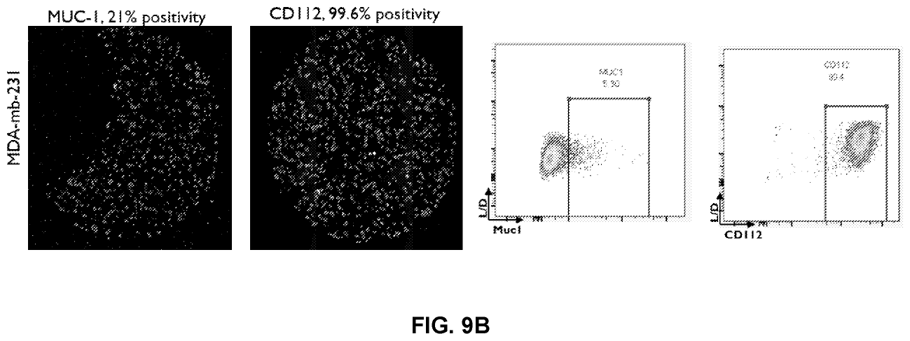

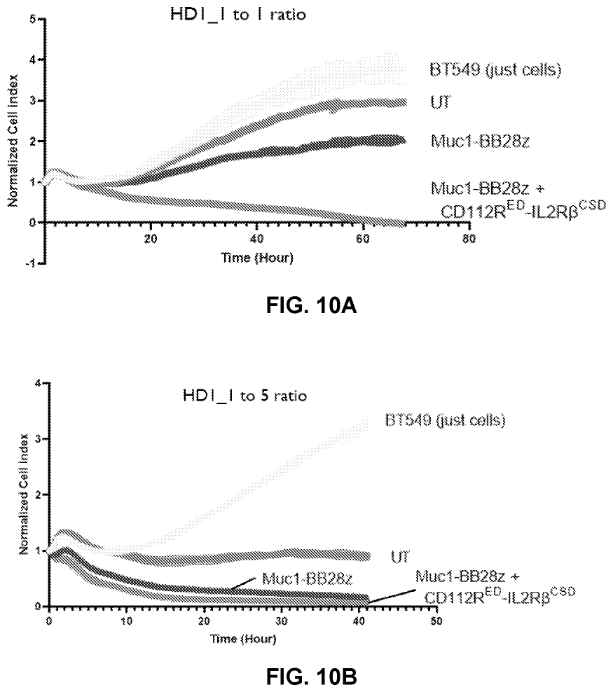

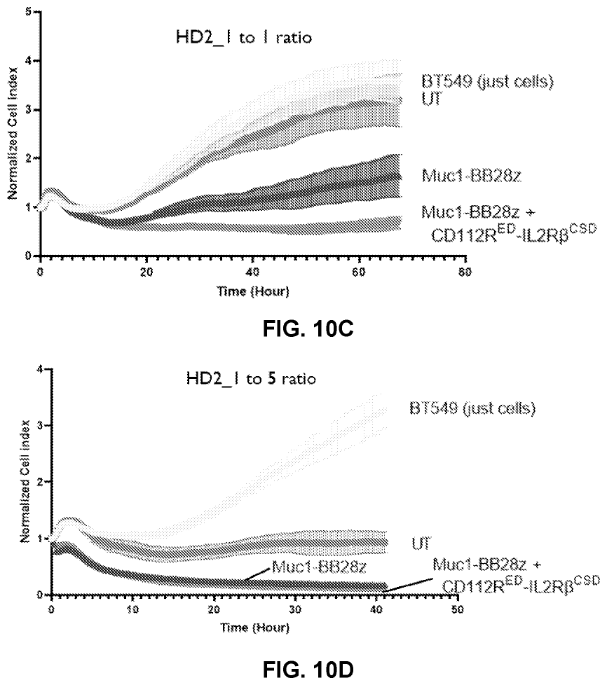

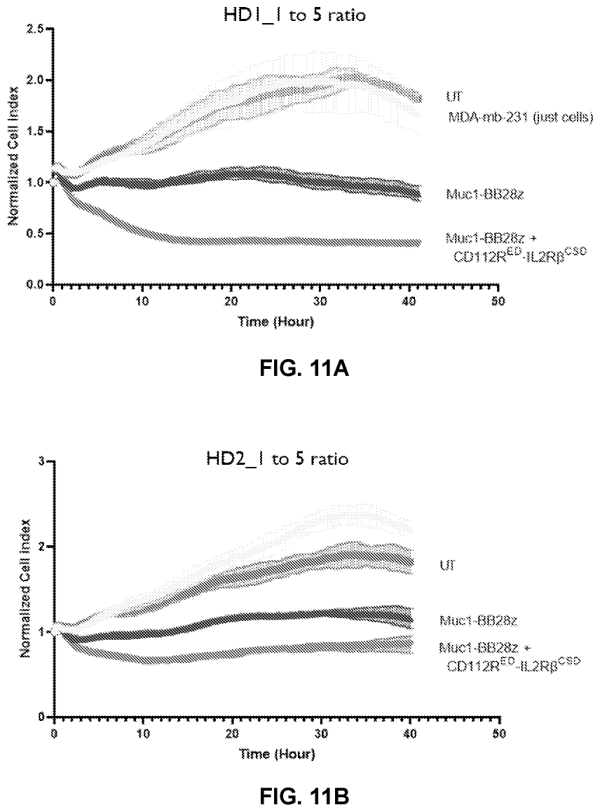

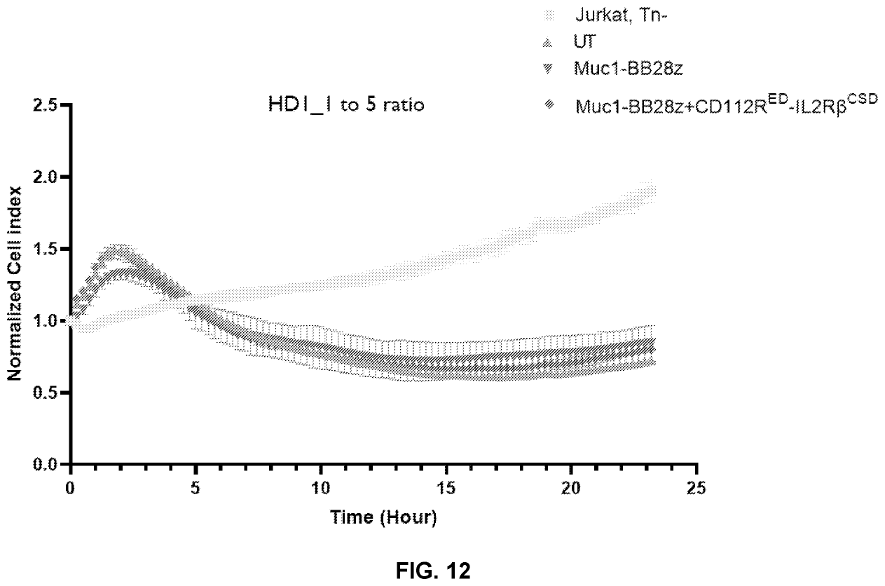

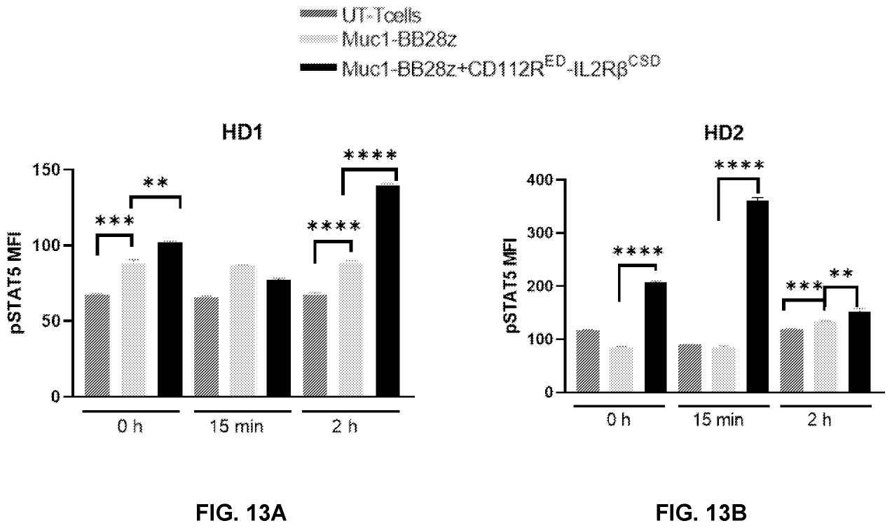

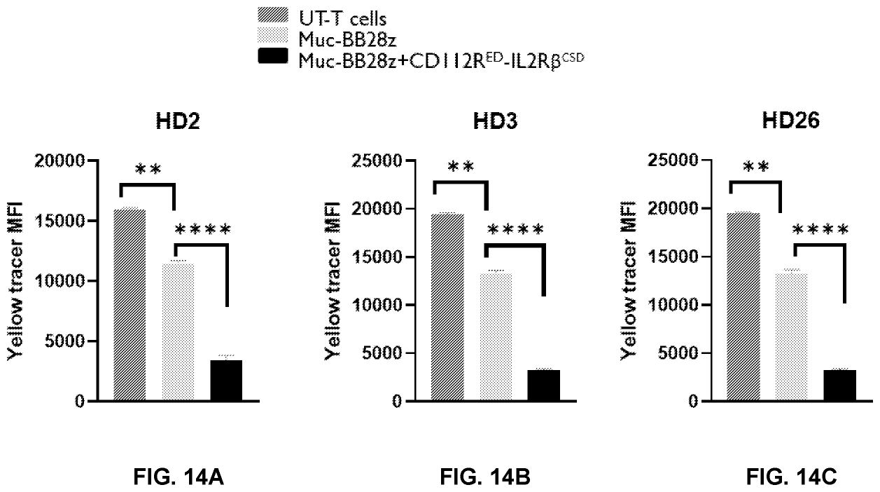

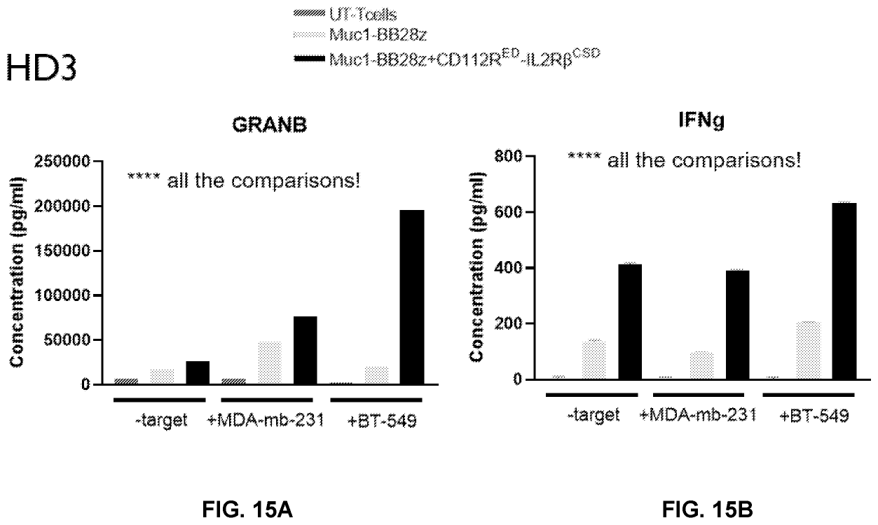

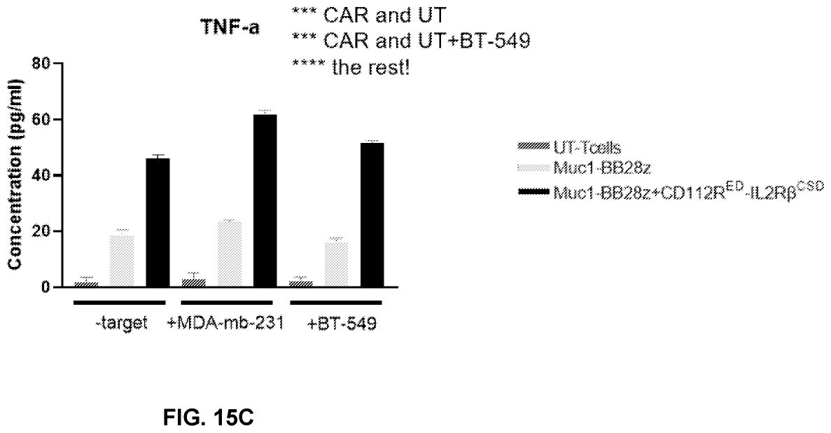

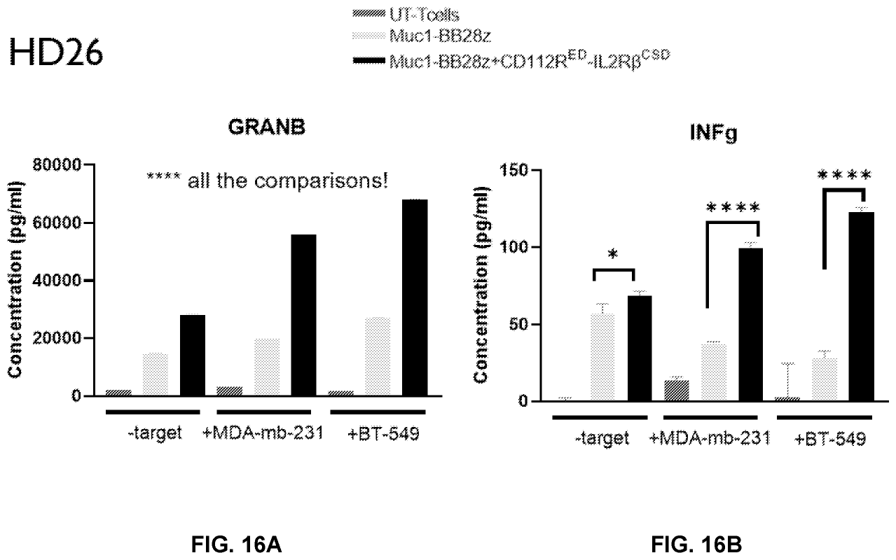

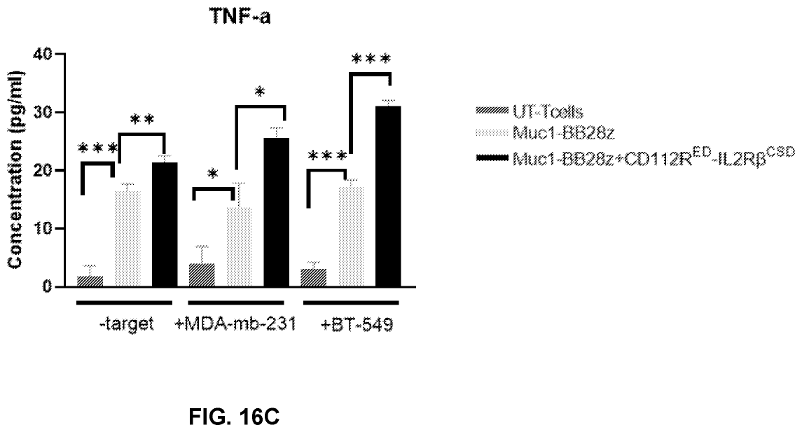

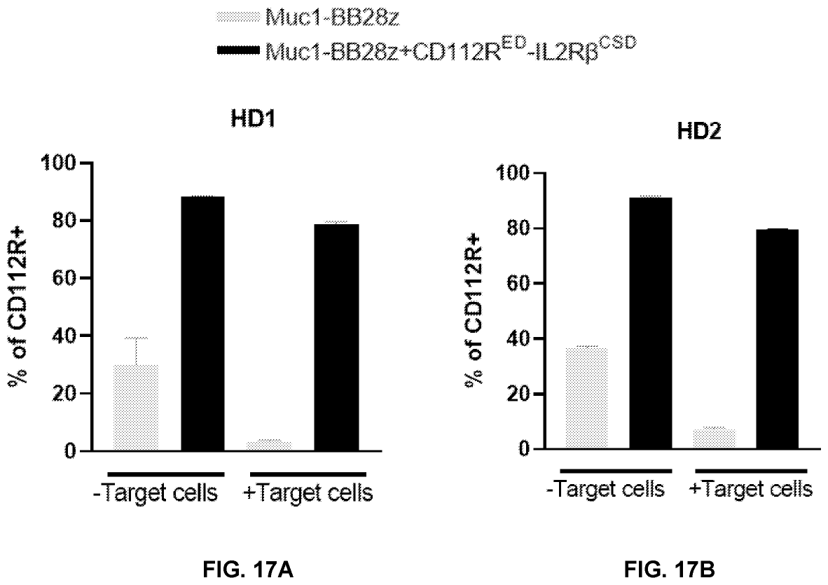

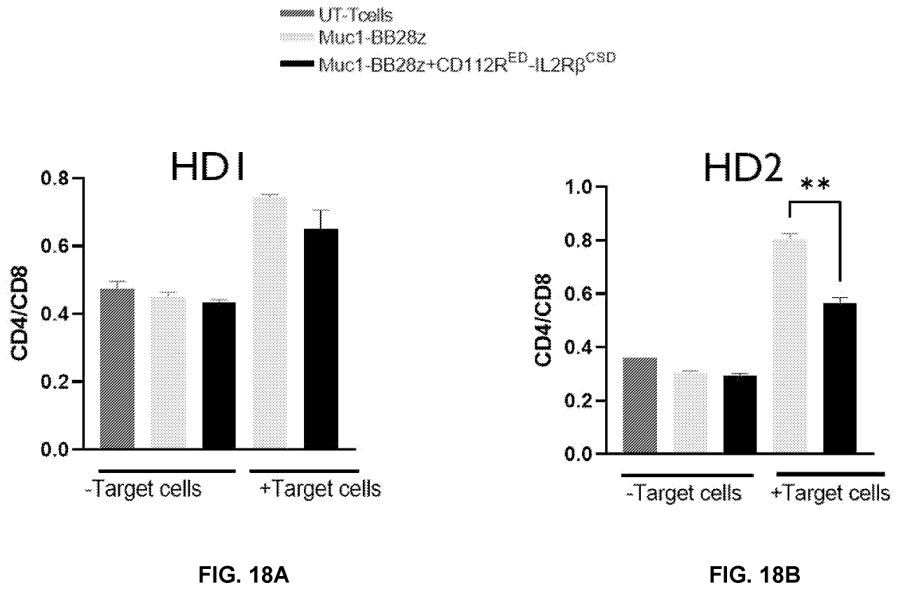

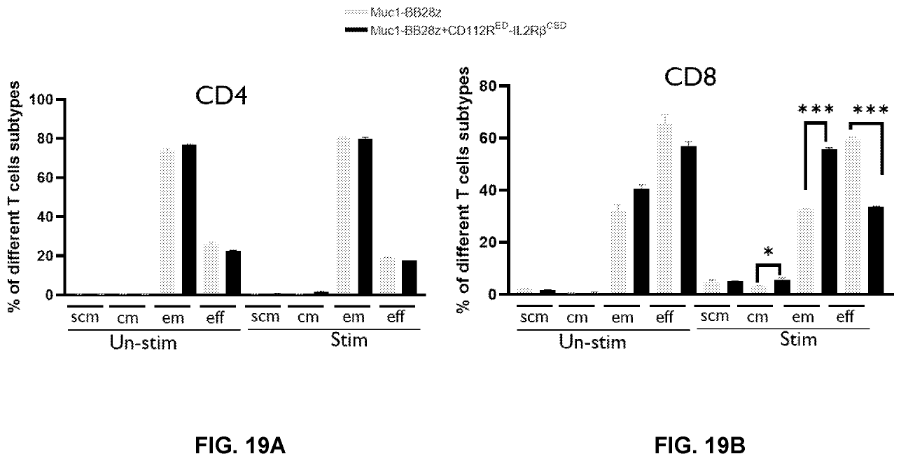

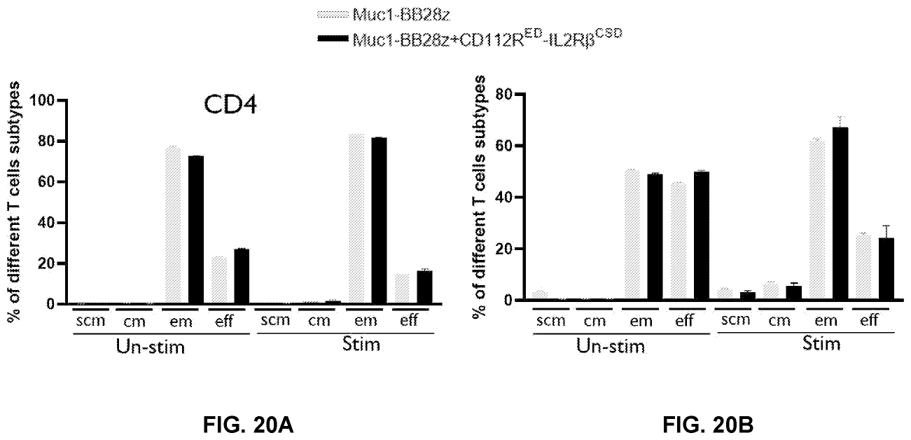

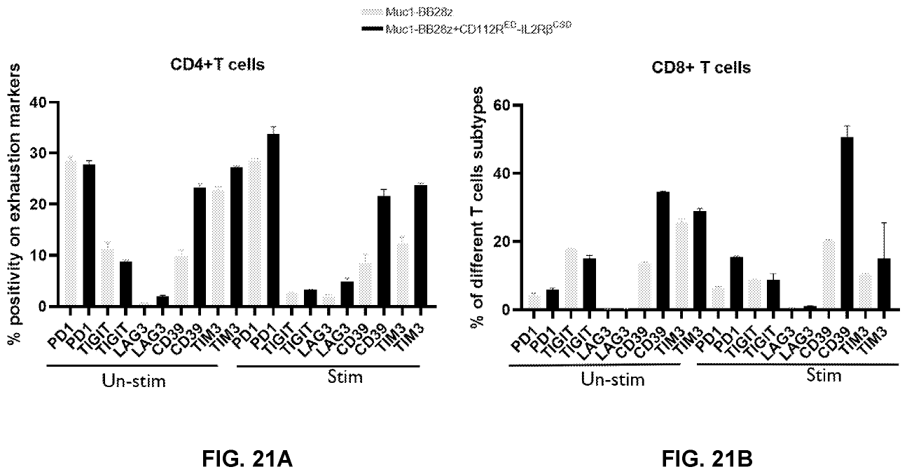

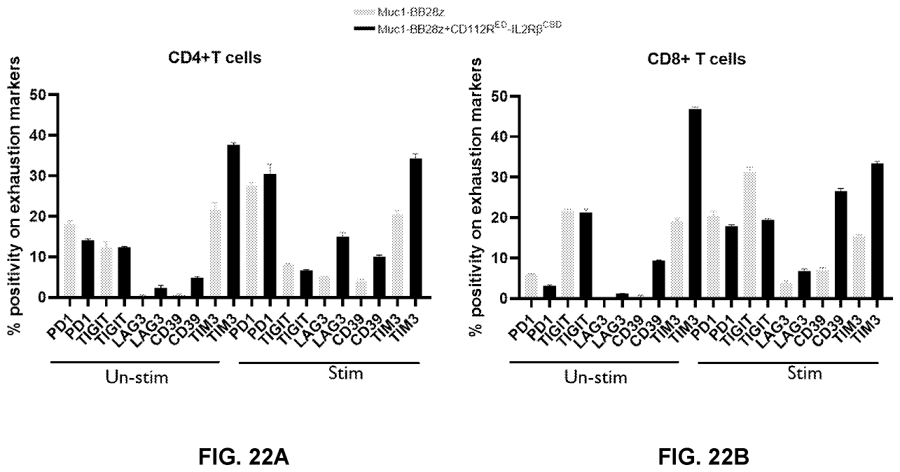

The targets and inhibitory ligands that were identified using RNA-seq ware validated on protein level using a TNBC TMA containing 59 TNBC (n=2-3 punches for each sample), 2 normal breast (n=3 punches per sample), normal spleen, muscle, kidneys, lung, liver, GI and heart (n=3 each) and TNBC cell lines (Hs578t, MDA-mb-231, BT-549, MDA-mb-157). Multiplex immune fluorescent (mIF) was used for the staining after optimizing the antibodies. The resulting multi-layer TIFF images were loaded onto HALO (Indica Labs) for segmentation and quantitative digital image analyses (FIG. 2A-J), representative mlF image of a melanoma TME). For each marker, a positivity threshold within the nucleus or cytoplasm was determined per marker based on published staining patterns and intensity for that specific antibody.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A chimeric receptor comprising an extracellular domain of TIM-3, LAG-3, DR5, or CD112R and an intracellular domain of a pro-inflammatory interleukin and/or a co-stimulatory domain.

2. The chimeric receptor of claim 1, wherein the pro-inflammatory interleukin is selected from the group consisting of IL7Rα, IL2Rβ, IL15, IL21R, and IL12Rβ2.

3. The chimeric receptor of claim 1, wherein the co-stimulatory domain is selected from the group consisting of CD27, ICOS, OX40.

4. The chimeric receptor of claim 1, comprising an amino acid sequence selected from the group consisting of SEQ ID NOs:1 to 32.

5. An immune effector cell engineered to express a chimeric antigen receptor (CAR) polypeptide and the chimeric receptor of claim 1.

6. The immune effector cell of claim 5, wherein the cell is selected from the group consisting of an αβT cell, γδT cell, a Natural Killer (NK) cells, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, a regulatory T cell, or any combination thereof.

7. The immune effector cell of claim 5, wherein the CAR polypeptide comprises an antigen binding domain, a transmembrane domain, an intracellular signaling domain (ISD), and a co-stimulatory signaling region (CSR).

8. The immune effector cell of claim 7, wherein the antigen binding domain is a single-chain variable fragment (scFv) of an antibody comprising a variable heavy (V_{H}) domain and a variable light (V_{L}) domain.

9. The immune effector cell of claim 5, wherein the CAR polypeptide selectively binds MUC-1.

10. The immune effector cell of claim 9, wherein the MUC-1 antigen binding domain is a single-chain variable fragment (scFv) of an antibody comprising a variable heavy (V_{H}) domain having CDR1, CDR2 and CDR3 sequences and a variable light (V_{L}) domain having CDR1, CDR2 and CDR3 sequences, and wherein the CDR1 sequence of the V_{H} domain comprises the amino acid sequence YTFTDHAIH (SEQ ID NO: 118); CDR2 sequence of the V_{H} domain comprises the amino acid sequence WIGHFSPGNTDIKY (SEQ ID NO:119); CDR3 sequence of the V_{H} domain comprises the amino acid sequence KTSTFFFD (SEQ ID NO:120); CDR1 sequence of the V_{L} comprises the amino acid sequence SQSLLNSGDQKNY (SEQ ID NO:121); CDR2 sequence of the V_{L} domain comprises the amino acid sequence KLLIFWASTR (SEQ ID NO:122); and CDR3 sequence of the V_{L} domain comprises the amino acid sequence CONDYSY (SEQ ID NO:123).

11. The immune effector cell of claim 10, wherein the V_{H} domain comprises the amino acid sequence SEQ ID NO: 124 and wherein the V_{L} domain comprises the amino acid sequence SEQ ID NO: 125.

12. An immune effector cell engineered to express a chimeric antigen receptor (CAR) polypeptide and engineered to silence expression of checkpoint inhibitor receptors selected from the group consisting of TIM-3, LAG-3, and CD112R.

13. The immune effector cell of claim 12, wherein expression of the checkpoint inhibitor receptors is silenced with a shRNA.

14. A method of providing an anti-cancer immunity in a subject, the method comprising administering to the subject an effective amount of the immune effector cell of claim 5, thereby providing an anti-cancer immunity in the subject.

15. The method of claim 14, further comprising administering to the subject a checkpoint inhibitor.

16. The method of claim 15, wherein the checkpoint inhibitor comprises an anti-PD-1 antibody, anti-PD-L1 antibody, anti-CTLA-4 antibody, or a combination thereof.

17. The method of claim 14, wherein the cancer comprises a triple negative breast cancer (TNBC).

18. The method of claim 14, wherein the cancer is an epithelial cancer.

19. The method of claim 18, wherein the cancer is a breast, gastric, colorectal, cholangiocarcinoma, ovarian, or non-small cell lung cancer.

20. The method of claim 14, further comprising administering to the subject a vascular endothelial growth factor-A (VEGF-A) or vascular endothelial growth factor receptor (VEGFR) antagonist antibody
